(19) United States
(12) Patent Application Publication
(10) Pub. No.: US 2009/0042897 A1
(43) Pub. Date: Feb. 12, 2009

(54) Chemical Compounds

(75) Inventors: Jonathan Bentley, Abingdon (GB); Matteo Biagetti, Verona (IT); Thorsten Genski, Verona (IT); Silvia Rosalia Kopf, Verona (IT); Colin Philip Leslie, Verona (IT); Sergio Melotto, Verona (IT)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: GLAXO GROUP LIMITED, Greenford (GB)

(21) Appl. No.: 12/022,327

(22) Filed: Jan. 30, 2008

(30) Foreign Application Priority Data
Feb. 1, 2007 (GB) 0701962.3
Oct. 24, 2007 (GB) 0720880.4
Jan. 8, 2008 (GB) 0800267.7

(51) Int. Cl. C07D 413/02; A61K 31/501; A61K 31/4439; A61K 31/423; A61P 3/00; A61P 25/00; A61P 25/24

(52) U.S. Cl. 514/252.06; 546/15; 514/278; 544/230; 548/216; 514/375

(57) ABSTRACT

The present invention relates to novel compounds or a pharmaceutically acceptable salt or solvate thereof, selected from a group consisting of:
(trans)-8-({[1-(2-fluorophenyl)-1H-pyrazol-3-yl]amino}methyl)-3-(2-fluoro-3-pyridinyl)-1-oxa-3-azaspiro[4.5]decan-2-one;
(trans)-8-({[1-(2-fluorophenyl)-1H-pyrazol-3-yl]amino}methyl)-3-(3-pyridazinyl)-1-oxa-3-azaspiro[4.5]decan-2-one;
(trans)-8-({[1-(2-fluorophenyl)-1H-pyrazol-3-yl]amino}methyl)-3-(1-methyl-1H-pyrazol-3-yl)-1-oxa-3-azaspiro[4.5]decan-2-one;
   processes for their preparation, intermediates used in these processes, pharmaceutical compositions containing them and their use in therapy, as NPY Y5 receptor antagonists and as agents for the treatment and/or prophylaxis of eating disorders such as a binge eating disorder.

This application claims the benefit of GB Application No. 0701962.3, filed Feb. 1, 2007; GB Application No. 0720880.4, filed Oct. 24, 2007; and GB Application No. 0800267.7, filed Jan. 8, 2008, each incorporated herein by reference in their entirety.

The present invention relates to novel compounds, processes for their preparation, intermediates used in these processes, pharmaceutical compositions containing them and their use in therapy, as NPY Y5 receptor antagonists and as agents for the treatment and/or prophylaxis of eating disorders such as a binge eating disorder.

Neuropeptide Y (hereinafter referred to as NPY), a peptide consisting of 36 amino acids, was first isolated from porcine brain by Tatemoto et al. in 1982 [Nature, 296: 659 (1982)]. NPY is widely distributed in central and peripheral nervous systems and plays various roles as one of the most abundant peptides in the nervous system. NPY acts as an orexigenic substance in the central nervous system and markedly promotes fat accumulation via the mediation of the secretion of various hormones or the action of the nervous system. It is known that the continuous intracerebroventricular administration of NPY induces obesity and insulin resistance based on these actions (International Journal of Obesity, vol. 19: 517 (1995); Endocrinology, vol. 133: 1753 (1993)). It is also known that NPY has central effects that are related to diseases such as depression, anxiety, schizophrenia, pain, dementia and the like (Drugs, vol. 52, 371 (1996). Furthermore, in the periphery, NPY coexists with norepinephrine in sympathetic nerve endings and is involved in the tonicity of the sympathetic nervous system. It is known that peripheral administration of NPY causes vasoconstriction and enhances the activities of other vasoconstrictive substances such as norepinephrine (British Journal of Pharmacology, vol. 95: 419 (1988)). It is also reported that NPY could participate in the development of cardiac hypertrophy as a result of the sympathetic stimulation (Proceeding National Academic Science USA, Vol. 97, 1595 (2000)).

Endogenous receptor proteins that bind NPY and related peptides as ligands have been identified and distinguished, and several such proteins have been cloned and expressed. Six different receptor subtypes [Y1, Y2, Y3, Y4(PP), Y5, Y6] are recognised today based upon binding profile, pharmacology and/or composition if identity is known.

The Y5 subtype was isolated, characterized and reported recently in U.S. Pat. No. 5,602,024 (WO 96/16542). The effects mediated by the NPY Y5 receptor include eating stimulation and accumulation of fat (Nature, vol. 382, 168 (1996)); American Journal of Physiology, vol. 277, R1428 (1999)). It is reported that the NPY Y5 receptor also mediates some CNS effects, such as seizure and epilepsy, or pain and morphine withdrawal symptoms (Natural Medicine, vol. 3, 761 (1997); Proceeding Academic Science USA, vol. 96, 13518 (1999); The Journal of Pharmacology and Experimental Therapeutics, vol. 284, 633 (1998)). In the periphery, the NPY Y5 receptor is reported to be involved in diuresis and the hypoglycemic effect caused by NPY (British Journal of Pharmacology, vol. 120, 1335 (1998); Endocrinology, vol. 139, 3018 (1998)). NPY is also reported to enhance cardiac hypertrophy as a result of sympathetic accentuation (Proceeding National Academic Science USA, Vol. 97, 1595 (2000)).

The effects of NPY occur by binding to the NPY receptors in the central or peripheral nervous system. Therefore, the action of NPY can be prevented by blocking the binding to NPY receptors. Substances that antagonize NPY binding to NPY receptors may be useful for the prophylaxis or treatment of various diseases related to NPY, such as cardiovascular disorders (for example hypertension, nephropathy, heart disease, vasospasm), central nervous system disorders (for example bulimia, binge eating, depression, anxiety, seizure, epilepsy, dementia, pain, alcoholism, drug withdrawal), metabolic diseases (for example obesity, diabetes, hormone abnormality), sexual and reproductive dysfunction, gastro-intestinal motility disorder, respiratory disorder, inflammation or glaucoma and the like (Trends in Pharmacological Sciences, 15: 153 (1994); Life Science, 55, 551 (1994); Drugs, vol. 52, 371 (1996); The Journal of Allergy and Immunology, vol. 101, S345 (1998); Nature, vol. 396, 366 (1998); The Journal of Pharmacology and Experimental Therapeutics, vol. 284, 633 (1998); Trends in Pharmacological Science, vol. 20, 104 (1999); Proceeding National Academic Science USA, vol. 97, 1595 (2000)).

The object of the present invention is to provide compounds of formula (I) or a pharmaceutically acceptable salt or solvate thereof:

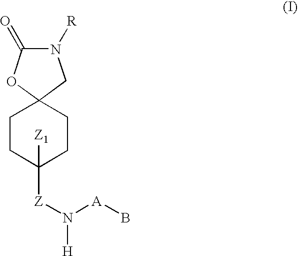

wherein
R is an aryl or heteroaryl; which may be substituted by one or more: halogen, C1-C4 alkyl, C1-C4 alkoxy, C1-C4 haloalkyl, C1-C4 haloalkoxy, cyano;
Z₁ is H, C1-C4 alkyl or F;
Z is CH₂, CH(C1-C4 alkyl), C(C1-C4 alkyl)₂ or a bond;
A is a 5 membered heteroaryl, which may be substituted by one or more: halogen, C1-C4 alkyl, C1-C4 alkoxy, C1-C4 haloalkyl, C1-C4 haloalkoxy, cyano;
B is hydrogen or is a 5-6 membered heteroaryl, or phenyl, which may be substituted by one or more: halogen, C1-C4 alkyl, C1-C4 alkoxy, C1-C4 haloalkyl, C1-C4 haloalkoxy, cyano; being A and B linked via any atom.

The compounds of the present invention may be in the form of and/or may be administered as a pharmaceutically acceptable salt. For a review on suitable salts see Berge et al, J. Pharm. Sci., 1977, 66, 1-19.

Typically, a pharmaceutically acceptable salt may be readily prepared by using a desired acid or base as appropriate. The salt may precipitate from solution and be collected by filtration or may be recovered by evaporation of the solvent.

Suitable pharmaceutically acceptable addition salts are formed from acids which form non-toxic salts and examples are hydrochloride, hydrobromide, hydroiodide, sulphate, bisulphate, nitrate, phosphate, hydrogen phosphate, acetate, maleate, malate, fumarate, lactate, tartrate, citrate, formate, gluconate, succinate, pyruvate, oxalate, oxaloacetate, trifluoroacetate, saccharate, benzoate, methanesulphonate, ethanesulphonate, benzenesulphonate, p-toluenesulphonate and isethionate.

Pharmaceutically acceptable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

Pharmaceutically acceptable salts may also be prepared from other salts, including other pharmaceutically acceptable salts, of the compound of formula (I) using conventional methods.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention.

In addition, prodrugs are also included within the context of this invention.

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews (1996) 19 (2) 115-130, each of which are incorporated herein by reference.

The term prodrug also encompasses any covalently bonded carriers that release a compound of structure (I) in vivo when such a prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein amine groups are bonded to any group that, when administered to a patient, cleaves to form the amine groups. Thus, representative examples of prodrugs include (but are not limited to) acetate, formate and benzoate derivatives of amine functional groups of the compounds of structure (I).

With regard to stereoisomers, the compounds of formula (I) may have one or more asymmetric carbon atoms and may occur as racemates, racemic mixtures and as individual enantiomers or diastereomers. All such isomeric forms are included within the present invention, including mixtures thereof.

When a specific enantiomer of a compound of formula (I) is required, this may be obtained for example by resolution of a corresponding enantiomeric mixture of a compound of formula (I) using conventional methods, such as H.P.L.C. of the corresponding racemate using a suitable chiral support or by fractional crystallisation of the diastereoisomeric salts formed by reaction of the corresponding racemate with a suitable optically active acid or base, as appropriate.

Or a specific enantiomer may also be prepared from a corresponding optically pure intermediate.

Separation of diastereoisomers or cis and trans isomers or syn and anti isomers may be achieved by conventional techniques, e.g. by fractional crystallisation, chromatography or H.P.L.C. of a stereoisomeric mixture.

Furthermore, some of the crystalline forms of the compounds of structure (I) may exist as polymorphs, which are included in the present invention.

The term C1-C4 alkyl as used herein as a group or a part of the group refers to a linear or branched alkyl group containing from 1 to 4 carbon atoms; examples of such groups include methyl, ethyl, propyl, isopropyl, n-butyl, isobutyl, tert butyl.

The term halogen refers to a fluorine, chlorine, bromine or iodine atom.

The term halo C1-C4 alkyl means an alkyl group having one to 4 carbon atoms and wherein at least one hydrogen atom is replaced with halogen such as for example a trifluoromethyl group and the like.

The term C1-C4 alkoxy group may be a linear or a branched chain alkoxy group, for example methoxy, ethoxy, propoxy, prop-2-oxy, butoxy, but-2-oxy or methylprop-2-oxy and the like.

The term halo C1-C4 alkoxy group may be a C1-C4 alkoxy group as defined before substituted with at least one halogen, preferably fluorine, such as OCHF₂, or OCF₃.

The term aryl means an aromatic carbocyclic moiety of 6 to 12 members. Representative aryl include (but are not limited to): phenyl, biphenyl or naphthyl.

The term heteroaryl means an aromatic heterocycle ring of 5 to 10 members and having at least one heteroatom selected from nitrogen, oxygen and sulfur, and containing at least 1 carbon atom, including both mono- and bicyclic ring systems.

Representative heteroaryls include (but are not limited to): furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, triazolyl, tetrazolyl, quinazolinyl, benzodioxolyl, benzothiadiazolyl, benzooxadiazolyl, imidazo[1,2-a]pyrazinyl, isothiazolyl, thiadiazolyl, [1,2,4]thiazol[1,5-9]pyridinyl.

Representative 5 membered heteroaryls include (but are not limited to): furyl, thiophenyl, pyrrolyl, oxazolyl, isooxazolyl, pyrazolyl, imidazolyl, thiazolyl, isothiazolyl, triazolyl, tetrazolyl, isothiazolyl, thiadiazolyl.

Representative 5-6 membered heteroaryls include (but are not limited to): furyl, thiophenyl, pyrrolyl, indolyl, pyridyl, oxazolyl, isooxazolyl, pyrazolyl, imidazolyl, thiazolyl, isothiazolyl, pyridazinyl, pyrimidinyl, pyrazinyl, triazinyl, triazolyl, tetrazolyl, isothiazolyl, thiadiazolyl.

With regard to stereoisomers, the compounds of formula (I),

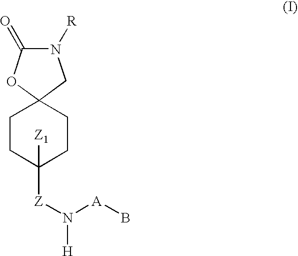

can exist as two stereoisomers represented by formulas (Ia) and (Ib).

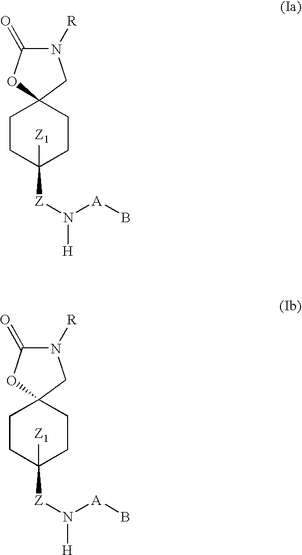

In one embodiment compound of formula (Ia) are provided in which the stereochemistry is “cis”, except when Z₁ is F wherein the stereochemistry is “trans”. In another embodiment of the present invention, compounds of formula (Ib) are provided and in which the stereochemistry is “trans”, except when Z₁ is F wherein the stereochemistry is “cis”.

“Trans” stereochemistry is due to highest priority groups, according to Kahn-Prelog-Ingold classification, attached to the cyclohexane ring being on opposite sides of the cyclohexane ring. “Trans” stereochemistry can be designated also as “trans configuration” or “anti”; in the case of formula (I) the description (5r,8r) can also be used to describe the “trans” stereochemistry.

In one aspect, the present invention provides compounds of formula (I), (Ia) and (Ib) in which:
R is phenyl or furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, triazolyl, tetrazolyl, quinazolinyl, benzodioxolyl, benzothiadiazolyl, benzooxadiazolyl, imidazo[1,2-a]pyrazinyl, isothiazolyl, thiadiazolyl, [1,2,4]thiazol[1,5-9]pyridinyl; which may be substituted by one or more: halogen, C1-C4 alkyl, C1-C4 alkoxy, C1-C4 haloalkyl, C1-C4 haloalkoxy, cyano;
A is selected from a group consisting of: furyl, thiophenyl, pyrrolyl, oxazolyl, isooxazolyl, pyrazolyl, imidazolyl, thiazolyl, isothiazolyl, triazolyl, tetrazolyl, isothiazolyl, thiadiazolyl; which may be substituted by one or more: halogen, C1-C4 alkyl, C1-C4 alkoxy, C1-C4 haloalkyl, C1-C4 haloalkoxy, cyano;
B is phenyl or pyridine, which may be substituted by one or more: halogen, C1-C4 alkyl, C1-C4 alkoxy, C1-C4 haloalkyl, C1-C4 haloalkoxy, cyano.

Example compounds of the present invention include:
(cis)3-phenyl-8-({[4-(2-pyridinyl)-1,3-thiazol-2-yl]amino}methyl)-1-oxa-3-azaspiro[4.5]decan-2-one;
(trans)-3-phenyl-8-({[4-(2-pyridinyl)-1,3-thiazol-2-yl]amino}methyl)-1-oxa-3-azaspiro-[4.5]decan-2-one;
(trans)-8-({[4-(6-methyl-2-pyridinyl)-1,3-thiazol-2-yl]amino}methyl)-3-phenyl-1-oxa-3-azaspiro[4.5]decan-2-one;
(trans)-8-({[4-(6-methyl-2-pyridinyl)-1,3-thiazol-2-yl]amino}methyl)-3-phenyl-1-oxa-3-azaspiro[4.5]decan-2-one;
(trans)-8-({[4-(3-methyl-2-pyridinyl)-1,3-thiazol-2-yl]amino}methyl)-3-phenyl-1-oxa-3-azaspiro[4.5]decan-2-one;
(trans)-8-({[4-(3-methyl-2-pyridinyl)-1,3-thiazol-2-yl]amino}methyl)-3-phenyl-1-oxa-3-azaspiro[4.5]decan-2-one;
(trans)-3-(2-pyridinyl)-8-({[4-(2-pyridinyl)-1,3-thiazol-2-yl]amino}methyl)-1-oxa-3-azaspiro[4.5]decan-2-one;
(trans)-3-(4-fluorophenyl)-8-({[4-(2-pyridinyl)-1,3-thiazol-2-yl]amino}methyl)-1-oxa-3-azaspiro[4.5]decan-2-one;
(trans)-3-(2-fluorophenyl)-8-({[4-(2-pyridinyl)-1,3-thiazol-2-yl]amino}methyl)-1-oxa-3-azaspiro[4.5]decan-2-one;
(cis)-3-(3-pyridinyl)-8-({[4-(2-pyridinyl)-1,3-thiazol-2-yl]amino}methyl)-1-oxa-3-azaspiro-[4.5]decan-2-one;
(trans)-3-(3-pyridinyl)-8-({[4-(2-pyridinyl)-1,3-thiazol-2-yl]amino}methyl)-1-oxa-3-azaspiro[4.5]decan-2-one;
(trans)-8-({[4-(3-fluoro-2-pyridinyl)-1,3-thiazol-2-yl]amino}methyl)-3-(3-pyridinyl)-1-oxa-3-azaspiro[4.5]decan-2-one;
(trans)-3-(2-methyl-3-pyridinyl)-8-({[4-(2-pyridinyl)-1,3-thiazol-2-yl]amino}methyl)-1-oxa-3-azaspiro[4.5]decan-2-one;
(trans)-8-({[4-(2-pyridinyl)-1,3-thiazol-2-yl]amino}methyl)-3-(5-pyrimidinyl)-1-oxa-3-azaspiro[4.5]decan-2-one;
(cis)-8-methyl-3-(2-pyridinyl)-8-({[4-(2-pyridinyl)-1,3-thiazol-2-yl]amino}methyl)-1-oxa-3-azaspiro[4.5]decan-2-one;
(trans)-8-methyl-3-(2-pyridinyl)-8-({[4-(2-pyridinyl)-1,3-thiazol-2-yl]amino}methyl)-1-oxa-3-azaspiro[4.5]decan-2-one;
(trans)-3-(6-methyl-2-pyridinyl)-8-({[4-(2-pyridinyl)-1,3-thiazol-2-yl]amino}methyl)-1-oxa-3-azaspiro[4.5]decan-2-one;
(trans)-3-(6-fluoro-2-pyridinyl)-8-({[4-(2-pyridinyl)-1,3-thiazol-2-yl]amino}methyl)-1-oxa-3-azaspiro[4.5]decan-2-one;
(trans)-3-(2-pyridinyl)-8-(1-{[4-(2-pyridinyl)-1,3-thiazol-2-yl]amino}ethyl)-1-oxa-3-azaspiro[4.5]decan-2-one;
(trans)-8-({[5-fluoro-4-(2-pyridinyl)-1,3-thiazol-2-yl]amino}methyl)-3-(2-pyridinyl)-1-oxa-3-azaspiro[4.5]decan-2-one;
(trans)-8-({[1-(2-fluorophenyl)-1H-pyrazol-3-yl]amino}methyl)-3-(2-fluoro-3-pyridinyl)-1-oxa-3-azaspiro[4.5]decan-2-one;
(trans)-8-({[1-(2-fluorophenyl)-1H-pyrazol-3-yl]amino}methyl)-3-(3-pyridazinyl)-1-oxa-3-azaspiro[4.5]decan-2-one;
(trans)-8-({[1-(2-fluorophenyl)-1H-pyrazol-3-yl]amino}methyl)-3-(1-methyl-1H-pyrazol-3-yl)-1-oxa-3-azaspiro[4.5]decan-2-one;
(trans)-8-({[1-(2-fluorophenyl)-1H-pyrazol-3-yl]amino}methyl)-3-[5-(trifluoromethyl)-3-pyridinyl]-1-oxa-3-azaspiro[4.5]decan-2-one;
(trans)-8-({[1-(2-fluorophenyl)-1H-pyrazol-3-yl]amino}methyl)-3-(2-pyrazinyl)-1-oxa-3-azaspiro[4.5]decan-2-one;
(trans)-3-(2,1,3-benzothiadiazol-5-yl)-8-({[1-(2-fluorophenyl)-1H-pyrazol-3-yl]amino}methyl)-1-oxa-3-azaspiro[4.5]decan-2-one;
(trans)-3-(1,3-benzodioxol-5-yl)-8-({[1-(2-fluorophenyl)-1H-pyrazol-3-yl]amino}methyl)-1-oxa-3-azaspiro[4.5]decan-2-one;
(trans)-8-({[1-(2-fluorophenyl)-1H-pyrazol-3-yl]amino}methyl)-3-[2-(methyloxy)-5-pyrimidinyl]-1-oxa-3-azaspiro[4.5]decan-2-one;
(trans)-8-({[1-(2-fluorophenyl)-1H-pyrazol-3-yl]amino}methyl)-3-(1-oxido-3-pyridinyl)-1-oxa-3-azaspiro[4.5]decan-2-one;
(trans)-8-({[1-(2-fluorophenyl)-1H-pyrazol-3-yl]amino}methyl)-3-(2-methyl-3-pyridinyl)-1-oxa-3-azaspiro[4.5]decan-2-one;
(trans)-8-({[1-(2-fluorophenyl)-1H-pyrazol-3-yl]amino}methyl)-3-(5-pyrimidinyl)-1-oxa-3-azaspiro[4.5]decan-2-one;
(trans)-8-({[1-(2-fluorophenyl)-1H-pyrazol-3-yl]amino}methyl)-3-(5-methyl-2-pyridinyl)-1-oxa-3-azaspiro[4.5]decan-2-one;
(trans)-8-({[1-(2-fluorophenyl)-1H-pyrazol-3-yl]amino}methyl)-3-(6-methyl-3-pyridinyl)-1-oxa-3-azaspiro[4.5]decan-2-one;
(trans)-8-({[1-(2-fluorophenyl)-1H-pyrazol-3-yl]amino}methyl)-3-(2-methyl-4-pyridinyl)-1-oxa-3-azaspiro[4.5]decan-2-one;
(trans)-8-({[1-(2-fluorophenyl)-1H-pyrazol-3-yl]amino}methyl)-3-[6-(methyloxy)-3-pyridinyl]-1-oxa-3-azaspiro[4.5]decan-2-one;
(trans)-8-({[1-(2-fluorophenyl)-1H-pyrazol-3-yl]amino}methyl)-3-(6-fluoro-3-pyridinyl)-1-oxa-3-azaspiro[4.5]decan-2-one;
(trans)-8-({[1-(2-fluorophenyl)-1H-pyrazol-3-yl]amino}methyl)-3-imidazo[1,2-a]pyridin-6-yl-1-oxa-3-azaspiro[4.5]decan-2-one;
(trans)-3-(3-fluoro-6-methyl-2-pyridinyl)-8-({[1-(2-fluorophenyl)-1H-pyrazol-3-yl]amino}methyl)-1-oxa-3-azaspiro[4.5]decan-2-one;
(trans)-8-({[1-(2-fluorophenyl)-1H-pyrazol-3-yl]amino}methyl)-3-(1,3-thiazol-2-yl)-1-oxa-3-azaspiro[4.5]decan-2-one;
4-[(trans)-8-({[1-(2-fluorophenyl)-1H-pyrazol-3-yl]amino}methyl)-2-oxo-1-oxa-3-azaspiro-[4.5]dec-3-yl]benzonitrile;
3-[(trans)-8-({[1-(2-fluorophenyl)-1H-pyrazol-3-yl]amino}methyl)-2-oxo-1-oxa-3-azaspiro-[4.5]dec-3-yl]benzonitrile;
3-[(trans)-8-({[1-(2-fluorophenyl)-1H-pyrazol-3-yl]amino}methyl)-2-oxo-1-oxa-3-azaspiro-[4.5]dec-3-yl]benzonitrile;
(trans)-8-({[1-(2-fluorophenyl)-1H-pyrazol-3-yl]amino}methyl)-3-(5-fluoro-3-pyridinyl)-1-oxa-3-azaspiro[4.5]decan-2-one;
(trans)-8-({[1-(2-fluorophenyl)-1H-pyrazol-3-yl]amino}methyl)-3-(1-methyl-1H-imidazol-5-yl)-1-oxa-3-azaspiro[4.5]decan-2-one;
(trans)-8-({[1-(2-fluorophenyl)-1H-pyrazol-3-yl]amino}methyl)-3-imidazo[1,2-a]pyrazin-3-yl-1-oxa-3-azaspiro[4.5]decan-2-one;
(trans)-8-({[1-(2-fluorophenyl)-1H-pyrazol-3-yl]amino}methyl)-3-(1-methyl-6-oxo-1,6-dihydro-3-pyridinyl)-1-oxa-3-azaspiro[4.5]decan-2-one;
(trans)-8-({[1-(2-fluorophenyl)-1H-pyrazol-3-yl]amino}methyl)-3-imidazo[1,2-a]pyridin-7-yl-1-oxa-3-azaspiro[4.5]decan-2-one;
(trans)-3-(2,1,3-benzoxadiazol-5-yl)-8-({[1-(2-fluorophenyl)-1H-pyrazol-3-yl]amino}methyl)-1-oxa-3-azaspiro[4.5]decan-2-one;
(trans)-8-({[1-(2-fluorophenyl)-1H-pyrazol-3-yl]amino}methyl)-3-(3-methyl-5-isothiazolyl)-1-oxa-3-azaspiro[4.5]decan-2-one;
(trans)-8-({[1-(2-fluorophenyl)-1H-pyrazol-3-yl]amino}methyl)-3-(1-methyl-1H-imidazol-2-yl)-1-oxa-3-azaspiro[4.5]decan-2-one;
(trans)-8-({[1-(2-fluorophenyl)-1H-pyrazol-3-yl]amino}methyl)-3-(2-pyrimidinyl)-1-oxa-3-azaspiro[4.5]decan-2-one;
(trans)-3-(2-fluoro-6-methyl-3-pyridinyl)-8-({[1-(2-fluorophenyl)-1H-pyrazol-3-yl]amino}-methyl)-1-oxa-3-azaspiro[4.5]decan-2-one;
(trans)-8-({[1-(2-fluorophenyl)-1H-pyrazol-3-yl]amino}methyl)-3-(2-methyl-5-pyrimidinyl)-1-oxa-3-azaspiro[4.5]decan-2-one;
(trans)-8-({[1-(2-fluorophenyl)-1H-pyrazol-3-yl]amino}methyl)-3-(2-methyl-1,3-thiazol-4-yl)-1-oxa-3-azaspiro[4.5]decan-2-one;
(trans)-8-({[1-(2-fluorophenyl)-1H-pyrazol-3-yl]amino}methyl)-3-[2-(trifluoromethyl)-5-pyrimidinyl]-1-oxa-3-azaspiro[4.5]decan-2-one;
(trans)-8-({[1-(2-fluorophenyl)-1H-pyrazol-3-yl]amino}methyl)-3-(2-fluoro-4-pyridinyl)-1-oxa-3-azaspiro[4.5]decan-2-one;
(trans)-3-(2,6-dimethyl-4-pyridinyl)-8-({[1-(2-fluorophenyl)-1H-pyrazol-3-yl]amino}methyl)-1-oxa-3-azaspiro[4.5]decan-2-one;
(trans)-8-({[1-(2-fluorophenyl)-1H-pyrazol-3-yl]amino}methyl)-3-(4-pyridazinyl)-1-oxa-3-azaspiro[4.5]decan-2-one;
(trans)-8-({[1-(2-fluorophenyl)-1H-pyrazol-3-yl]amino}methyl)-3-(5-methyl-1,3,4-thiadiazol-2-yl)-1-oxa-3-azaspiro[4.5]decan-2-one;
(trans)-8-({[1-(2-fluorophenyl)-1H-pyrazol-3-yl]amino}methyl)-3-(3-pyridinyl)-1-oxa-3-azaspiro[4.5]decan-2-one;
(trans)-8-({[1-(2-fluorophenyl)-1H-pyrazol-3-yl]amino}methyl)-3-(1H-pyrazol-3-yl)-1-oxa-3-azaspiro[4.5]decan-2-one;
(trans)-8-({[1-(2-fluorophenyl)-1H-pyrazol-3-yl]amino}methyl)-3-(1H-pyrazol-4-yl)-1-oxa-3-azaspiro[4.5]decan-2-one;
(trans)-8-({[1-(2-fluorophenyl)-1H-pyrazol-3-yl]amino}methyl)-3-(2-pyridinyl)-1-oxa-3-azaspiro[4.5]decan-2-one;
8-fluoro-8-({[1-(2-fluorophenyl)-1H-pyrazol-3-yl]amino}methyl)-3-(2-pyridinyl)-1-oxa-3-azaspiro[4.5]decan-2-one;
8-fluoro-8-({[1-(2-fluorophenyl)-1H-pyrazol-3-yl]amino}methyl)-3-(2-pyridinyl)-1-oxa-3-azaspiro[4.5]decan-2-one;
(trans)-8-({[1-(2-fluorophenyl)-1H-pyrazol-3-yl]amino}methyl)-3-[1,2,4]triazolo[1,5-a]pyridin-6-yl-1-oxa-3-azaspiro[4.5]decan-2-one;
(trans)-8-({[1-(2-fluorophenyl)-1H-pyrazol-3-yl]amino}methyl)-3-(1-methyl-1H-pyrazol-4-yl)-1-oxa-3-azaspiro[4.5]decan-2-one;
(trans)-8-{[(5-phenyl-1H-pyrazol-3-yl)amino]methyl}-3-(3-pyridinyl)-1-oxa-3-azaspiro-[4.5]decan-2-one;
(cis)-8-({[1-(2-fluorophenyl)-1H-pyrazol-3-yl]amino}methyl)-3-(3-pyridinyl)-1-oxa-3-azaspiro[4.5]decan-2-one;
1-(2-fluorophenyl)-3-({[(trans)-2-oxo-3-(2-pyridinyl)-1-oxa-3-azaspiro[4.5]dec-8-yl]methyl}-amino)-1H-pyrazole-4-carbonitrile;
(trans)-8-{[(3-phenyl-5-isoxazolyl)amino]methyl}-3-(2-pyridinyl)-1-oxa-3-azaspiro[4.5]decan-2-one;
(trans)-3-phenyl-8-{[(3-phenyl-5-isoxazolyl)amino]methyl}-1-oxa-3-azaspiro[4.5]decan-2-one;
   or pharmaceutically acceptable salts, solvates thereof.

In one embodiment the present invention provides the following compounds or pharmaceutically acceptable salts or solvates thereof:
(trans)-8-({[1-(2-fluorophenyl)-1H-pyrazol-3-yl]amino}methyl)-3-(2-fluoro-3-pyridinyl)-1-oxa-3-azaspiro[4.5]decan-2-one;
(trans)-8-({[1-(2-fluorophenyl)-1H-pyrazol-3-yl]amino}methyl)-3-(3-pyridazinyl)-1-oxa-3-azaspiro[4.5]decan-2-one;
(trans)-8-({[1-(2-fluorophenyl)-1H-pyrazol-3-yl]amino}methyl)-3-(1-methyl-1H-pyrazol-3-yl)-1-oxa-3-azaspiro[4.5]decan-2-one.

In another embodiment the present invention provides compounds of formula (IIA), or a pharmaceutically acceptable salt or solvate thereof:

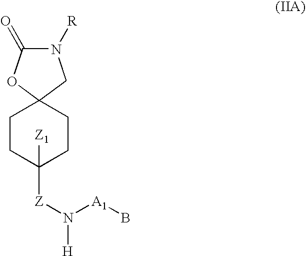

wherein
R is an aryl or heteroaryl; which may be substituted by one or more: halogen, C1-C4 alkyl, C1-C4 alkoxy, C1-C4 haloalkyl, C1-C4 haloalkoxy, cyano;
Z₁ is H, C1-C4 alkyl or F;
Z is CH₂, CH(C1-C4 alkyl), C(C1-C4 alkyl)₂ or a bond;
A₁ is thiazole, which may be substituted by one or more: halogen, C1-C4 alkyl, C1-C4 alkoxy, C1-C4 haloalkyl, C1-C4 haloalkoxy, cyano;
B is hydrogen or is a 5-6 membered heteroaryl, or phenyl, which may be substituted by one or more: halogen, C1-C4 alkyl, C1-C4 alkoxy, C1-C4 haloalkyl, C1-C4 haloalkoxy, cyano; being A and B linked via any atom.

In a further embodiment the present invention provides compounds of formula (IIB), or a pharmaceutically acceptable salt or solvate thereof:

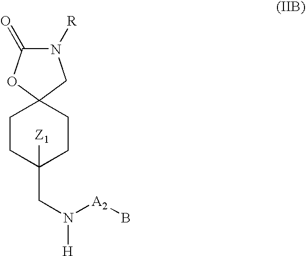

wherein
R is an aryl or heteroaryl; which may be substituted by one or more: halogen, C1-C4 alkyl, C1-C4 alkoxy, C1-C4 haloalkyl, C1-C4 haloalkoxy, cyano;
Z₁ is H, C1-C4 alkyl or F;
A₂ is pyrazole, which may be substituted by one or more: F, Cl, Br, C1-C4 alkyl, C1-C4 alkoxy, C1-C4 haloalkyl, C1-C4 haloalkoxy, cyano;
B is hydrogen or is a 5-6 membered heteroaryl, or phenyl, which may be substituted by one or more: halogen, C1-C4 alkyl, C1-C4 alkoxy, C1-C4 haloalkyl, C1-C4 haloalkoxy, cyano; being A and B linked via any atom.

In a further embodiment the present invention provides compounds of formula (IIC), or a pharmaceutically acceptable salt or solvate thereof:

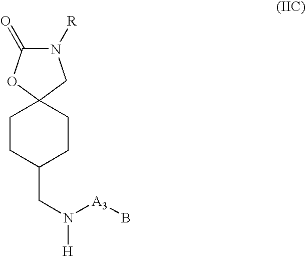

wherein
R is an aryl or heteroaryl; which may be substituted by one or more: halogen, C1-C4 alkyl, C1-C4 alkoxy, C1-C4 haloalkyl, C1-C4 haloalkoxy, cyano;
A₃ is isoxazole, which may be substituted by one or more: halogen, C1-C4 alkyl, C1-C4 alkoxy, C1-C4 haloalkyl, C1-C4 haloalkoxy, cyano;
B is hydrogen or is a 5-6 membered heteroaryl, or phenyl, which may be substituted by one or more: halogen, C1-C4 alkyl, C1-C4 alkoxy, C1-C4 haloalkyl, C1-C4 haloalkoxy, cyano; being A and B linked via any atom.

In general, the compounds of formula (I) may be made according to the organic synthesis techniques known to those skilled in this field, as well as by the representative methods set forth in the Examples.

Compounds of formula (I), and salts and solvates thereof, may be prepared by the general methods outlined hereinafter. In the following description, the groups R, Z, Z₁, A and B have the meanings as previously defined for compounds of formula (I) unless otherwise stated.

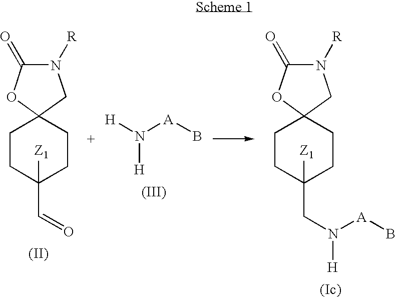

Compounds of formula (Ic), corresponding to compounds of formula (I) where Z=CH₂ can be prepared by reaction of aldehydes of formula (II) and amines of formula (III) in the presence of a reducing agent, for example sodium cyanoborohydride, sodium borohydride or sodium triacetoxyborohydride, optionally in the presence of a reagent, such as titanium tetraisopropoxide, titianium chloro-tri-isopropoxide and/or acetic acid, in a non-protic solvent such as dichloromethane. Compounds of formula (III) are commercially available e.g. 2-amino-4-(2-pyridyl)thiazole is available from, for example Fluorochem Ltd.; 2-amino-5-phenylpyrazine is available from Tokyo Chemical Industry Co., Ltd. Other amines can be prepared according to literature procedures or analogous procedures thereof e.g. 1-(2-fluorophenyl)-1H-pyrazole-3-amine is described in Journal of Organic Chemistry, 2005, 70(23), 9222-9229.

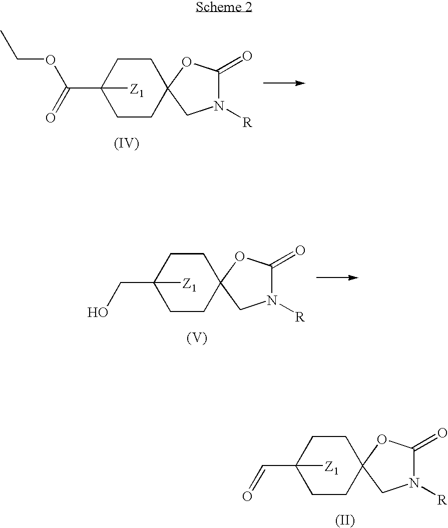

Aldehydes of formula (II) can be prepared by oxidation of alcohols of formula (V) using a reagent such as Dess-Martin periodinane, resin-supported IBX amide, DMPX, TPAP or ‘Swern’ oxidation conditions (oxalyl chloride/dimethyl sulfoxide in the presence of an amine base e.g. triethylamine or Hunig's base). Alcohols of formula (V) can be prepared from esters of formula (IV) via reduction with a reagent such as lithium aluminium hydride at a temperature below 0° C. in an aprotic solvent such as THF.

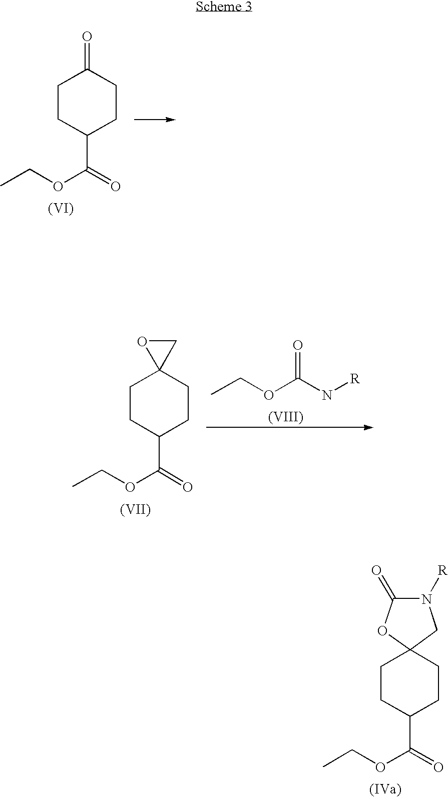

Esters of formula (IVa) can be prepared from an epoxide of formula (VII) and a carbamate of formula (VIII) in a solvent such as HPMA, DMPU or NMP in the presence of a base such as sodium tertiary-butoxide, sodium hydride or BEMP, preferably at a temperature greater than 100° C. An epoxide of formula (VII) can be prepared from a ketone (VI), which is commercially available from e.g. Sigma-Aldrich Chemicals, by treatment with trimethylsulphoxonium iodide or thrimethylsulphonium iodide in an aprotic solvent such as DMSO or acetonitrile in the presence of a base such as sodium hydride, potassium tertiary-butoxide or 2,8,9-triisobutyl-2,5,8,9-tetraaza-1-phosphabicyclo[3.3.3]undecane. Carbamates of formula (VIII) are commercially available from e.g. Sigma-Aldrich Chemicals.

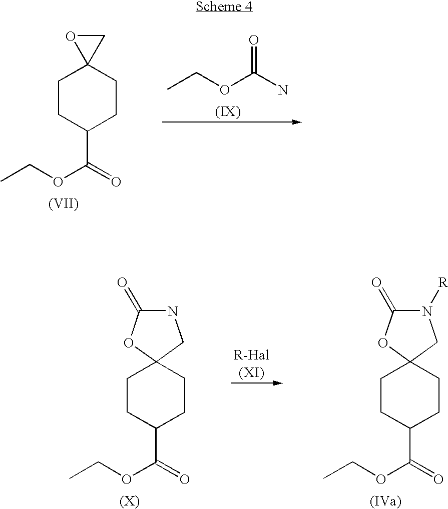

Esters of formula (IVa) can be prepared from esters of formula (X) and an aryl or heteroaryl halide of formula (XI). Suitable reactions conditions have been described in ‘Metal-Catalyzed Cross-Coupling Reactions (2nd Edition)’, 2004, 2, 699-760; Angewandte Chemie, International Edition, 2003, 42(44), 5400-5449 and the references therein. Aryl or heteroaryl halides of formula (XI) are commercially available from e.g. Sigma-Aldrich Chemicals. Esters of formula (X) can be prepared from an epoxide of formula (VII) and a carbamate of formula (IX) in a solvent such as HPMA, DMPU or NMP in the presence of a base such as potassium tertiary-butoxide, sodium hydride or BEMP, preferably at a temperature greater than 100° C. A carbamate of formula (IX) is commercially available from e.g. Sigma-Aldrich Chemicals.

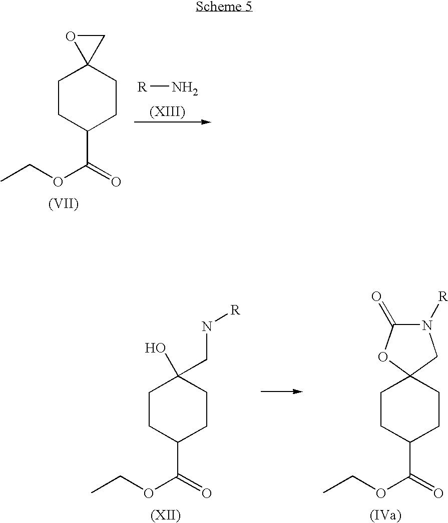

Alternatively, esters of formula (IVa) can be prepared from amino-alcohols of formula (XII) and a reagent such as phosgene, triphosgene, carbonyl di-imidazole, disuccinimidyl carbonate, carbon dioxide, an alkylchloroformate e.g. benzyl chloroformate or ethyl chloroformate, an aryl chloroformate e.g. phenyl chloroformate or a dialkyl pyrocarbonate e.g. di-tertiary-butyl di-carbonate (Boc anhydride), optionally in the presence of a base such as triethylamine in a solvent such as dichloromethane. Amino-alcohols of formula (XII) can be prepared from an epoxide of formula (VII) and amines of formula (XIII) in a protic solvent such as tertiary-butanol or ethoxyethanol at temperatures greater than 100° C. Amines of formula (XIII), such as aniline, are commercially available from e.g. Sigma-Aldrich Chemicals.

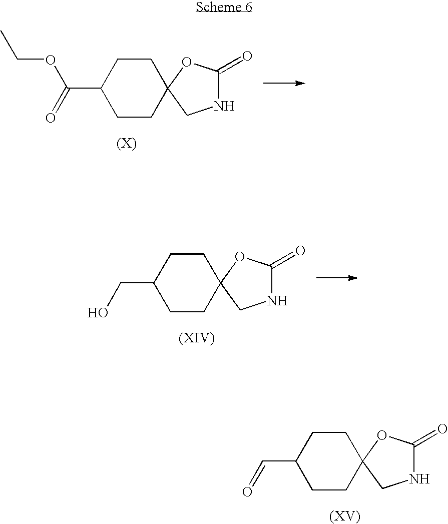

Aldehydes of formula (XV) can be prepared by oxidation of alcohols of formula (XIV) using a reagent such as Dess-Martin periodinane, resin-supported IBX amide, DMPX, TPAP or ‘Swern’ oxidation conditions (oxalyl chloride/dimethyl sulfoxide in the presence of an amine base e.g. triethylamine or Hunig's base). Alcohols of formula (XIV) can be prepared from esters of formula (X) via reduction with a reagent such as lithium aluminium hydride at a temperature below 0° C. in an aprotic solvent such as THF.

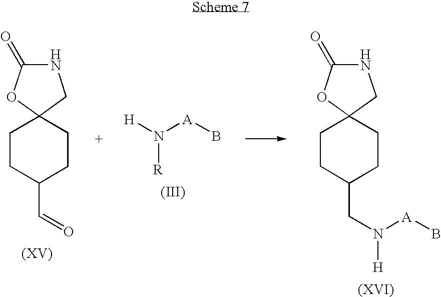

Compounds of formula (XVI can be prepared by reaction of aldehydes of formula (XV) and amines of formula (III) in the presence of a reducing agent, for example sodium cyanoborohydride, sodium borohydride or sodium triacetoxyborohydride, optionally in the presence of a reagent, such as titanium tetraisopropoxide, titianium chloro-tri-isopropoxide and/or acetic acid, in a non-protic solvent such as dichloromethane. Compounds of formula (III) are commercially available e.g. 2-amino-4-(2-pyridyl)thiazole is available from, for example Fluorochem Ltd.; 2-amino-5-phenylpyrazine is available from Tokyo Chemical Industry Co., Ltd. Other amines can be prepared according to literature procedures or analogous procedures thereof e.g. 1-(2-fluorophenyl)-1H-pyrazole-3-amine is described in Journal of Organic Chemistry, 2005, 70(23), 9222-9229.

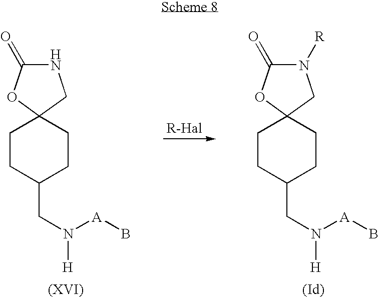

Compounds of formula (Id), corresponding to compounds (Ic) where Z₁=H, can be prepared from compounds of formula (XVI) and an aryl halide of formula (XI). Suitable reaction conditions have been described in ‘Metal-Catalyzed Cross-Coupling Reactions (2nd Edition)’, 2004, 2, 699-760; Angewandte Chemie, International Edition, 2003, 42(44), 5400-5449 and the references therein. Aryl halides of formula (XI) are commercially available from e.g. Sigma-Aldrich Chemicals.

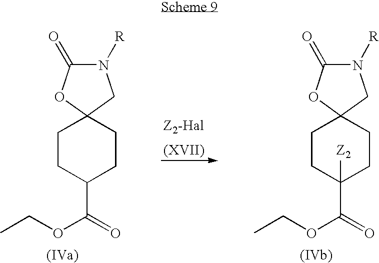

Esters of formula (IVb) can be prepared from esters of formula (IVa) by treatment with a base such as lithium diisopropylamide or sodium hexamethyldisilazide in an aprotic solvent such as THF, followed by treatment with an alkyl halide of formula (XVII) in which Z₂=C1-C4 alkyl. Alkyl halides of formula (XVII) are commercially available from e.g. Sigma-Aldrich Chemicals.

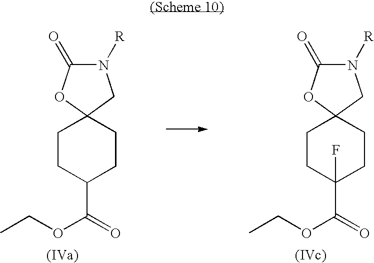

Esters of formula (IVc) can be prepared from esters of formula (IVa), by treatment with a base such as lithium diisopropylamide or sodium hexamethyldisilazide in an aprotic solvent such as THF, followed by treatment with an electrophilic fluorinating agent such as Selectfluor or N-fluorobenzenesulfonimide. Electrophilic fluorinating agents are commercially available from e.g. Sigma-Aldrich Chemicals.

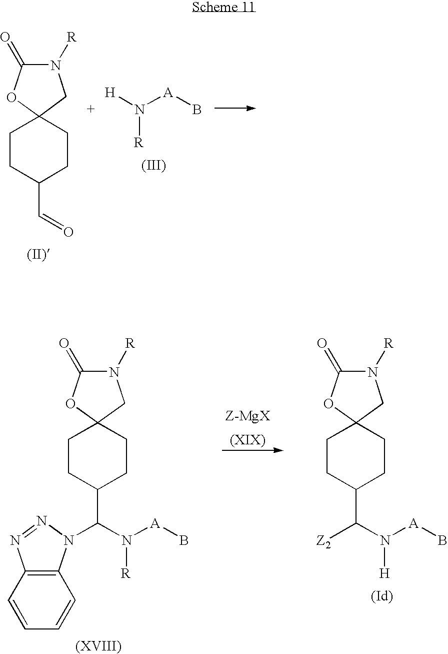

Compounds of formula (Id), corresponding to compounds of formula (I) where Z=CH(C1-C4 alkyl) and Z₁=H, can be prepared by reaction of a compound of formula (XVIII) with a Grignard reagent of formula (XIX) where Z₂=C1-C4 alkyl, in an aprotic solvent such as THF. Compounds of formula (XVIII) can be prepared by mixing aldehydes of formula (II)′, corresponding to compounds (II) where Z₁=H, and amines of formula (III) in the presence of benzotriazole in an aprotic solvent such a toluene preferably at temperatures greater than room temperature. Amines of formula (III) are commercially available e.g. 2-amino-4-(2-pyridyl)thiazole is available from, for example Fluorochem Ltd.; 2-amino-5-phenylpyrazine is available from Tokyo Chemical Industry Co., Ltd. Other amines can be prepared according to literature procedures or analogous procedures thereof e.g. 1-(2-fluorophenyl)-1H-pyrazole-3-amine is described in Journal of Organic Chemistry, 2005, 70(23), 9222-9229.

Those skilled in the art will appreciate that in the preparation of the compounds of the invention it may be necessary and/or desirable to protect one or more sensitive groups in the molecule to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl type protecting groups (e.g. benzyl, trityl, chlorotrityl).

The subject invention also includes isotopically-labelled compounds, which are identical to those recited in formula (I) and following, but for the fact that one or more atoms are replaced by an atom having an atomic mass or mass number different from the atomic mass or mass number usually found in nature. Examples of isotopes that can be incorporated into compounds of the invention and pharmaceutically acceptable salts thereof include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷O, ¹⁸O, ³¹P, ³²P, ³⁵S, ¹⁸F, ³⁶Cl, ¹²³I and ¹²⁵I.

Compounds of the present invention that contain the aforementioned isotopes and/or other isotopes of other atoms are within the scope of the present invention. Isotopically-labelled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Isotopically labelled compounds of formula I and following of this invention can generally be prepared by carrying out the procedures disclosed in the Schemes and/or in the Examples below, by substituting a readily available isotopically labelled reagent for a non-isotopically labelled reagent.

Compounds of the present invention are antagonists of the NPY Y5 receptor and as such are useful for the prevention and treatment of disorders or diseases associated with the NPY Y5 receptor sub-type, preferably for the treatment of eating disorders such as obesity, anorexia nervosa and bulimia nervosa, and other abnormal conditions, such as diabetes, hypertension, hyperlipemia, hypercholesterolemia, congestive heart failure, renal dysfunction, sexual/reproductive disorders, depression, anxiety, shock, epileptic seizure, memory loss, sleep disturbance, pain, migraine, cerebral hemorrhage, nasal congestion, gastrointestinal disorders, arthritis and immunodeficiency syndrome.

The compounds of the present invention may also be used in combination with other anti-obesity agents for increased efficacy in the prevention and treatment of eating disorders. Such agents would include, but not be limited to: sibutramine; dexfenfluramine; leptin; growth hormone secretagogue antagonists such as those disclosed and specifically described in U.S. Pat. No. 5,536,716; melanocortin agonists such as elanotan II; Beta-3 agonists such as those disclosed and specifically described in patent publications WO94/18161, WO95/29159, WO97/46556, WO98/04526 and WO98/32753; 5HT-2 agonists; orexin antagonists; melanin concentrating hormone antagonists; galanin antagonists; CCK agonists; GLP-1 agonists; corticotrophin releasing hormone agonists; Y1 antagonists, and CB1 antagonists

More particularly, compounds of the present invention are useful as agents for the treatment and/or prophylaxis of eating disorders such as a binge eating disorder.

The method of treatment of this invention comprises a method of antagonizing the NPY Y5 receptor and treating NPY Y5 receptor mediated diseases by administering to a patient in need of such treatment a non-toxic therapeutically effective amount of a compound of this invention that selectively antagonizes the NPY Y5 receptor in preference to the other NPY receptors.

Within the context of the present invention, the terms describing some indications used herein are classified in the Diagnostic and Statistical Manual of Mental Disorders, 4th Edition, published by the American Psychiatric Association (DSM-IV) and/or the International Classification of Diseases, 10th Edition (ICD-10). The various subtypes of the disorders mentioned herein are contemplated as part of the present invention. Numbers in brackets after the listed diseases below refer to the classification code in DSM-IV.

Depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90):

Anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00):

Substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide:

Sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type:

Eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; Binge Eating Disorder; and Eating Disorder Not Otherwise Specified (307.50):

Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9);

In a further embodiment the invention provides the use of a compound of formula (I), or a pharmaceutically acceptable salt or solvate thereof, in the preparation of a medicament for the treatment of a binge eating disorder.

In a further embodiment the present invention provides a method of treatment of a mammal suffering from a binge eating disorder, which comprises administering to said subject an effective amount of a compound of formula (I) or a pharmaceutically acceptable salt or solvate thereof.

In a further embodiment the invention provides the use of a compound of formula (I), or a pharmaceutically acceptable salt or solvate thereof, in the preparation of a medicament for the treatment of obesity.

In a further embodiment the present invention provides a method of treatment of a mammal suffering from obesity, which comprises administering to said subject an effective amount of a compound of formula (I) or a pharmaceutically acceptable salt or solvate thereof.

Compounds of formula (I) can be administered orally or parenterally and may be formulated in the form suitable for administration to provide an agent for treatment of various diseases related to NPY, which include, for example, cardiovascular disorders (for example hypertension, nephropathy, heart disease, vasospasm, arteriosclerosis), central nervous system disorders (for example bulimia, depression, anxiety, seizure, epilepsy, dementia, pain, alcoholism, drug withdrawal), metabolic diseases (for example obesity, diabetes, hormone abnormality, hypercholesterolemia, hyperlipidemia), sexual and reproductive dysfunction, gastro-intestinal motility disorder, respiratory disorder, inflammation or glaucoma and the like, preferably, bulimia, obesity, diabetes and the like.

While it is possible that, for use in therapy a therapeutically effective amount of a compound of formula (I), or a pharmaceutically acceptable salt or solvate thereof, may be administered as the raw chemical, it is preferable to present the active ingredient as a pharmaceutical composition. Thus, in a further embodiment the invention provides a pharmaceutical composition comprising a compound of formula (I), or a pharmaceutically acceptable salt or solvate thereof, in admixture with one or more pharmaceutically acceptable carriers, diluents, or excipients. The carrier(s), diluent(s) or excipient(s) must be acceptable in the sense of being compatible with the other ingredients of the formulation and not deleterious to the recipient thereof. In a further embodiment the invention also provides a process for the preparation of a pharmaceutical composition including admixing a compound of formula (I), or a pharmaceutically acceptable salt or solvate thereof, with one or more pharmaceutically acceptable carriers, diluents or excipients.

Pharmaceutical compositions of the invention may be formulated for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Therefore, the pharmaceutical compositions of the invention may be formulated, for example, as tablets, capsules, powders, granules, lozenges, creams or liquid preparations, such as oral or sterile parenteral solutions or suspensions. Such pharmaceutical formulations may be prepared by any method known in the art of pharmacy, for example by bringing into association the active ingredient with the carrier(s) or excipient(s).

Tablets and capsules for oral administration may be in unit dose presentation form, and may contain conventional excipients such as binding agents, for example syrup, acacia, gelatine, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatine, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams. The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Pharmaceutical formulations adapted for rectal administration may be presented as suppositories or as enemas.

Pharmaceutical formulations adapted for nasal administration wherein the carrier is a solid may include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

It should be understood that in addition to the ingredients particularly mentioned above, the formulations may include other agents conventional in the art having regard to the type of formulation in question.

The compounds of the present invention can be used in combination with other agents useful for treating metabolic and/or eating disorders. The individual components of such combinations can be administered separately at different times during the course of therapy or concurrently in divided or single combination forms. The instant invention is therefore to be understood as embracing all such regimes of simultaneous or alternating treatment and the term “administering” is to be interpreted accordingly. It will be understood that the scope of combinations of the compounds of this invention with other agents useful for treating metabolic and/or eating disorders includes in principle any combination with any pharmaceutical composition useful for treating metabolic and/or eating disorders.

A therapeutically effective amount of a compound of formula (I), or a pharmaceutically acceptable salt or solvate thereof will depend upon a number of factors including, for example, the age and weight of the human or other mammals, the precise condition requiring treatment and its severity, the nature of the formulation, and the route of administration, and will ultimately be at the discretion of the attendant physician or veterinarian. However, an effective amount of a compound of formula (I) for the treatment of disorders mediated by the NPY Y5 receptor will generally be in the range of 0.1 to 100 mg/kg body weight of recipient (mammal) per day and more usually in the range of 1 to 10 mg/kg body weight per day. Thus, for a 70 kg human adult, the actual amount per day would usually be from 70 to 700 mg and this amount may be given in a single dose per day or more usually in a number (such as two, three, four, five or six) of sub-doses per day such that the total daily dose is the same. An effective amount of a pharmaceutically acceptable salt or solvate thereof, may be determined as a proportion of the effective amount of the compound of formula (I) per se.

A compound of formula (I), or a pharmaceutically acceptable salt or solvate thereof for use in the instant invention may be used in combination with one or more other therapeutic agents. The invention thus provides in a further embodiment a combination comprising a compound of formula (I), or a pharmaceutically acceptable salt or solvate thereof together with a further therapeutic agent, which may be for example an additional anti-obesity agent. In a yet further embodiment the invention also provides the use of a combination comprising a compound of formula (I), or a pharmaceutically acceptable salt or solvate thereof with a further therapeutic agent in the treatment of disorders mediated by the NPY Y5 receptor.

When a compound of formula (I), or a pharmaceutically acceptable salt or solvate thereof is used in combination with one or more other therapeutic agents, the compounds may be administered either sequentially or simultaneously by any convenient route.

The combinations referred to above may conveniently be presented for use in the form of a pharmaceutical formulation and thus pharmaceutical formulations comprising a combination as defined above together with a pharmaceutically acceptable carrier or excipient comprise a further embodiment of the invention. The individual components of such combinations may be administered either sequentially or simultaneously in separate or combined pharmaceutical formulations.

When combined in the same formulation it will be appreciated that the two compounds must be stable and compatible with each other and the other components of the formulation and may be formulated for administration. When formulated separately they may be provided in any convenient formulation, conveniently in such a manner as are known for such compounds in the art.

When a compound is used in combination with a second therapeutic agent active against the same disease, the dose of each compound may differ from that when the compound is used alone. Appropriate doses will be readily appreciated by those skilled in the art.

The following Examples describe the laboratory synthesis of specific compounds of the invention and are not meant to limit the scope of the invention in any way with respect to compounds or processes. It is understood that, although specific reagents, solvents, temperatures and time periods are used, there are many possible equivalent alternatives that can be used to produce similar results. This invention is meant to include such equivalents.

### Experimental

The invention is illustrated by the Compounds described below.

### Abbreviations

DMAP 4-dimethylaminopyridine
DIPEA N,N-diisopropylethylamine
TEA triethylamine
TFA trifluoroacetic acid
EtOAc ethyl acetate
EDC.HCl N-(3-dimethylaminopropyl)-N′-ethylcarbodiimide hydrochloride
HOBt.H₂O 1-hydroxybenzyltriazole hydrate
DMSO dimethylsulfoxide
DCM dichloromethane
DMF N,N-dimethylformamide
HATU (O-7-azabenzotriazol-1-yl)-N,N,N′,N′-tetramethyluroniumhexafluoro-phosphate)
THF tetrahydrofuran
MDAP mass-directed autopurification

Compounds were named using ACD/Name PRO 6.02 chemical naming software (Advanced Chemistry Development Inc., Toronto, Ontario, M5H2L3, Canada) with the stereochemical designations (5r,8r) and (5s,8s) being replaced, respectively, by the more widely used “trans” and “cis” designations.

### Analytical Equipment

Proton Magnetic Resonance (NMR) spectra were recorded either on Varian instruments at 300, 400, 500 or 600 MHz, or on Bruker instruments at 300 or 400 MHz. Chemical shifts are reported in ppm (δ) using the residual solvent line as internal standard. Splitting patterns are designated as: s, singlet; d, doublet; t, triplet; q, quartet; m, multiplet; b, broad. The NMR spectra were recorded at a temperature ranging from 25 to 90° C. When more than one conformer was detected the chemical shifts for the most abundant one are reported.

Mass spectra (MS) were taken on a 4 II triple quadrupole Mass Spectrometer (Micromass UK) or on a Agilent MSD 1100 Mass Spectrometer, operating in ES(+) and ES(−) ionization mode. The usage of this methodology is indicated by “MS”.

HPLC-Mass spectra (HPLC-MS) were taken on a Agilent LC/MSD 1100 Mass Spectrometer, operating in ES(+) and ES(−) ionization mode coupled with HPLC instrument Agilent 1100 Series [LC/MS-ES (+): analysis performed on a Supelcosil ABZ +Plus (33×4.6 mm, 3 m) (mobile phase: 100% [water+0.1% formic acid] for 1 min, then from 100% [water+0.1% formic acid] to 5% [water+0.1% formic acid] and 95% [acetonitrile] in 5 min, finally under these conditions for 2 min; T=40° C.; flow=1 mL/min; LC/MS-ES (−): analysis performed on a Supelcosil ABZ +Plus (33×4.6 mm, 3 m) (mobile phase: 100% [water+0.05% ammonia] for 1 min, then from 100% [water+0.05% ammonia] to 5% [water+0.05% ammonia] and 95% [acetonitrile] in 5 min, finally under these conditions for 2 min; T=40° C.; flow=1 mL/min]. In the mass spectra only one peak in the molecular ion cluster is reported. The usage of this methodology is indicated by “HPLC-MS 1” in the analytical characterization of the described compounds.

Alternatively, HPLC-MS measurements were carried out using a Platform LCZTM single quadrupole Mass Spectrometer (Micromass-Waters), coupled with an HPLC system Agilent 1100 Series. The experimental conditions were: column XBridge C18, (5 mm 4.6×50 mm), column temperature 30° C., mobile phase, A=water+0.1% TFA and B=MeCN, gradient, t=0 min 0% (B) to 60% (B) in 1.5 min to 95% (B) in 3.5 min lasting for 1.5 min (t=6.60 min 0% B stop time=7.0 min), flow rate 2 ml/min, DAD UV range 210 to 350 nm, MS ionisation mode, positive electrospray (ES+), MS range 110 to 1100 atomic mass unit. The usage of this methodology is indicated by “HPLC-MS 2” in the analytical characterization of the described compounds.

Total ion current (TIC) and DAD UV chromatographic traces together with MS and UV spectra associated with the peaks were taken also on a UPLC/MS Acquity™ system equipped with 2996 PDA detector and coupled to a Waters Micromass ZQ™ mass spectrometer operating in positive or negative electrospray ionisation mode. [LC/MS-ES (+/−): analyses performed using an Acquity™ UPLC BEH C18 column (50×21 mm, 1.7 μm particle size), column temperature 40° C. (mobile phase: A-water+0.1% formic acid/B−acetonitrile+0.075% formic acid, Flow rate: 1.0 mL/min, Gradient: t=0 min 3% B, t=0.05 min 6% B, t=0.57 min 70% B, t=1.4 min 99% B, t=1.45 min 3% B)]. The usage of this methodology is indicated by “UPLC-MS” in the analytic characterization of the described compounds.

For reactions involving microwave irradiation, a Personal Chemistry Emrys™ Optimizer was used.

Flash silica gel chromatography was carried out on silica gel 230-400 mesh (supplied by Merck AG Darmstadt, Germany) or over Varian Mega Be-Si pre-packed cartridges or over pre-packed Biotage silica cartridges.

SPE-SCX cartridges are ion exchange solid phase extraction columns by supplied by Varian. The eluent used with SPE-SCX cartridges is methanol followed by 2N ammonia solution in methanol.

In a number of preparations, purification was performed using either Biotage manual flash chromatography (Flash+) or automatic flash chromatography (Horizon) systems. All these instruments work with standard Biotage Silica cartridges.

SPE-Si cartridges are silica solid phase extraction columns supplied by Varian.

In a number of preparations, purification was performed on a Mass-Directed Autopurification (MDAP) system Fractionlynx™ equipped with Waters 2996 PDA detector and coupled with ZQ™ mass spectrometer (Waters) operating in positive and negative electrospray ionisation mode ES+, ES−. (mass range 100-1000)

A set of acidic as well as basic semi-preparative gradients have been used:
METHOD A: Chromatographic Acidic conditions for up to 30 mg of crude:
Column: 100×21.2 mm Supelcosil™ ABZ +Plus (5 μm particle size)
Mobile phase: A[water+0.1% formic acid]/B[acetonitrile+0.1% formic acid]
Flow rate: 20 mL/min
Gradient: 5% B for 1 min, 95% B in 9 min, 100% B in 3.5 min
METHOD B: Chromatographic Acidic conditions for up to 100 mg of crude:
Column: 150×30 mm XTerra Prep MS C18 (10 μm particle size)
Mobile phase: A[water+0.1% formic acid]/B [acetonitrile+0.1% formic acid]
Flow rate: 40 mL/min
Gradient: 1% B to 100% B in 7 min lasting for 7.5 min.
METHOD C: Chromatographic Basic conditions for up to 100 mg of crude
Column: 150×30 mm XTerra Prep MS C18 (10 μm particle size)
Mobile phase: A-water+10 mM ammonium carbonate (adjusted to pH 10 with ammonia)/B−acetonitrile
Flow rate: 40 mL/min
Gradient: 10% B for 0.5 min, 95% B in 12.5 min

All reactions were monitored by thin-layer chromatography on 0.25 mm E. Merck silica gel plates (60F-254), visualised with UV light, iodine, 5% ethanolic phosphomolybdic acid, ninhydrin solution or vanillin solution.

### Supporting Compounds and Intermediates

### Intermediate 1

### Ethyl 2-oxo-3-phenyl-1-oxa-3-azaspiro[4.5]decane-8-carboxylate

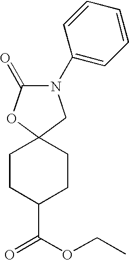

To a stirred solution of ethyl-4-hydroxy-4-({phenyl[(phenyloxy)carbonyl]amino}methyl)-cyclohexanecarboxylate (Intermediate 2) (127.3 mg, 0.320 mmol) in anhydrous toluene (2 ml) was added sodium hydride (60%, 19.21 mg). The reaction was stirred at room temperature overnight. The mixture was poured into water and extracted with EtOAc; the organic phase was dried on Na₂SO₄, filtered and evaporated in vacuo to give crude ethyl 2-oxo-3-phenyl-1-oxa-3-azaspiro[4.5]decane-8-carboxylate (93.2 mg), which was used without further purification; MS, m/z: 304 [M+H]⁺.

Another sample prepared using an analogous method showed the following NMR spectra

¹H NMR (400 MHz, CDCl₃): δ 1.23-1.33 (m, 3H) 1.55-1.69 (m, 2H) 1.86-2.21 (m, 6H) 2.32-2.41 (m, 1H) 3.71-3.74 (m, 2H) 4.11-4.22 (m, 2H) 7.11-7.18 (m, 1H) 7.35-7.43 (m, 2H) 7.50-7.59 (m, 2H). cis/trans 70:30

### Intermediate 2

### Ethyl 4-hydroxy-4-({phenyl[(phenyloxy)carbonyl]amino}methyl)cyclohexanecarboxylate

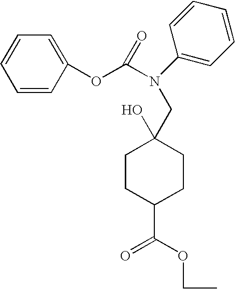

To a stirred solution of crude ethyl 4-hydroxy-4-[(phenylamino)-methyl]cyclohexanecarboxylate (Intermediate 3) (3.82 mmol) in DCM (10 ml) at 0° C. were added DIPEA (665 μl, 3.82 mmol) and phenyl chloroformate (480 μl, 3.82 mmol). The reaction was stirred at room temperature overnight. The mixture was poured into a saturated aqueous solution of NH₄Cl and extracted with DCM; the organic phase was dried on Na₂SO₄, filtered and evaporated in vacuo. The crude was purified by silica gel chromatography eluting with cyclohexane:EtOAc to give the title compound (127.3 mg, 8%); (Rf=0.48, Cyclohexane:EtOAc 7:3); MS: m/z 398 [M+H]⁺. Another batch of the same compound was prepared using an analogous method showed the following NMR spectra:

¹H NMR (400 MHz, CDCl₃): δ 1.18-1.36 (m, 5H) 1.47-1.94 (m, 7H) 2.14-2.22 (m, 1H) 2.39-2.46 (m, 1H) 3.82-3.90 (m, 1H) 3.91-3.95 (m, 1H) 4.05-4.19 (m, 2H) 7.03-7.14 (m, 2H) 7.16-7.24 (m, 1H) 7.28-7.44 (m, 6H). cis/trans 70:30

### Intermediate 3

### Ethyl 4-hydroxy-4-[(phenylamino)methyl]cyclohexanecarboxylate

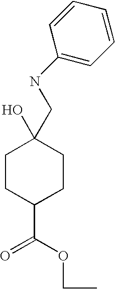

Ethyl 1-oxaspiro[2.5]octane-6-carboxylate (Intermediate 4 procedure 4a, 704.5 mg, 3.82 mmol) was dissolved in t-BuOH (4 ml) and aniline (697 μl, 7.65 mmol, Aldrich) was added. The reaction was stirred and heated at 150° C. under microwave irradiation for two 30 minute cycles. The mixture was poured into a saturated aqueous solution of NH₄Cl and extracted with ethyl acetate; the organic phase was dried on Na₂SO₄, filtered and evaporated in vacuo to give crude ethyl 4-hydroxy-4-[(phenylamino)methyl]cyclohexanecarboxylate (1.19 g), which was used without further purification.

Another batch of the same compound was prepared using an analogous method showed the following NMR spectra:

¹H NMR (400 MHz, CDCl₃): δ 1.22-1.30 (m, 3H) 1.36-2.02 (m, 9H) 2.23-2.34 (m, 1H) 2.45-2.54 (m, 1H) 3.09-3.13 (m, 1H) 3.16-3.21 (m, 1H) 4.10-4.20 (m, 2H) 6.65-6.77 (m, 3H) 7.14-7.24 (m, 2H). cis/trans 35:65

### Intermediate 4

### Ethyl 1-oxaspiro[2.5]octane-6-carboxylate

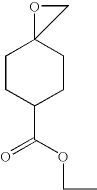

### Procedure 4a

To a mixture of trimethylsulfoxonium iodide and potassium tert-butoxide (as reported in Synthetic Communications, 33 (12), 2135-2143; 3.9 g, 11.76 mmol) was added a solution of ethyl 4-oxocyclohexanecarboxylate (1 g, 5.87 mmol, Aldrich) in DMSO (20 ml). The mixture was left to stir overnight at room temperature. The mixture was poured into water and extracted with diethyl ether; the organic phase was dried on Na₂SO₄, filtered and evaporated in vacuo to afford ethyl 1-oxaspiro[2.5]octane-6-carboxylate (704.5 mg, 65%), which was used without purification.

Another batch of the same compound prepared using an analogous method showed the following NMR spectra:

¹H NMR (400 MHz, CDCl₃): δ 1.20 (t, 3H) 1.27-1.49 (m, 2H) 1.63-2.04 (m, 6H) 2.26-2.28 (m, 1H) 2.49-2.59 (m, 2H) 4.06 (q, 2H) cis/trans 65:35

### Procedure 4b

A mixture of 2,8,9-triisobutyl-2,5,8,9-tetraaza-1-phosphabicyclo[3.3.3]undecane (commercially available, 1.14 ml, 3.94 mmol) and acetonitrile (15 ml) was added to a stirred suspension of trimethylsulphonium iodide (0.81 g, 3.97 mmol) and ethyl 4-oxocyclohexanecarboxylate (0.563 g, 3.31 mmol) at 0° C. The mixture was stirred at 0° C. for 30 minutes then allowed to warm to room temperature and stirred for a further 1 hour. The reaction mixture was concentrated under reduced pressure then diluted with diethyl ether. The resulting suspension was stirred for 30 minutes then filtered and the filter cake was washed with more diethyl ether. The combined ethereal phases were concentrated under reduced pressure and the residue was chromatographed on SiO₂ (Biotage 25M column) eluting with a gradient of 5%-15% EtOAc/cyclohexane to give a ˜60:40, trans:cis mixture of the title compound as a colourless oil (250 mg);

¹H NMR (400 MHz, CDCl₃): δ 1.27 (3H both isomers, t) 1.37-1.52 (2H both isomers, m), 1.68-2.14 (6H both isomers, m) 2.35-2.48 (1H both isomers, m), 2.62 (2H syn isomer, s), 2.65 (2H anti isomer, s), 4.16 (2H both isomers, q).

### Intermediate 5

### 2-Oxo-3-phenyl-1-oxa-3-azaspiro[4.5]decane-8-carbaldehyde

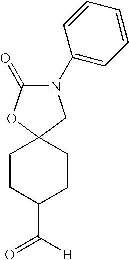

8-(Hydroxymethyl)-3-phenyl-1-oxa-3-azaspiro[4.5]decan-2-one (Intermediate 6, 72.5 mg, 0.277 mmol) was dissolved in dry DCM (3 ml) under nitrogen and Dess-Martin periodinane (141.38 mg, 0.33 mmol, Aldrich) was added in two portions and then the reaction was left at r.t. for 2 hours. The reaction was poured into a saturated solution of NaHCO₃ containing 5% of Na₂S₂O₃ (2.5 g) and extracted with DCM. The organic phase was dried on Na₂SO₄ and concentrated under vacuum to give the title compound which was used without further purification;

1H NMR (400 MHz, CDCl₃): δ 1.33-2.34 (m, 9H) 3.69-3.83 (m, 2H) 6.96-7.17 (m, 1H) 7.28-7.41 (m, 2H) 7.49-7.62 (m, 2H) 9.53-9.74 (m, 1H). cis/trans 85:15

### Intermediate 6

### 8-(Hydroxymethyl)-3-phenyl-1-oxa-3-azaspiro[4.5]decan-2-one

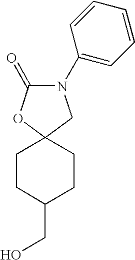

Ethyl 2-oxo-3-phenyl-1-oxa-3-azaspiro[4.5]decane-8-carboxylate (Intermediate 1, 110.0 mg, 0.363 mmol) was dissolved in anhydrous THF (2 ml) under nitrogen and was cooled to 0° C. At this temperature a solution of LiAlH₄ (1M, 272 μl, 0.272 mmol) was added drop by drop and then the reaction was left to warm to r.t. The reaction was diluted with Et₂O and two spatulas of Na₂SO₄ decahydrate were added portionwise; the mixture was left to stir overnight at r.t. The reaction was filtered, washing with Et₂O; the filtrate was concentrated under vacuum. The crude was purified by flash silica gel chromatography to give the title compound (72.5 mg, 76.5%);

¹H NMR (500 MHz, CDCl₃): δ 1.39 (3H, t), 1.49-1.66 (5H, m), 1.75-1.84 (2H, m), 2.10-2.16 (2H, m), 3.49-3.57 (21H, m), 3.73 (2H, s), 7.13 (1H, t), 7.38 (2H, t), 7.54 (2H, d);

MS, m/z: 262 [M+H]⁺

### Intermediate 7

### (Trans)-2-Oxo-3-phenyl-1-oxa-3-azaspiro[4.5]decane-8-carbaldehyde

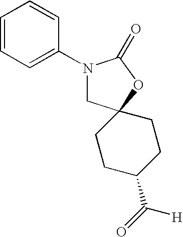

Dess-Martin periodinane (150 mg, 0.35 mmol) was added to a stirred solution of (trans)-8-(hydroxymethyl)-3-phenyl-1-oxa-3-azaspiro[4.5]decan-2-one (Intermediate 8, 76 mg, 0.29 mmol) in dichloromethane (3 ml) at room temperature. The resulting mixture was stirred for 1 hour then quenched with saturated aqueous solutions of sodium sulphite (0.5 ml) and sodium hydrogen carbonate (4 ml). The reaction mixture was stirred for 5 minutes then filtered through a hydrophobic frit (PhaseSep cartridge). The organic phase was shaken with more saturated sodium hydrogen carbonate solution (4 ml) and filtered through a hydrophobic frit (PhaseSep cartridge). The organic phase was concentrated under reduced pressure and the residue was chromatographed on SiO₂ eluting with a gradient of 30-50% EtOAc/cyclohexane to give the title compound as a white solid (60 mg);

¹H NMR (400 MHz, CDCl₃): δ 1.82 (4H, m), 1.92 (2H, m), 2.15 (2H, m), 2.52 (1H, m), 3.72 (2H, s), 7.15 (1H, t), 7.39 (2H, t), 7.53 (2H, d), 9.74 (1H, s).

### Intermediate 8

### (Trans)-8-(hydroxymethyl)-3-phenyl-1-oxa-3-azaspiro[4.5]decan-2-one

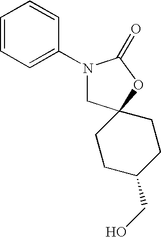

A solution of lithium aluminium hydride (1.0M in THF, 0.39 ml, 0.39 mmol) was added dropwise to a stirred solution of 1,1-dimethylethyl (trans)-2-oxo-3-phenyl-1-oxa-3-azaspiro[4.5]decane-8-carboxylate (Intermediate 9, 173 mg, 0.522 mmol) in THF (10 ml) at −20° C. The resulting mixture was allowed to stir and warm to 10° C. over a period of 1 hour. Further lithium aluminium hydride solution (1.0M in THF, 0.20 ml, 0.20 mmol) was added and the reaction mixture was allowed to warm to room temperature. The mixture was stirred for 30 minutes at room temperature then diluted with diethyl ether (20 ml) and quenched with a few drops of water. Sodium sulphate (1 g) was added and the mixture was stirred vigorously for 30 minutes then filtered. The filtrate was evaporated under reduced pressure and the residue was chromatographed on SiO₂ eluting with a gradient of 30-80% EtOAc/cyclohexane to give the title compound as a white solid (79 mg);

¹H NMR (400 MHz, CDCl₃): δ 1.17 (2H, m), 1.42 (1H, brs), 1.63 (1H, m), 1.85 (2H, dt), 1.99 (4H, m), 3.56 (2H, d), 3.79 (2H, s), 7.13 (1H, t), 7.38 (2H, t), 7.57 (2H, d).

### Intermediate 9

### 1,1-Dimethylethyl (trans)-2-oxo-3-phenyl-1-oxa-3-azaspiro[4.5]decane-8-carboxylate

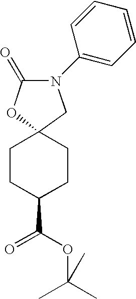

To a stirred mixture of (cis)-2-oxo-3-phenyl-1-oxa-3-azaspiro[4.5]decane-8-carboxylic acid (Intermediate 10, 0.38 g, 1.4 mmol), dimethyl formamide (0.1 ml) and tetrahydrofuran (8 ml) in a round-bottomed flask was added dropwise phosphorus oxychloride (0.15 ml, 1.6 mmol). The mixture was heated to 40° C. and stirred 2 hours. During this time, tetramethyl-ethylenediamine (0.73 ml, 4.8 mmol), tertiary butanol (0.20 ml, 2.1 mmol), lithium chloride (61 mg, 1.4 mmol) and tetrahydrofuran (2 ml) were stirred together in a separate vial. The flask was cooled to room temperature and the contents of the vial were added dropwise to the stirred solution of the intermediate acid chloride in the flask. The mixture was heated to 35° C. and stirred for 18 hours. The mixture was diluted with water and extracted twice with ethyl acetate. The combined organic extracts were washed (water, dilute hydrochloric acid, water), filtered through a hydrophobic membrane and concentrated under vacuum to give the crude product (0.47 g). The crude product was purified by flash column chromatography (silica gel; cyclohexane-ethyl acetate, 10:1); the fractions containing only the faster-running isomer were combined and concentrated under vacuum to give the title compound as a viscous oil which crystallised on standing (0.185 g, 40%);

¹H NMR (400 MHz, CDCl₃): δ 7.53 (2H, d), 7.34 (2H, t), 7.09 (1H, t), 3.73 (2H, s), 2.37 (1H, m), 2.08-1.99 (2H, m), 1.96-1.88 (2H, m), 1.87-1.78 (2H, m), 1.72-1.61 (2H, m) and 1.44 (9H, s);

UPLC-MS: 0.85 min, m/z 331 [M+H]+

### Intermediate 10

### (cis)-2-Oxo-3-phenyl-1-oxa-3-azaspiro[4.5]decane-8-carboxylic acid

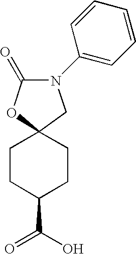

To a stirred solution of ethyl (cis)-2-oxo-3-phenyl-1-oxa-3-azaspiro[4.5]decane-8-carboxylate (Intermediate 11, 0.45 g, 1.5 mmol) in methanol (10 ml) was added dropwise a solution of lithium hydroxide (0.18 g) in water (2 ml). The mixture was stirred 1 hour then left to stand for 18 hours. The mixture was acidified with dilute hydrochloric acid (1 M) and extracted twice with ethyl acetate. The combined organic extracts were washed with water, filtered through a hydrophobic membrane and concentrated under vacuum to give the title compound (0.393 g, 96%) as a white solid.

¹H NMR (400 MHz, CDCl₃): δ 7.54 (2H, d), 7.38 (2H, t), 7.14 (1H, t), 3.75 (2H, s), 2.43 (1H, m), 2.19 (1H, m), 2.16 (1H, m), 2.08 (1H, m), 2.06-1.98 (3H, m) and 1.65 (2H, m); UPLC-MS: 0.62 min, m/z 274 [M−H].

### Intermediates 11 and 12

### Ethyl (cis)-2-oxo-3-phenyl-1-oxa-3-azaspiro[4.5]decane-8-carboxylate (Intermediate 11) and Ethyl (trans)-2-oxo-3-phenyl-1-oxa-3-azaspiro[4.5]decane-8-carboxylate (Intermediate 12)

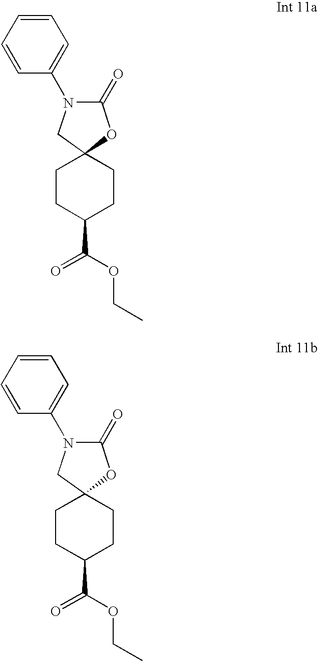

### Procedure 11a

Ethyl 4-hydroxy-4-[(phenylamino)methyl]cyclohexanecarboxylate (prepared in a similar fashion to Intermediate 3, 190.5 mg, 0.68 mmol) was dissolved in anhydrous DCM (10 ml) and was cooled to −50° C. under nitrogen. At this temperature TEA (189.38 μl, 1.36 mmol) and triphosgene (100.691 mg, 0.34 mmol) were added. The reaction was stirred at −78° C. for 2.5 hours. More triphosgene (100.0 mg, 0.337 mmol) was added, and the mixture was stirred for a further 2 hours (until complete). The reaction was treated with a saturated solution of NH₄Cl and was extracted with DCM; the organic phase was dried on Na₂SO₄, filtered and concentrated under vacuum to give a residue (175 mg), which was purified by flash silica gel chromatography (compound Rf=0.27, cyclohexane:EtOAc 7:3). After purification, two separated isomers were obtained: isomer 1 (Intermediate 12, 32.9 mg) and isomer 2 (Intermediate 11, 113.2 mg). The first corresponds to the trans and the second to the cis isomer;

Isomer 1 (trans), Intermediate 12:

¹H NMR (500 MHz, CDCl₃): δ 7.55 (2H, d), 7.38 (2H, t), 7.14 (1H, t), 4.16 (2H, q), 3.78 (2H, s), 2.46-2.57 (1H, m), 2.04-2.17 (2H, m), 1.84-2.02 (4H, m), 1.70-1.81 (2H, m), 1.28 (3H, t); MS: m/z 304 [M+H]+

Isomer 2 (cis), Intermediate 11:

¹H NMR (500 MHz, CDCl₃): δ 7.54 (2H, d), 7.38 (2H, t), 7.14 (1H, t), 4.15 (2H, q), 3.74 (2H, s), 2.30-2.42 (1H, m), 2.15 (2H, d), 1.91-2.09 (4H, m), 1.58-1.69 (2H, td), 1.28 (3H, t); MS: m/z 304[M+H]+.

### Procedure 11b

In a round bottom flask ethyl (trans)-2-oxo-1-oxa-3-azaspiro[4.5]decane-8-carboxylate (prepared in a similar fashion to Intermediate 15, 0.21 g, 0.924 mmol) was dissolved in toluene (2.1 ml). Iodobenzene (0.207 ml, 1.848 mmol), cesium carbonate (0.753 g, 2.310 mmol), copper(I) iodide (8.80 mg, 0.046 mmol) and trans-1,2-diaminocyclohexane (0.011 ml, 0.092 mmol) were added and the mixture was stirred at 80° C. overnight (overall 24 hours). The mixture was allowed to cool to room temperature and partitioned between water (20 ml) and ethyl acetate (2×20 ml). The combined organics were washed (water), filtered through a Phase Separator filter and concentrated under vacuum.

The crude was purified by column chromatography (silica gel; cyclohexane/ethyl acetate, 1:0 to 10:1 to 6:1, stepped gradient) to give Intermediate 13 (0.165 g, 59%) and Intermediate 12 (0.017 g, 7%).

### Intermediate 13:

¹H NMR (400 MHz, CDCl₃): δ 7.56 (2H, d), 7.39 (2H, t), 7.15 (1H, t), 4.17 (2H, q), 3.78 (2H, s), 2.48-2.57 (1H, m), 2.07-2.18 (2H, m), 1.85-2.03 (4H, m), 1.70-1.83 (2H, m), 1.29 (3H, t); UPLC-MS: 0.75 min, m/z 304 [M+H]+.

### Intermediate 12:

¹H NMR (400 MHz, CDCl₃): δ 7.55 (2H, d), 7.39 (2H, t), 7.15 (1H, t), 4.17 (2H, q), 3.75 (2H, s), 2.32-2.43 (1H, m), 2.12-2-22 (2H, m), 1.90-2.10 (4H, m), 1.58-1.70 (2H, m), 1.29 (3H, t) UPLC-MS: 0.74 min, m/z 304 [M+H]+.

### Intermediate 13

### 4-(3-Methyl-2-pyridinyl)-1,3-thiazol-2-amine

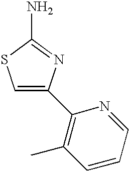

1-(3-Methyl-2-pyridinyl)ethanone (0.343 g, 2.54 mmol, available on the market), thiourea (0.042 g, 0.55 mmol) and iodine (0.103 g, 0.40 mmol) were dissolved in 1,4-dioxane (6 mL) and the mixture was stirred at 100° C. for 3 hours. Further portions of thiourea (0.021 g, 0.275 mmol) and iodine (0.05 g, 0.20 mmol) were added and the mixture was stirred at 100° C. for another 4 hours. Saturated aqueous NaHCO₃ solution was added and the mixture was extracted with DCM. The organic phase was washed with aqueous sodium thiosulfate solution and water. The organic extract was concentrated under vacuum to give a residue. The residue was purified by silica gel chromatography eluting with cyclohexane:EtOAc 100:0 to 60:40 to give the title compound as a brown solid (198 mg, 40%);

¹H NMR (400 MHz, CDCl₃): δ 2.54 (s, 3H) 4.92-5.12 (br. s, 2H) 6.92-6.94 (br. s, 1H) 7.11-7.18 (m, 1H) 7.51-7.59 (m, 1H) 8.47-8.55 (m, 1H).

### Intermediate 14

### 3-Bromo-2-fluoropyridine

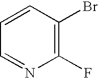

3-bromo-2-nitropyridine (1 g, 4.93 mmol) in N,N-dimethylformamide (10 ml) was added of tertrabutylammonium fluoride (8.96 ml, 9.85 mmol) and the solution was stirred at room temperature for 5 h. The dark red-brown reaction mixture was poured into 50 ml of a 1:1 mixture of water and EtOAc. The organic layer was washed twice with water and brine. The extracts were dried over Na₂SO₄, filtered and concentrated. The residue was purified by silica column 25M Biotage eluting with a mixture cyclohexane/EtOAc and the desired product elutes at 25% EtOAc to afford 313 mg of 3-bromo-2-fluoropyridine.

¹H-NMR (400 MHz, DMSO-d6): δ 8.16 (1H, d), 7.95-8.04 (1H, m), 7.08-7.14 (1H, m).

### Intermediates 15 and 16

Ethyl (trans)-2-oxo-1-oxa-3-azaspiro[4.5]decane-8-carboxylate (Intermediate 15) and ethyl (cis)-2-oxo-1-oxa-3-azaspiro[4.5]decane-8-carboxylate (Intermediate 16)

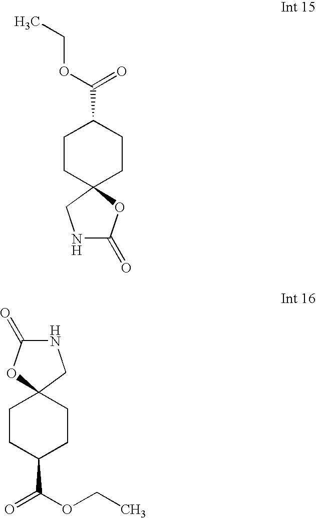

Potassium tert-butoxide (23.14 g, 206 mmol) was added portionwise to a stirred solution of ethyl carbamate (27.6 g, 309 mmol) in DMF (200 ml) at room temperature. The resulting cloudy mixture was stirred for 1 hour then a solution of ethyl 1-oxaspiro[2.5]octane-6-carboxylate (prepared in a similar fashion to intermediate 4 procedure 4b, 19 g, 103 mmol) in DMF (50 ml) was added. The reaction mixture was heated to 130° C. overnight (˜18 hours). Cool and dilute with saturated NaCl solution (20 ml) and extract with AcOEt (4×100 mL). The combined organic layers were dried (Na₂SO₄), filtered and concentrated to a pale yellow oil. The residue was purified via Biotage (cyclohexane:AcOEt starting from 1:1 to AcOEt pure; 65M column) to give Intermediate 15 (8.24 g) and intermediate 16 (4.36 g);

### Intermediate 15

¹H-NMR (400 MHz, CDCl₃): δ 5.39 (1H, brs), 4.15 (2H, q), 3.37 (2H, s), 2.47 (1H, sept), 2.01-2.11 (2H, m), 1.80-1.95 (4H, m), 1.62-1.74 (2H, m), 1.27 (3H, t).

### Intermediate 16.

¹H-NMR (400 MHz, CDCl₃): δ 5.27 (1H, brs), 4.15 (2H, q), 3.32 (2H, s), 2.28-2.37 (1H, m), 2.13 (2H, brd), 1.85-2.05 (4H, m), 1.53 (2H, td), 1.27 (3H, t).

### Intermediate 17

### (Trans)-8-(hydroxymethyl)-1-oxa-3-azaspiro[4.5]decan-2-one

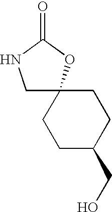

Lithium aluminium hydride (1.0M in THF, 22.00 ml, 22.00 mmol) was added to ethyl (trans)-2-oxo-1-oxa-3-azaspiro[4.5]decane-8-carboxylate (Intermediate 15, 2500 mg, 11.00 mmol) dissolved in tetrahydrofuran (THF) (50 ml) cooled to 0° C. Evolution of gas was observed adding first equivalent. The resulting mixture was allowed to warm up to room temperature. Na₂SO₄×10 (20 g) was added at −20° C. and left on standing for 1 h, allowing to warm up to room temperature. The resulting mixture was filtered washing with dichloromethane (500 ml) and dichloromethane/MeOH 90/10 (150 ml). Solvents were removed affording the title product as a colourless solid (2.4 g).

¹H-NMR (400 MHz, DMSO-d6): δ 4.60 (1H, brs), 3.11-3.27 (4H, m), 1.65-1.80 (4H, m), 1.51 (2H, td), 1.29-1.41 (1H, m), 0.90-1.04 (2H, m); UPLC-MS: 0.35 min, 186 [M+H]+.

### Intermediate 18

### (Trans)-2-oxo-1-oxa-3-azaspiro[4.5]decane-8-carbaldehyde

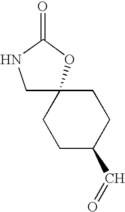

(Trans)-8-(hydroxymethyl)-1-oxa-3-azaspiro[4.5]decan-2-one (Intermediate 17, 1.2 g, 5.51 mmol) and PS—IBX amide (11.01 g, 11.01 mmol) were shaken in dichloromethane (100 ml) at room temperature for 24 h. A further 1.0 equiv. of PS—IBX amide was added and the reaction left for a further 24 h. The reaction was filtered washing with plenty of dichloromethane (500 ml). The collected organic phases were concentrated affording ca 1.3 g of crude oil. This was purified with Biotage SP1, over a 25M Silica cartridge pre-conditioned with 100% EtOAc, eluting with EtOAc (100%). The title compound (240 mg) was recovered as a colourless solid.

¹H-NMR (400 MHz, CDCl₃): δ 9.73 (1H, s), 5.34 (1H, brs), 3.32 (2H, s), 2.48 (1H, pentet), 2.06-2.15 (2H, m), 1.88-1.96 (2H, m), 1.71-1.82 (4H, m).

Alternatively, Intermediate 18 may be prepared in a similar fashion to the preparation of Intermediate 7 replacing (trans)-8-(hydroxymethyl)-3-phenyl-1-oxa-3-azaspiro[4.5]decan-2-one with (trans)-8-(hydroxymethyl)-1-oxa-3-azaspiro[4.5]decan-2-one (Intermediate 17). 1H NMR (400 MHz, DMSO-d6): δ 1.44-1.57 (m, 2H) 1.61-1.77 (m, 4H) 1.84-1.94 (m, 2H) 2.34-2.42 (m, 1H) 2.49-2.52 (m, 1H) 3.20 (d, 2H) 9.60 (d, 1H); UPLC-MS: 0.38 min, 184 [M+H]+

### Intermediate 19

### (Trans)-8-({[1-(2-fluorophenyl)-1H-pyrazol-3-yl]amino}methyl)-1-oxa-3-azaspiro[4.5]decan-2-one

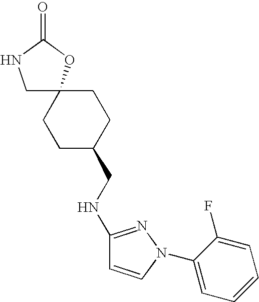

(Trans)-2-oxo-1-oxa-3-azaspiro[4.5]decane-8-carbaldehyde (Intermediate 18, 240 mg, 1.310 mmol), 1-(2-fluorophenyl)-1H-pyrazol-3-amine (232 mg, 1.310 mmol) (prepared according to the procedure described in J. Org. Chem. 2005, 70, 922) and titanium(IV) isopropoxide (0.768 ml, 2.62 mmol) were stirred in dichloromethane (2 ml) at room temperature overnight.

Then, sodium borohydride (149 mg, 3.93 mmol) and ethanol (2 ml) were added (caution, H₂). The resulting mixture was stirred for a further 5 h and then diluted with dichloromethane (150 ml) and quenched with sat NaHCO₃ (3 ml), then filtered over a filter tube. Solvent was removed and the resulting crude (450 mg) was purified with Biotage SP1, over a 25M KP—NH cartridge, eluting with 100% EtOAc to afford the title compound as colourless solid (350 mg).

¹H-NMR (400 MHz, CDCl₃): δ 7.82-7.87 (2H, m), 7.11-7.24 (3H, m), 5.95 (1H, brs), 5.82 (1H, d), 3.98 (1H, brs), 3.39 (2H, s), 3.15 (2H, d), 1.94-2.03 (4H, m), 1.65-1.85 (3H, m), 1.03-1.18 (2H, m); UPLC-MS: 0.67 min, 345 [M+H]+.

### Intermediate 20

### Ethyl (trans)-3-(1-methyl-1H-pyrazol-3-yl)-2-oxo-1-oxa-3-azaspiro[4.5]decane-8-carboxylate

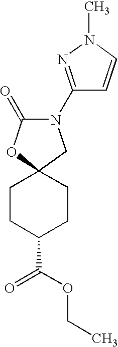

Ethyl (trans)-2-oxo-1-oxa-3-azaspiro[4.5]decane-8-carboxylate (Intermediate 15, 200 mg, 0.880 mmol), 3-iodo-1-methyl-1H-pyrazole (0.177 ml, 1.760 mmol), N,N′-dimethyl-1,2-ethanediamine (0.028 ml, 0.264 mmol), copper(I) iodide (50.3 mg, 0.264 mmol) and potassium carbonate (438 mg, 3.17 mmol) were suspended in 1,4-dioxane (8 ml). The mixture was irradiated in a microwave at 130° C. twice for 30 minutes then at 150° C. twice for 30 minutes. The reaction mixture was diluted with ethyl acetate (100 ml) and washed with water (20 ml), 0.25M aqueous hydrogenchloride (25 ml), saturated aqueous sodium hydrogencarbonate (25 ml) and brine (25 ml). The organic phase was dried (sodium sulfate), filtered and evaporated. The crude was chromatographed on silica gel eluting with cyclohexane/ethyl acetate 9/1 to 3/7. The desired product eluted at cyclohexane/ethyl acetate: 1/1. 180 mg of the title compound were collected.

¹H-NMR (400 MHz, CDCl₃): δ 7.29 (1H, d), 6.64 (1H, d), 4.16 (2H, qua), 3.86 (2H, s), 3.83 (3H, s), 2.47 (1H, sept), 2.04-2.15 (2H, m), 1.92-2.01 (2H, m), 1.87 (2H, td), 1.68-1.81 (2H, m), 1.28 (3H, t); UPLC-MS: 0.63 min, 308 [M+H]+.

### Intermediate 21

### Ethyl (trans)-3-(1-methyl-1H-pyrazol-3-yl)-2-oxo-1-oxa-3-azaspiro[4.5]decane-8-carboxylate

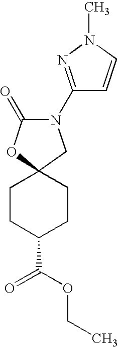

Ethyl (trans)-2-oxo-1-oxa-3-azaspiro[4.5]decane-8-carboxylate (Intermediate 15, 100 mg, 0.440 mmol), 3-iodo-1-methyl-1H-pyrazole (0.088 ml, 0.880 mmol), N,N′-dimethyl-1,2-ethanediamine (0.014 ml, 0.132 mmol), copper(I) iodide (25.1 mg, 0.132 mmol) and potassium carbonate (219 mg, 1.584 mmol) were suspended in 1,4-dioxane (4 ml). The mixture was irradiated in a microwave at 130° C. twice for 30 minutes then at 150° C. twice for 30 minutes. During the last cycle some epimerisation was noted (ca 2-3% as judged by UPLC-MS analysis). The reaction mixture was diluted with ethyl acetate (70 ml) and washed with water (20 ml), 0.25M aqueous hydrogen chloride (20 ml), saturated aqueous sodium hydrogencarbonate (20 ml) and brine (20 ml). The organic phase was dried (sodium sulfate), filtered and evaporated. The crude was chromatographed on silica gel eluting with cyclohexane/ethyl acetate 9/1 to 3/7. The desired product eluted at cyclohexane/ethyl acetate: 1/1.108 mg of the title compound were collected.

¹H-NMR (400 MHz, CDCl₃): δ 7.29 (1H, d), 6.64 (1H, d), 4.16 (2H, qua), 3.86 (2H, s), 3.83 (3H, s), 2.47 (1H, sept), 2.04-2.15 (2H, m), 1.92-2.01 (2H, m), 1.87 (2H, td), 1.68-1.81 (2H, m), 1.28 (3H, t); UPLC-MS: 0.63 min, 308 [M+H]+.

### Intermediate 22

### (Trans)-8-(hydroxymethyl)-3-(1-methyl-1H-pyrazol-3-yl)-1-oxa-3-azaspiro[4.5]decan-2-one

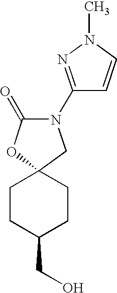

Ethyl (trans)-3-(1-methyl-1H-pyrazol-3-yl)-2-oxo-1-oxa-3-azaspiro[4.5]decane-8-carboxylate (Intermediate 20 and Intermediate 21, 284 mg, 0.924 mmol) was dissolved in tetrahydrofuran (6.6 ml) and cooled to −78° C. whereupon lithium aluminum hydride (0.924 ml, 0.924 mmol) was added dropwise. The mixture was allowed to warm to −40° C. and stirred for 1 hour at this temperature. The reaction was quenched with sodium sulfate decahydrate and diluted with diethyl ether. After stirring for 2 hours the suspension was filtered and the residue washed with dichloromethane (3×20 ml). The filtrate was evaporated and the crude chromatographed on silica gel eluting with dichloromethane/methanol:97/3 to 9/1.235 mg of the title compound were collected.

¹H-NMR (400 MHz, CDCl₃): δ 7.29 (1H, d), 6.66 (1H, d), 3.87 (2H, s), 3.83 (3H, s), 3.52 (2H, d), 1.89-2.04 (4H, m), 1.84 (2H, td), 1.54-1.67 (1H, m), 1.41 (1H, brs), 1.18 (2H, quad); UPLC-MS: 0.48 min, 266 [M+H]+.

### Intermediate 23

### (Trans)-3-(1-methyl-1H-pyrazol-3-yl)-2-oxo-1-oxa-3-azaspiro[4.5]decane-8-carbaldehyde

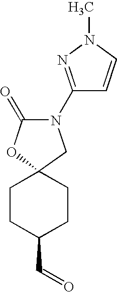

(Trans)-8-(hydroxymethyl)-3-(1-methyl-1H-pyrazol-3-yl)-1-oxa-3-azaspiro[4.5]decan-2-one (Intermediate 22, 230 mg, 0.867 mmol) was dissolved in dichloromethane (9.0 ml) and cooled with an ice-bath. Dess-Martin periodinane (441 mg, 1.040 mmol) was added in portions. The mixture was stirred for 2 hours while warming to 15° C. The mixture was diluted with dichloromethane (70 ml) and washed with saturated aqueous sodium hydrogencarbonate (25 ml) and brine (25 ml). The organic phase was passed through a hydrophobic PTFE frit and evaporated. The crude was taken up in dichloromethane and filtered in order to remove insoluble particles. The filtrate was evaporated and the obtained oil chromatographed on silica gel (Isolute) eluting with dichloromethane/ethyl acetate: 5/1. 183 mg of the title compound were collected.

¹H-NMR (400 MHz, CDCl₃): δ 9.73 (1H, s), 7.29 (1H, d), 6.64 (1H, d), 3.82 (3H, s), 3.80 (2H, s), 3.52 (2H, d), 2.42-2.51 (1H, m), 2.08-2.19 (2H, m), 1.88-1.98 (2H, m), 1.73-1.88 (4H, m); UPLC-MS: 0.54 min, 264 [M+H]+.

### Intermediate 24

### Ethyl (trans)-2-oxo-3-(2-pyridinyl)-1-oxa-3-azaspiro[4.5]decane-8-carboxylate

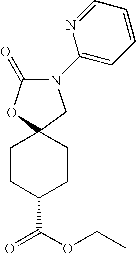

Ethyl (trans)-2-oxo-1-oxa-3-azaspiro[4.5]decane-8-carboxylate (prepared in a similar fashion to Intermediate 15, 700 mg, 3.08 mmol) was dissolved in 7 ml of toluene and 2-iodopyridine (1263 mg, 6.16 mmol), copper(I) iodide (29.3 mg, 0.154 mmol), (+/−)-trans-1,2-diaminocyclohexane (0.037 ml, 0.308 mol) and cesium carbonate (2509 mg, 7.70 mmol) were added and the mixture was heated at 80° C. and stirred vigorously for 18 h under a nitrogen atmosphere in a sealed tube. The mixture was cooled to room temperature and partitioned between water (70 ml) and ethyl acetate (2×100 ml). The combined organic extracts were washed (dilute hydrochloric acid, water), dried over Na₂SO₄, filtered and concentrated under vacuum. The crude was purified with SP1 silica gel column eluting with cyclohexane/ethyl acetate (93:7 to 50:50 gradient) to give the title compound (823.7 mg, 97% yield).

¹H NMR (500 MHz, CDCl₃): δ 8.33 (1H, d), 8.25 (1H, d), 7.68-74 (1H, m), 7.04 (1H, dd), 4.16 (2H, q), 4.03 (2H, s), 2.44-2.52 (1H, m), 2.04-2.15 (2H, m), 1.94-2.02 (2H, m), 1.84-1.92 (2H, m), 1.72-1.83 (2H, m), 1.28 (3H, t); UPLC-MS: 0.74 min, 305[M+H]⁺

### Intermediate 25

### (Trans)-8-(hydroxymethyl)-3-(2-pyridinyl)-1-oxa-3-azaspiro[4.5]decan-2-one

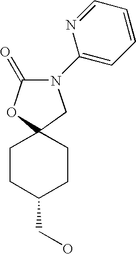

The title compound was made in a similar fashion to the preparation of Intermediate 22 using ethyl (trans)-2-oxo-3-(2-pyridinyl)-1-oxa-3-azaspiro[4.5]decane-8-carboxylate (Intermediate 24, 820 mg, 2.69 mmol) to give the title compound (416.8 mg, 59% yield). 1H NMR (400 MHz, CHLOROFORM-d): δ 8.29-8.25 (1H, m), 8.20 (1H, td), 7.66 (1H, td), 7.01-6.97 (1H, m), 4.00-3.98 (2H, m), 3.48-3.43 (2H, m), 2.48-2.43 (1H, m), 1.99-1.86 (4H, m), 1.84-1.73 (2H, m), 1.61-1.50 (1H, m), 1.109-1.09 (2H, m).

### Intermediate 26

### (Trans)-2-oxo-3-(2-pyridinyl)-1-oxa-3-azaspiro[4.5]decane-8-carbaldehyde

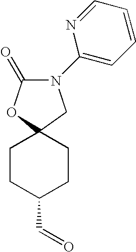

To a solution of (trans)-8-(hydroxymethyl)-3-(2-pyridinyl)-1-oxa-3-azaspiro[4.5]decan-2-one (Intermediate 25, 252 mg, 0.961 mmol) in DCM 45 ml, were added TPAP (42.9 mg, 0.122 mmol) and NMO (169 mg, 1.441 mmol) sequentially and the reaction mixture left at rt under stirring until the complete disappearance of the starting material as monitored by TLC (Cy:AcOEt 1:1, R_{f}=0.49). The reaction was diluted with DCM 10 ml and filtered through celite, the crude was purified on a Biotage 12M column of silica with SP1 eluting with cyclohexane/AcOEt 1:1 to give the title compound (133.7 mg, ˜54% yield).

1H NMR (400 MHz, CDCl₃): δ 9.72 (1H, s), 8.33-8.27 (1H, m), 8.23 (1H, td), 7.73-7.67 (1H, m) 7.06-7.00 (1H, m), 3.96 (2H, s), 2.51-2.43 (1H, m), 2.18-2.08 (2H, m), 1.97-1.72 (6H, m).

Alternatively, Intermediate 26 may be prepared in a similar fashion to the preparation of Intermediate 7 replacing (trans)-8-(hydroxymethyl)-3-phenyl-1-oxa-3-azaspiro[4.5]decan-2-one with (trans)-8-(hydroxymethyl)-3-(2-pyridinyl)-1-oxa-3-azaspiro[4.5]decan-2-one (Intermediate 25).

### Intermediate 27

### Ethyl (trans)-3-(4-fluorophenyl)-2-oxo-1-oxa-3-azaspiro[4.5]decane-8-carboxylate

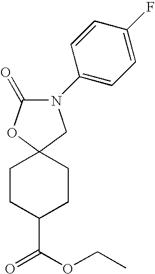

A 6.2:1 mixture of trans- and cis-Ethyl 2-oxo-1-oxa-3-azaspiro[4.5]decane-8-carboxylate (prepared in a similar fashion to Intermediates 15 and 16, 1.2 g, 5.28 mmol) was dissolved in 12.5 ml of toluene. 1-Bromo-4-fluorobenzene (0.924 g, 5.28 mmol), copper(I) iodide (0.050 g, 0.264 mmol), (+/−)-trans-1,2-diaminocyclohexane (0.063 ml, 0.528 mmol) and cesium carbonate (3.44 g, 10.56 mmol) were added. Then the mixture was heated at 150° C. for 30 minutes under microwave irradiation. Further 1-bromo-4-fluorobenzene (0.380 ml), copper(I) iodide (46 mg), (+/−)-trans-1,2-diaminocyclohexane (0.042 ml) and cesium carbonate (884 mg) were added and the mixture was subjected to another 3 cycles of microwave irradiation microwave (150° C. for 30 minutes).

The reaction was poured into water (50 ml) and extracted with AcOEt (2×80 ml), the organic phase washed with HCl 1 M (50 ml) then dried over Na₂SO₄ and concentrated under reduced pressure. The crude was purified on silica gel Flash 25M column with Biotage SP1, the column was eluted with cyclohexane/AcOEt from 80:20 to pure AcOEt. To give the title compound in a mixture with its cis-isomer (173.4 mg). This mixture was purified on a 12M column of silica gel using Biotage SP1 and eluting with cyclohexane/AcOEt (TLC 7:3, Rf=0.40 to afford the title compound (104 mg, 60% yield)

1H NMR (400 MHz, CDCl₃): δ 1.22-129 (t, 3H) 1.69-1.80 (m, 2H) 1.81-1.99 (m, 42H) 2.03-2.14 (m, 2H) 2.45-2.54 (m, 1H) 3.72-3.72 (m, 2H) 4.10-4.18 (m, 2H) 7.01-7.08 (m, 2H) 7.45-7.52 (m, 2H); UPLC-MS: 0.78 min, 322 [M+H]⁺

### Intermediate 28

### (Trans)-3-(4-fluorophenyl)-8-(hydroxymethyl)-1-oxa-3-azaspiro[4.5]decan-2-one

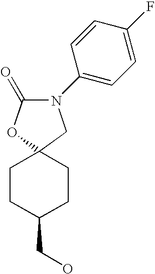

Ethyl (trans)-3-(4-fluorophenyl)-2-oxo-1-oxa-3-azaspiro[4.5]decane-8-carboxylate (Intermediate 27, 104 mg, 0.324 mmol) was dissolved into 10 ml of dry THF and the solution was cooled to −78° C. At this temperature LiAlH₄ (1.0M, 0.324 ml, 0.324 mmol) was added drop by drop under a nitrogen atmosphere. The reaction was diluted with Et₂O, 15 gr of Na₂SO₄.10H₂O were added and the reaction left to reach r.t.

The salts were filtered and washed with Et₂O. The organic phase was concentrated under vacuo to give 93 mg of crude title compound which was used without further purification. 1H NMR (400 MHz, CDCl₃) δ 1.13-1.23 (m, 1H) 1.56-1.66 (m, 1H) 1.67-1.73 (m, 2H) 1.75-1.89 (m, 2H) 1.92-2.04 (m, 3H) 3.54 (d, 2H) 3.67-3.72 (m, 1H) 3.76-3.79 (m, 2H) 7.04-7.10 (m, 2H) 7.49-7.55 (m, 2H).

### Intermediate 29

### (Trans)-3-(4-fluorophenyl)-2-oxo-1-oxa-3-azaspiro[4.5]decane-8-carbaldehyde

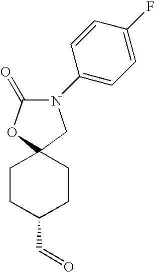

The title compound was made in a similar fashion to the preparation of Intermediate 7 using (trans)-3-(4-fluorophenyl)-8-(hydroxymethyl)-1-oxa-3-azaspiro[4.5]decan-2-one (Intermediate 28, 93 mg, 0.333 mmol) to give the title compound (35.8 mg, 37% yield)

1H NMR (400 MHz, CDCl₃): δ 1.74-1.90 (m, 4H) 1.90-2.02 (m, 2H) 2.08-2.27 (m, 2H) 2.47-2.58 (m, 1H) 3.69 (s, 2H) 7.08 (t, 2H) 7.48-7.52 (dd, 2H) 9.75 (s, 1H); UPLC-MS: 0.68 min, 278 [M+H]⁺

### Intermediate 30

### Ethyl (trans)-3-(2-fluorophenyl)-2-oxo-1-oxa-3-azaspiro[4.5]decane-8-carboxylate

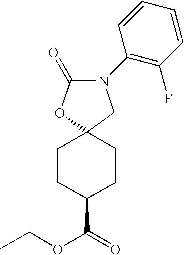

In a sealed reaction tube (miniblock XT 12 position), to a solution of 1-fluoro-2-iodobenzene and ethyl (trans)-2-oxo-1-oxa-3-azaspiro[4.5]decane-8-carboxylate (prepared in a similar fashion to Intermediate 15, 1 g, 4.40 mmol) in anhydrous dioxane (15 ml), CuI (42 mg, 0.220 mmol), trans-cyclohexanediamine (50 mg, 0.440 mmol) and K3PO4 (1.868 g, 8.80 mmol) were added. The mixture was heated under nitrogen flow at 115° C. for 1 hour. The mixture was stirred for further 30 minutes, then additional amounts of reagents (CuI, trans-cyclohexanediamine, 1-fluoro-2-iodobenzene 0.5 eq each) were added and heating continued for a further hour. The mixture was diluted with ethyl acetate, washed with water and the organic was back-extracted with EtOAc. The combined organic phases were dried (Na₂SO₄), filtered and concentrated under vacuum to give a residue which was purified by silica gel chromatography (40M+ column, eluent Cy-EtOAc 75:25) to afforded the title compound as a pale yellow oil (1.16 g). 1H NMR (400 MHz, CDCl₃) δ 1.29 (t, 3H) 1.70-1.83 (m, 2H) 1.89-2.05 (m, 4H) 2.05-2.19 (m, 2H) 2.48-2.57 (m, 1H) 3.81 (s, 2H) 4.17 (q, 2H) 7.11-7.32 (m, 3H) 7.56 (td, 1H); UPLC-MS: 0.73 minutes, 322 [M+H]⁺.

### Intermediate 31

### (Trans)-3-(2-fluorophenyl)-8-(hydroxymethyl)-1-oxa-3-azaspiro[4.5]decan-2-one

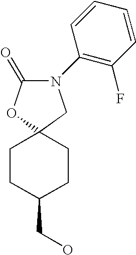

The title compound was made in a similar fashion to the preparation of Intermediate 22 using ethyl (trans)-3-(2-fluorophenyl)-2-oxo-1-oxa-3-azaspiro[4.5]decane-8-carboxylate (Intermediate 30, 300 mg, 0.934 mmol) to give the title compound (216 mg, 83% yield), 1H NMR (400 MHz, CDCl₃) δ 1.03-1.18 (m, 2H) 1.52-1.66 (m, 1H) 1.78-1.98 (m, 5H) 1.99-2.10 (m, 2H) 3.48 (d, 2H) 3.80 (s, 2H) 7.08-7.29 (m, 3H) 7.53 (td, 1H); UPLC-MS: 0.57 min, 280 [M+H]⁺

### Intermediate 32

### (Trans)-3-(2-fluorophenyl)-2-oxo-1-oxa-3-azaspiro[4.5]decane-8-carbaldehyde

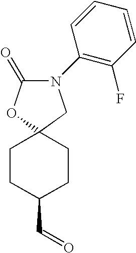

The title compound was made in a similar fashion to the preparation of Intermediate 7 using (trans)-3-(2-fluorophenyl)-8-(hydroxymethyl)-1-oxa-3-azaspiro[4.5]decan-2-one (Intermediate 31, 216 mg, 0.773 mmol) without a chromatographic purification step to give the title compound (150 mg, 70% yield), 1H NMR (400 MHz, CDCl₃) δ 1.77-1.92 (m, 4H) 1.96-2.08 (m, 2H) 2.10-2.23 (m, 2H) 2.47-2.57 (m, 1H) 3.75 (s, 2H) 7.12-7.32 (m, 3H) 7.55 (td, 1H) 9.75 (s, 1H); UPLC-MS: 0.63 min, 278 [M+H]⁺

### Intermediate 33

### Ethyl (trans)-2-oxo-3-[5-(trifluoromethyl)-3-pyridinyl]-1-oxa-3-azaspiro[4.5]decane-8-carboxylate

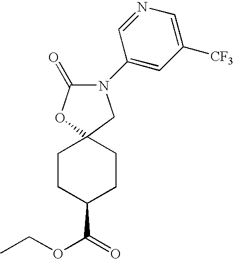

The title compound was made in a similar fashion to the preparation of Intermediate 20 replacing 3-iodo-1-methyl-1H-pyrazole with 3-bromo-5-(trifluoromethyl)pyridine (597 mg, 2.64 mmol) to give the title compound (213 mg). 1H-NMR (400 MHz, CDCl₃): δ 8.86 (1H, d), 8.67 (1H, d), 8.42 (1H, t), 4.19 (2H, qua), 3.84 (2H, s), 2.56 (1H, sept), 2.09-2.20 (2H, m), 1.89-2.05 (4H, m), 1.76-1.88 (2H, m), 1.30 (3H, t); UPLC-MS: 0.75 min, 373 [M+H]+.

### Intermediate 34

### (Trans)-8-(hydroxymethyl)-3-[5-(trifluoromethyl)-3-pyridinyl]-1-oxa-3-azaspiro[4.5]decan-2-one)

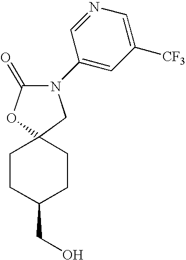

The title compound was made in a similar fashion to the preparation of Intermediate 22 using ethyl (trans)-2-oxo-3-[5-(trifluoromethyl)-3-pyridinyl]-1-oxa-3-azaspiro[4.5]decane-8-carboxylate (Intermediate 33, 190 mg, 0.510 mmol) to give the title compound (147 mg). 1H-NMR (400 MHz, CDCl₃): δ 8.89 (1H, d), 8.67 (1H, d), 8.43 (1H, t), 3.85 (2H, s), 3.59 (2H, t), 1.97-2.08 (4H, m), 1.86-1.97 (2H, m), 1.62-1.73 (1H, m), 1.42 (1H, t), 1.17-1.31 (2H, m); UPLC-MS: 0.62 min, 331 [M+H]+.

### Intermediate 35

### (Trans)-2-oxo-3-[5-(trifluoromethyl)-3-pyridinyl]-1-oxa-3-azaspiro[4.5]decane-8-carbaldehyde

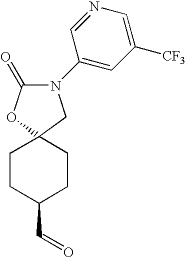

The title compound was made in a similar fashion to the preparation of Intermediate 23 using (trans)-8-(hydroxymethyl)-3-[5-(trifluoromethyl)-3-pyridinyl]-1-oxa-3-azaspiro[4.5]-decan-2-one (Intermediate 34, 140 mg, 0.424 mmol) to give the title compound (108 mg).

1H-NMR (400 MHz, CDCl₃): δ 9.77 (1H, s), 8.85 (1H, d), 8.67 (1H, d), 8.42 (1H, tt), 3.79 (2H, s), 2.57 (1H, quint), 2.12-2.23 (2H, m), 1.96-2.05 (2H, m), 1.80-1.96 (4H, m); UPLC-MS: 0.68 min, 329 [M+H]+.

### Intermediate 36

### Ethyl (trans)-2-oxo-3-(2-pyrazinyl)-1-oxa-3-azaspiro[4.5]decane-8-carboxylate

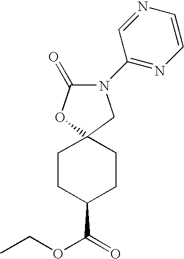

The title compound was made in a similar fashion to the preparation of Intermediate 20 replacing 3-iodo-1-methyl-1H-pyrazole with 2-chloropyrazine (0.236 ml, 2.64 mmol) to give the title compound (345 mg).

1H-NMR (400 MHz, CDCl₃): δ 9.60 (1H, dd), 8.34 (1H, d), 8.28 (1H, dd), 4.18 (2H, qua), 3.98 (2H, s), 2.52 (1H, sept), 2.07-2.18 (2H, m), 1.96-2.04 (2H, m), 1.87-1.96 (2H, m), 1.73-1.85 (2H, m), 1.30 (3H, t); UPLC-MS: 0.66 min, 306 [M+H]+.

### Intermediate 37

### (Trans)-8-(hydroxymethyl)-3-(2-pyrazinyl)-1-oxa-3-azaspiro[4.5]decan-2-one

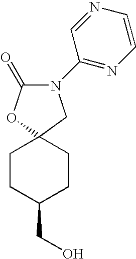

The title compound was made in a similar fashion to the preparation of Intermediate 22 using ethyl (trans)-2-oxo-3-(2-pyrazinyl)-1-oxa-3-azaspiro[4.5]decane-8-carboxylate (Intermediate 36, 325 mg, 1.064 mmol) to give the title compound (152 mg). 1H-NMR (400 MHz, CDCl₃): δ 9.62 (1H, dd), 8.34 (1H, dd), 8.28 (1H, dd), 4.00 (2H, s), 3.55 (2H, t), 1.94-2.08 (4H, m), 1.82-1.94 (2H, m), 1.61-1.70 (1H, m), 1.40 (1H, t), 1.15-1.30 (2H, m); UPLC-MS: 0.49 min, 264 [M+H]+.

### Intermediate 38

### (Trans)-2-oxo-3-(2-pyrazinyl)-1-oxa-3-azaspiro[4.5]decane-8-carbaldehyde

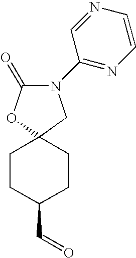

The title compound was made in a similar fashion to the preparation of Intermediate 23 using (trans)-8-(hydroxymethyl)-3-(2-pyrazinyl)-1-oxa-3-azaspiro[4.5]decan-2-one (Intermediate 37, 150 mg, 0.570 mmol) to give the title compound (91 mg).

1H-NMR (400 MHz, CDCl₃): δ 9.72 (1H, s), 8.60 (1H, dd), 8.34 (1H, d), 8.28 (1H, dd), 3.92 (2H, s), 2.47-2.55 (1H, m), 2.11-2.22 (2H, m), 1.92-2.02 (2H, m), 1.79-1.91 (4H, m); UPLC-MS: 0.57 min, 262 [M+H]+.

### Intermediate 39

### Ethyl (cis)-2-oxo-3-(3-pyridinyl)-1-oxa-3-azaspiro[4.5]decane-8-carboxylate

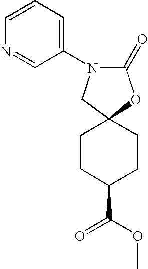

The title compound was made in a similar fashion to the preparation of Intermediate 30 replacing 1-fluoro-2-iodobenzene with 3-bromopyridine (250 mg, 1.584 mmol) and ethyl (trans)-2-oxo-1-oxa-3-azaspiro[4.5]decane-8-carboxylate with ethyl (cis)-2-oxo-1-oxa-3-azaspiro[4.5]decane-8-carboxylate (prepared in a similar fashion to Intermediate 16, 300 mg, 1.32 mmol) to give the title compound (290 mg).

1H NMR (400 MHz, CDCl₃): δ 8.55 (d, 1H), 8.40 (dd, 1H), 8.26 (dq, 1H), 7.36-7.31 (m, 1H), 4.17 (q, 2H), 3.78 (s, 2H), 2.43-2.33 (m, 1H), 2.22-1.15 (m, 8H), 1.28 (t, 3H); UPLC-MS: 0.53 min, 305 [M+H]+.

### Intermediate 40

### (Cis)-8-(hydroxymethyl)-3-(3-pyridinyl)-1-oxa-3-azaspiro[4.5]decan-2-one

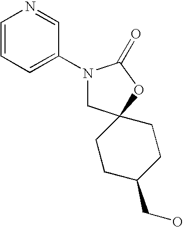

The title compound was made in a similar fashion to the preparation of Intermediate 22 using ethyl (cis)-2-oxo-3-(3-pyridinyl)-1-oxa-3-azaspiro[4.5]decane-8-carboxylate (Intermediate 39, 150 mg, 0.493 mmol) to give the title compound (121 mg).

¹H NMR (400 MHz, CDCl₃): δ 8.56 (d, 1H), 8.34 (d, 1H), 8.16 (dq, 1H), 7.32-7.28 (m, 1), 3.74 (s, 2H), 3.51 (d, 2H), 2.76 (br s, 1H), 2.21-1.38 (m, 9H). UPLC-MS: 0.38 min, 263 [M+H]+.

### Intermediate 41

### (Cis)-2-oxo-3-(3-pyridinyl)-1-oxa-3-azaspiro[4.5]decane-8-carbaldehyde

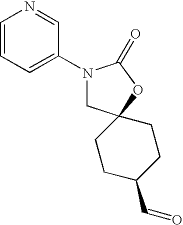

The title compound was made in a similar fashion to the preparation of Intermediate 23 using (cis)-8-(hydroxymethyl)-3-(3-pyridinyl)-1-oxa-3-azaspiro[4.5]decan-2-one (Intermediate 40, 121 mg, 0.461 mmol) to give the title compound (110 mg).

¹H NMR (400 MHz, CDCl₃): δ 9.67 (d, 1H), 8.55 (d, 1H), 8.42 (dd, 1H), 8.26 (dq, 1H), 7.34 (ddd, 1H), 3.80 (s, 2H), 2.38-2.27 (m, 1H), 2.25-0.81 (m, 8H). UPLC-MS: 0.40 min, 261 [M+H]+.

### Intermediate 42

### Ethyl (trans)-2-oxo-3-(3-pyridinyl)-1-oxa-3-azaspiro[4.5]decane-8-carboxylate

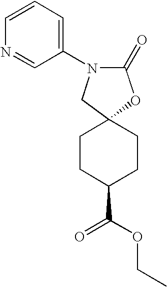

3-bromopyridine (209 mg, 1.320 mmol), ethyl (trans)-2-oxo-1-oxa-3-azaspiro[4.5]decane-8-carboxylate (prepared in a similar fashion to Intermediate 15, 300 mg, 1.320 mmol), (trans)-diaminocyclohexane (15.07 mg, 0.132 mmol) and 1,4-dioxane (20 ml) were collected into a 20 ml microwave vial and stirred at 150° C. for 30 min and then at 160° C. for 2 h 30 min under microwave irradiation. The reaction mixture was rinsed with DCM (100 ml) and washed with water (2×20 ml), then dried and concentrated under vacuum. The resulting crude was purified with Biotage SP1, over a 25M KP—NH cartridge, with a gradient of Cyclohexane/EtOAc. The title compound was recovered as colourless solid (190 mg). ¹H NMR (400 MHz, CDCl₃): δ 8.61 (dd, 1H), 8.41 (dd, 1H), 8.22 (dq, 1H), 7.33 (ddd, 1H), 4.18 (q, 2H), 3.61 (s, 2H), 2.55 (m, 1H), 2.18-1.75 (m, 8H), 1.29 (t, 3H); UPLC-MS: 0.56 m, 305 [M+H]+.

The corresponding isomer ethyl (cis)-2-oxo-3-(3-pyridinyl)-1-oxa-3-azaspiro[4.5]decane-8-carboxylate (Intermediate 39) was also recovered from this reaction as a colourless solid (120 mg).

¹H NMR (400 MHz, CDCl₃): δ 8.55 (d, 1H), 8.40 (dd, 1H), 8.25 (dq, 1H), 7.32 (ddd, 1H), 4.17 (q, 2H), 3.71 (s, 2H), 2.38 (m, 1H), 2.21-1.93 (m, 6H), 1.71-1.61 (m, 2H), 1.28 (t, 3H).

### Intermediate 43

### (Trans)-8-(hydroxymethyl)-3-(3-pyridinyl)-1-oxa-3-azaspiro[4.5]decan-2-one

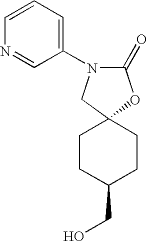

The title compound was made in a similar fashion to the preparation of Intermediate 22 using ethyl (trans)-2-oxo-3-(3-pyridinyl)-1-oxa-3-azaspiro[4.5]decane-8-carboxylate (prepared in a similar fashion to Intermediate 42, 1000 mg, 3.29 mmol) to give the title compound (600 mg).

1H NMR (400 MHz, CDCl₃): 8.62 (d, 1H), 8.41 (dd, 1H), 8.25 (dq, 1H), 7.34 (ddd, 1H), 3.84 (s, 2H), 3.58 (t, 2H), 2.07-1.15 (m, 9H); UPLC-MS: 0.38 min, 263 [M+H]+.

### Intermediate 44

### (Trans)-2-oxo-3-(3-pyridinyl)-1-oxa-3-azaspiro[4.5]decane-8-carbaldehyde

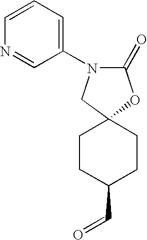

The title compound was made in a similar fashion to the preparation of Intermediate 23 using (trans)-8-(hydroxymethyl)-3-(3-pyridinyl)-1-oxa-3-azaspiro[4.5]decan-2-one (Intermediate 43, 100 mg, 0.381 mmol) to give the title compound (50 mg).

1H NMR (400 MHz, CDCl₃): 9.76 (s, 1H), 8.60 (d, 1H), 8.41 (dd, 1H), 8.21 (dq, 1H), 7.34 (ddd, 1H), 3.76 (s, 2H), 2.59-2.51 (m, 1H), 2.22-1.26 (m, 8H).

### Intermediate 45

### 1-(3-Fluoro-2-pyridinyl)ethanone

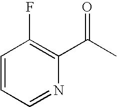

3-Fluoro-2-pyridinecarbonitrile (2 g, 16.38 mmol) was dissolved in dry diethyl ether (6 ml) and cooled to 0° C., then methylmagnesium bromide (6.55 ml, 19.66 mmol) as a solution in diethyl ether (12 ml) was added dropwise. After completion of the addition, the reaction was stirred for 16 hrs while being allowed to warm to rt. The mixture was quenched with 1M aq HCl (50 ml) and stirred for 2 hrs, then basified with NH4OH until pH 8. The solution was extracted with DCM (3×100 ml). The combined organics were dried (Na₂SO₄), filtered and evaporated to dryness to give 2.1 g of crude which was purified on silica gel column biotage 40 M eluting with a mixture cyclohexane/EtOAc 10/0 to 6/4 to give the title compound (1.4 g).

1H NMR (400 MHz, CDCl₃): δ 8.52 (dt, 1H), 7.58-7.49 (m, 2H), 2.73 (s, 3H); UPLC-MS: 0.47 min, 139 [M+H]+.

### Intermediate 46

### 2-Bromo-1-(3-fluoro-2-pyridinyl)ethanone

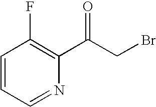

Chloro(trimethyl)silane (1.368 ml, 10.78 mmol) and triethylamine (2.99 ml, 21.56 mmol) were added to a solution of 1-(3-fluoro-2-pyridinyl)ethanone (Intermediate 45, 1.250 g, 8.98 mmol) in DMF (25 ml). The mixture was shaken at 60° C. for 23 hrs. The crude was left to warm to rt and DMF was stripped off by Biotage V10. The residue was dissolved in 100 ml of DCM and washed twice with cold NaHCO3. The organic phases were collected and DCM was evaporated. The crude obtained was dissolved in tetrahydrofuran (25.00 ml) and N-bromosuccinimide (1.599 g, 8.98 mmol) was added. The mixture was stirred at 0° C. for 1 h and then left to warm to rt and stirred for 94 hrs. THF was evaporated under reduced pressure and the crude was dissolved in DCM and washed twice with NaHCO3. The organic phases were collected and concentrated under reduced pressure to get 1.7 g of crude which was purified by silica column Biotage 40M eluting with a mixture DCM/Et2O (1/0 to 4/6) to give the title compound (490 mg).

1H NMR (400 MHz, CDCl₃): δ 8.54-8.51 (m, 1H), 7.62-7.56 (m, 2H), 4.76 (s, 2H); UPLC-MS: 0.61 min, 218, 220 [M+H]+.

### Intermediate 47

### 4-(3-fluoro-2-pyridinyl)-1,3-thiazol-2-amine

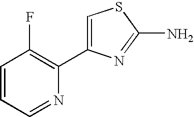

2-Bromo-1-(3-fluoro-2-pyridinyl)ethanone (Intermediate 46, 480 mg, 2.202 mmol) and thiourea (168 mg, 2.202 mmol) were dissolved in ethanol (20 ml) and stirred at 90° C. for 2 h. The ethanol was evaporated under reduced pressure and the crude obtained was dissolved in DCM and washed twice with NaHCO3. The organic phase was evaporated and since the crude obtained was purified by SCX ion exchange cartridge to give the title compound (320 mg, 1.639 mmol, 74.5% yield).

1H NMR (400 MHz, CDCl₃): δ 8.41 (d, 1H), 7.73 (dd, 1H), 7.44-7.35 (m, 1H), 7.19 (s, 1H), 7.14 (s, 1H); UPLC-MS: 0.39 min, 195 [M+H]+.

### Intermediate 48

### Ethyl (trans)-3-(2-methyl-3-pyridinyl)-2-oxo-1-oxa-3-azaspiro[4.5]decane-8-carboxylate

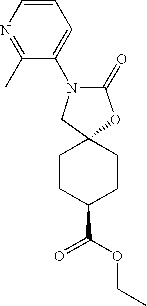

In a reaction tube (miniblock XT 24 position), to a solution of 3-bromo-2-methylpyridine (1.009 ml, 8.77 mmol) and ethyl (trans)-2-oxo-1-oxa-3-azaspiro[4.5]decane-8-carboxylate (prepared in a similar fashion to Intermediate 15, 1.66 g, 7.30 mmol) in anhydrous dioxane (22 ml), CuI (0.070 g, 0.365 mmol), trans-cyclohexanediamine (0.088 ml, 0.730 mmol) and K₃PO₄ (3.10 g, 14.61 mmol) were added. The mixture was heated under a nitrogen flow at 115° C. (external temperature) for 4 h. CuI (693 mg, 3.65 mmol) and trans-cyclohexanediamine (0.875 mL, 7.30 mmol) were added and the external temperature was raised to 130° C. (internal T=110° C.) and the mixture was stirred for an additional 2 h. After stirring at room temperature overnight the mixture was diluted with EtOAc and washed with water (2×20 mL). The aqueous layer was back-extracted with ethyl acetate and the combined organic layers were dried over Na₂SO₄, filtered and concentrated to obtain a yellow oil. Purification by a series of chromatographies (40M+ NH cartridge, elution in gradient with cyclohexane/EtOAc up to 100% followed by 25M+ NH column elution in gradient with cyclohexane/EtOAc up to 50% followed by 25M+ NH cartridge eluting in gradient with cyclohexane/EtOAc up to 50%) combining clean product fractions to give the title compound (478 mg).

¹H NMR (400 MHz, CDCl₃): δ 8.50 (dd, 1H), 7.58 (dd, 1H), 7.23 (dd, 1H), 4.17 (2H, q), 3.68 (s, 2H), 2.56 (s, 3H), 2.50-2.58 (m, 1H), 1.92-2.07 (m, 4H), 1.71-1.83 (m, 2H), 1.28 (t, 3H); UPLC-MS: 0.54 min, 319 [M+H]+.

### Intermediate 49

### (Trans)-8-(hydroxymethyl)-3-(2-methyl-3-pyridinyl)-1-oxa-3-azaspiro[4.5]decan-2-one

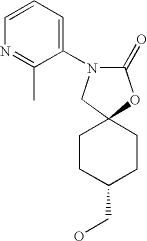

The title compound was made in a similar fashion to the preparation of Intermediate 22 using ethyl (trans)-3-(2-methyl-3-pyridinyl)-2-oxo-1-oxa-3-azaspiro[4.5]decane-8-carboxylate (this may be prepared as described for Intermediate 48, 180 mg, 0.565 mmol) to give the title compound as a colourless solid (115 mg).

¹H NMR (400 MHz, CDCl₃): δ 8.50 (dd, 1H), 7.58 (dd, 1H), 7.22 (dd, 1H), 3.71 (s, 2H), 3.53 (t, 2H), 2.56 (s, 3H), 2.15-1.38 (m, 9H), 1.15 (q, 2H); UPLC-MS: 0.37 min, 277 [M+H]+.

### Intermediate 50

### (Trans)-3-(2-methyl-3-pyridinyl)-2-oxo-1-oxa-3-azaspiro[4.5]decane-8-carbaldehyde

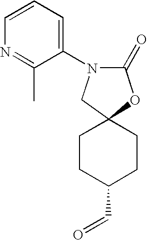

The title compound was made in a similar fashion to the preparation of Intermediate 23 using (trans)-8-(hydroxymethyl)-3-(2-methyl-3-pyridinyl)-1-oxa-3-azaspiro[4.5]decan-2-one (Intermediate 49, 115 mg, 0.416 mmol) to give the title compound (110 mg) as colourless solid.

¹H NMR (400 MHz, DMSO-d₆): δ 9.62 (s, 1H), 8.41 (dd, 1H), 7.80 (dd, 1H), 7.30 (dd, 1H), 3.76 (s, 1H), 2.40 (s, 3H), 2.00-1.50 (m, 8H); UPLC-MS: 0.41 min, 275 [M+H]+.

### Intermediate 51

### Ethyl (trans)-2-oxo-3-(5-pyrimidinyl)-1-oxa-3-azaspiro[4.5]decane-8-carboxylate

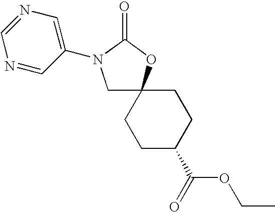

A 75/25 mixture of cis/trans ethyl 2-oxo-1-oxa-3-azaspiro[4.5]decane-8-carboxylate, ((prepared in a similar fashion to intermediates 15 and 16, 500 mg, 2.200 mmol), 5-bromopyrimidine (350 mg, 2.200 mmol), copper(I) iodide (41.9 mg, 0.220 mmol), potassium phosphate (1401 mg, 6.60 mmol), (trans)-diammino cyclohexane (50.2 mg, 0.440 mmol) were collected into a Carousel tube and then were suspended in 1,4-Dioxane (10 ml). The resulting mixture was stirred in a Stem Block apparatus at 130° C. for 24 h. The reaction mixture was rinsed with DCM (300 ml), washed with water (2×50 ml) filtering over separation tube. The resulting organic phase was concentrated in vacuo to afford 700 mg of crude material. This was purified with Biotage SP1, on a KP—NH 40M column, using a gradient of cyclohexane/EtOAc. The title compound was eluted with ca 30% EtOAc and recovered as colourless solid (130 mg).

¹H NMR (400 MHz, CDCl₃): δ 9.01 (s, 2H), 8.90 (s, 1H), 4.04 (q, 2H), 3.95 (m, 2H), 2.52-2.35 (m, 1H), 2.01-1.83 (m, 4H), 1.81-1.66 (m, 2H), 1.66-1.46 (m, 2H), 1.17 (t, 3H); UPLC-MS: 0.59 min, 306 [M+H]+.

Ethyl (cis)-2-oxo-3-(5-pyrimidinyl)-1-oxa-3-azaspiro[4.5]decane-8-carboxylate was eluted with ca 45% EtOAc and recovered as a colourless solid (170 mg).

¹H NMR (400 MHz, DMSO-d6): δ 9.03 (s, 2H), 9.02 (s, 1H), 4.18 (q, 2H), 3.76 (m, 2H), 2.47-2.35 (m, 1H), 2.24-1.94 (m, 4H), 1.81-1.66 (m, 1H), 1.78-1.65 (m, 2H), 1.29 (t, 3H). UPLC-MS: 0.58 min, 306 [M+H]+.

### Intermediate 52

### (Trans)-8-(hydroxymethyl)-3-(5-pyrimidinyl)-1-oxa-3-azaspiro[4.5]decan-2-one

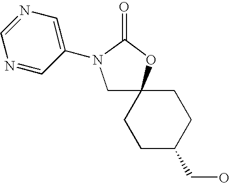

The title compound was made in a similar fashion to the preparation of Intermediate 22 using ethyl (trans)-2-oxo-3-(5-pyrimidinyl)-1-oxa-3-azaspiro[4.5]decane-8-carboxylate (Intermediate 51, 130 mg, 0.426 mmol) to give the title compound as a colourless solid (50 mg). ¹H NMR (400 MHz, CDCl₃): δ 9.05 (s, 2H), 9.02 (s, 1H), 3.61 (s, 2H), 3.59 (t, 2H), 2.08-1.87 (m, 6H), 1.71-1.61 (m, 1H), 1.30-1.15 (m, 2H). UPLC-MS: 0.43 min, 264 [M+H]+.

### Intermediate 53

### (Trans)-2-oxo-3-(5-pyrimidinyl)-1-oxa-3-azaspiro[4.5]decane-8-carbaldehyde

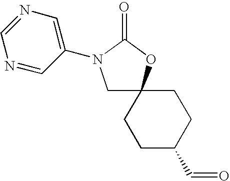

The title compound was made in a similar fashion to the preparation of Intermediate 23 using (trans)-8-(hydroxymethyl)-3-(5-pyrimidinyl)-1-oxa-3-azaspiro[4.5]decan-2-one (Intermediate 52, 50 mg, 0.190 mmol) to give the title compound as a colourless solid (40 mg).

¹H NMR (400 MHz, CDCl₃): δ 9.77 (s, 1H), 9.03 (s, 1H), 9.02 (s, 2H), 3.74 (s, 2H), 2.57 (quint, 1H), 2.23-1.80 (m, 8H); UPLC-MS: 0.45 min (broad peak), 262 [M+H]+.

### Intermediate 54

### Ethyl (cis)-2-oxo-3-(2-pyridinyl)-1-oxa-3-azaspiro[4.5]decane-8-carboxylate

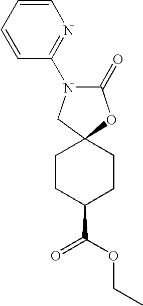

The title compound was made in a similar fashion to the preparation of Intermediate 24 replacing ethyl (trans)-2-oxo-1-oxa-3-azaspiro[4.5]decane-8-carboxylate with ethyl (cis)-2-oxo-1-oxa-3-azaspiro[4.5]decane-8-carboxylate and (prepared in a similar fashion to Intermediate 16, 5.71 g, 25.1 mmol) to give the title compound (4.24 g). ¹H NMR (400 MHz, CDCl₃): δ 1.28 (t, 3H) 1.60-1.71 (m, 2H) 1.91-2.08 (m, 4H) 2.10-2.19 (m, 2H) 2.33-2.44 (m, 1H) 4.17 (q, 2H) 7.04 (dd, 1H) 7.68-7.76 (m, 1H) 8.26 (d, 1H) 8.32 (dd, 1H); UPLC-MS: 0.71 min, 305 [M+H]+.

### Intermediate 55

Ethyl 8-methyl-2-oxo-3-(2-pyridinyl)-1-oxa-3-azaspiro[4.5]decane-8-carboxylate

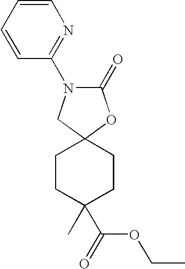

A THF solution of ethyl (cis)-2-oxo-3-(2-pyridinyl)-1-oxa-3-azaspiro[4.5]decane-8-carboxylate (this may be prepared as described for Intermediate 54, 502.7 mg, 1.65 mmol) and LDA (1.8M, 2.75 ml, 4.96 mmol) was cooled to −78° C. and stirred for 0.5 hour at that temperature. MeI (0.361 ml, 5.78 mmol) was added and the reaction was stirred for an additional 3 hours at −78° C. The reaction mixture was poured into water, extracted twice with ether and the organic layer dried over Na₂SO₄. Evaporation of the solvent gave a crude which was purified on silica gel column eluting with 10-50% EtOAc/cyclohexane to give the title compound (416.3 mg) as a mixture of two diastereoisomers with ˜70/30 ratio. 1H-NMR (500 MHz, CDCl₃): δ 8.32 (1H major isomer, d), 8.30 (1H minor isomer, d), 8.27 (1H major isomer, d), 8.24 (1H minor isomer, d), 7.67-7.74 (1H major isomer+1H minor isomer, m), 7.00-7.07 (1H major isomer+1H minor isomer, m), 4.15-4.23 (2H major isomer+2H minor isomer, m), 4.03 (2H major isomer, s), 3.97 (2H minor isomer, s), 2.27-2.35 (2H major isomer, m), 2.10-2.17 (2H minor isomer, m), 1.60-2.02 (4H major isomer+4H minor isomer, m), 1.38-1.48 (2H major isomer+2H minor isomer, m), 1.29 (3H major isomer+3H minor isomer, t), 1.24 (3H minor isomer, s), 1.23 (3H major isomer, s); UPLC-MS: 1.11, 319 [M+H]+ (major isomer) and 1.13 min, 319 [M+H]+ (minor isomer)

### Intermediates 56 and 57

### (Cis)-8-(hydroxymethyl)-8-methyl-3-(2-pyridinyl)-1-oxa-3-azaspiro[4.5]decan-2-one (Intermediate 56) and (Trans)-8-(hydroxymethyl)-8-methyl-3-(2-pyridinyl)-1-oxa-3-azaspiro[4.5]decan-2-one (Intermediate 57)

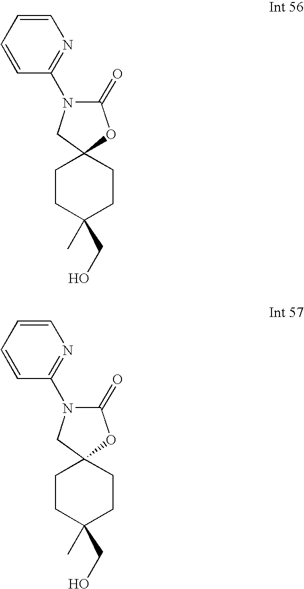

The title compounds were made in a similar fashion to the preparation of Intermediate 22 using ethyl 8-methyl-2-oxo-3-(2-pyridinyl)-1-oxa-3-azaspiro[4.5]decane-8-carboxylate (Intermediate 54, 300 mg, 0.942 mmol) but allowing the reaction to reach room temperature before quenching to give Intermediate 56 (200 mg, 0.651 mmol, 69.1%) and Intermediate 57 (60.4 mg, 0.214 mmol, 22.73%)

### Intermediate 56

1H-NMR (500 MHz, CDCl₃): δ 8.32 (1H, d), 8.26 (1H, d), 7.71 (1H, td), 7.03 (1H, dd), 3.99 (2H, s), 3.43 (2H, d), 1.93-2.01 (2H, m), 1.72-1.81 (4H, m), 1.34-1.39 (2H, m), 1.01 (3H, s); UPLC-MS: 0.84 min, 277 [M+H]+

### Intermediate 57

1H-NMR (500 MHz, CDCl₃): δ 8.32 (1H, d), 8.26 (1H, d), 7.72 (1H, td), 7.04 (1H, dd), 4.01 (2H, s), 3.42 (2H, d), 1.92-2.04 (2H, m), 1.73-1.84 (2H, m), 1.47-1.66 (4H, m), 1.02 (3H, s); UPLC-MS: 0.82 min, 277 [M+H]+

### Intermediate 58

### (Cis)-8-methyl-2-oxo-3-(2-pyridinyl)-1-oxa-3-azaspiro[4.5]decane-8-carbaldehyde

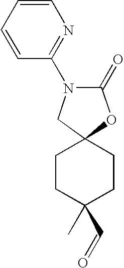

(Cis)-8-(hydroxymethyl)-8-methyl-3-(2-pyridinyl)-1-oxa-3-azaspiro[4.5]decan-2-one (Intermediate 56, 100 mg, 0.362 mmol) and PL_IBX amide resin were suspended in dry dichloromethane (10 ml) and shaken. The reaction was monitored by UPLC and resulted very slow. 3 equivalents of PL_IBX amide resin were added and after 5 days the reaction was filtered on a filter tube and washed with dichloromethane, then the organic phase was concentrated under vacuum to give 95 mg of crude which was purified on a silica gel cartridge eluting with cyclohexane:AcOEt (from 30 to 100% and with AcOEt:MeOH until 50%) to give the title compound (57 mg) as a white solid.

1H-NMR (400 MHz, CDCl₃): δ 9.51 (1H, s), 8.34 (1H, d), 8.27 (1H, d), 7.70-7.77 (1H, m), 7.07 (1H, dd), 4.05 (2H, s), 2.14-2.24 (4H, m), 1.92-2.02 (2H, m), 1.82-1.91 (2H, m), 1.41-1.51 (2H, m), 1.12 (3H, s); UPLC-MS: 0.63 min, 275 [M+H]+

### Intermediate 59

### (Trans)-8-methyl-2-oxo-3-(2-pyridinyl)-1-oxa-3-azaspiro[4.5]decane-8-carbaldehyde

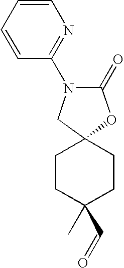

The title compound was made in a similar fashion to the preparation of Intermediate 23 using (trans)-8-(hydroxymethyl)-8-methyl-3-(2-pyridinyl)-1-oxa-3-azaspiro[4.5]decan-2-one (prepared in a similar fashion to Intermediate 57, 55 mg, 0.199 mmol) to give the title compound (32 mg) as a white solid. 1H-NMR (400 MHz, CDCl₃): δ 9.48 (1H, s), 8.32 (1H, dd), 8.23 (1H, d), 7.66-7.75 (1H, m), 6.99-7.08 (1H, m), 3.92 (2H, s), 1.95-2.05 (4H, m), 1.75-1.87 (2H, m), 1.59-1.71 (2H, m), 1.10 (3H, s); UPLC-MS: 0.69 min, 275 [M+H]+

### Intermediate 60

### Ethyl (trans)-3-(6-methyl-2-pyridinyl)-2-oxo-1-oxa-3-azaspiro[4.5]decane-8-carboxylate

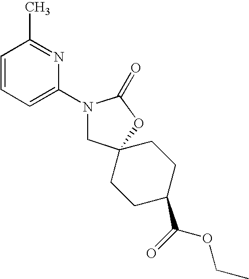

The title compound was made in a similar fashion to the preparation of Intermediate 51 replacing ethyl (cis/trans)-2-oxo-1-oxa-3-azaspiro[4.5]decane-8-carboxylate ethyl (trans)-2-oxo-1-oxa-3-azaspiro[4.5]decane-8-carboxylate (prepared in a similar fashion to Intermediate 15, 904 mg, 3.98 mmol) and 5-bromopyrimidine with 2-bromo-6-methylpyridine (0.543 ml, 4.77 mmol) to give the title compound as a white solid (222 mg, 17%). 1H NMR (400 MHz, CDCl₃) δ 1.29 (t, 3H) 1.73-1.94 (m, 4H) 1.94-2.03 (m, 2H) 2.12 (ddd, 2H) 2.45-2.55 (m, 4H) 4.03 (s, 2H) 4.18 (q, 2H) 6.89 (d, 1H) 7.59 (t, 1H) 8.03 (d, 1H) The epimer ethyl (cis)-3-(6-methyl-2-pyridinyl)-2-oxo-1-oxa-3-azaspiro[4.5]decane-8-carboxylate was also recovered from this reaction as a colourless oil (258 mg, 20%).

1H NMR (400 MHz, CDCl₃) δ 1.26-1.32 (t, 3H) 1.54-1.70 (m, 2H) 1.91-2.19 (m, 6H) 2.33-2.43 (m, 1H) 2.48 (s, 3H) 3.99 (s, 2H) 4.13-4.21 (q, 2H) 6.87-6.91 (d, 1H) 7.56-7.63 (t, 1H) 8.02-8.07 (d, 1H).

### Intermediate 61

### (Trans)-8-(hydroxymethyl)-3-(6-methyl-2-pyridinyl)-1-oxa-3-azaspiro[4.5]decan-2-one

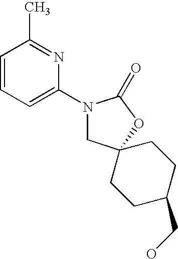

The title compound was made in a similar fashion to the preparation of Intermediate 22 using ethyl (trans)-3-(6-methyl-2-pyridinyl)-2-oxo-1-oxa-3-azaspiro[4.5]decane-8-carboxylate (Intermediate 60, 40.6 mg, 0.128 mmol) to give the title compound as uncolourless oil (40 mg, 0.127 mmol).

1H NMR (400 MHz, CDCl₃) δ 1.17-1.44 (m, 4H) 1.55-1.69 (m, 1H) 1.91-2.05 (m, 4H) 2.49 (s, 3H) 3.53-3.60 (t, 2H) 4.03 (s, 2H) 6.85-6.91 (d, 1H) 7.54-7.62 (t, 1H) 8.02-8.07 (d, 1H); UPLC-MS: 0.61 min, 277 [M+H]+.

### Intermediate 62

### (Trans)-3-(6-methyl-2-pyridinyl)-2-oxo-1-oxa-3-azaspiro[4.5]decane-8-carbaldehyde

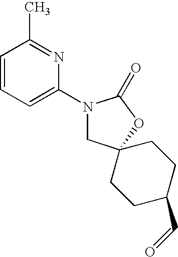

The title compound was made in a similar fashion to the preparation of Intermediate 23 using (trans)-8-(hydroxymethyl)-3-(6-methyl-2-pyridinyl)-1-oxa-3-azaspiro[4.5]decan-2-one (Intermediate 61, 36 mg, 0.130 mmol) to give the title compound (24 mg, 67%).

1H NMR (400 MHz, CDCl₃) δ 1.78-1.96 (m, 6H) 2.09-2.21 (m, 2H) 2.44-2.53 (m, 4H) 3.98 (s, 2H) 6.89 (d, 1H) 7.60 (t, 1H) 8.03 (d, 1H) 9.76 (s, 1H); UPLC-MS: 0.68 min, 275 [M+H]+.

### Intermediate 63

### Ethyl (trans)-3-(6-fluoro-2-pyridinyl)-2-oxo-1-oxa-3-azaspiro[4.5]decane-8-carboxylate

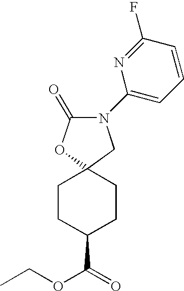

Sodium hydride (0.132 g, 3.30 mmol) was stirred in dry DMF (10 mL) under a nitrogen atmosphere. Ethyl (trans)-2-oxo-1-oxa-3-azaspiro[4.5]decane-8-carboxylate (prepared in a similar fashion to Intermediate 15, 0.75 g, 0.30 mmol) dissolved in dry DMF (10 mL) was slowly added at 0° C. A white suspension was formed and the mixture was left stirring while the temperature raised to r.t. (ca 30 min). Then 2,6-difluoropyridine (0.359 ml, 3.96 mmol) dissolved in dry DMF (1 mL) was added and the mixture was stirred at 50° C. for 1.5 hours. The mixture was cooled to room temperature. A few drops of water were added and the solvent was removed under reduced pressure to give a residue that was purified via Biotage SP1 (25+M silica column) eluting with cyclohexane/EtOAc 9:1 to 7:3. Product fractions were combined and evaporated under reduced pressure to give 90 mg of pure product and 180 mg of a mixture that was further purified by Biotage SP1 (25+M silica column) eluting with cyclohexane/EtOAc 9:1 to 7:3. Product fractions were combined and evaporated under reduced pressure, dissolved again in DCM, combined with the 90 mg batch from the first chromatography and evaporated under vacuo to afford the title compound (210 mg, 19%).

1H NMR (500 MHz, CDCl₃) δ 1.28 (t, 3H) 1.71-1.82 (m, 2H) 1.83-1.93 (m, 2H) 1.90-2.04 (m, 2H) 2.06-2.18 (m, 2H) 2.40-2.56 (m, 1H) 3.98 (s, 2H) 4.15 (q, 2H) 6.66 (dd, 1H) 7.78-7.84 (m, 1H) 8.12 (d, 1H); UPLC-MS: 0.79 min, 323 [M+H]+.

### Intermediate 64

### (Trans)-3-(6-fluoro-2-pyridinyl)-2-oxo-1-oxa-3-azaspiro[4.5]decane-8-carbaldehyde

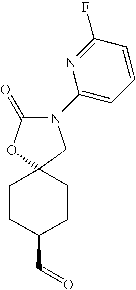

Ethyl (trans)-3-(6-fluoro-2-pyridinyl)-2-oxo-1-oxa-3-azaspiro[4.5]decane-8-carboxylate (Intermediate 63, 210 mg, 0.652 mmol) was dissolved in tetrahydrofuran (8 mL) under a nitrogen atmosphere. The mixture was cooled to −78° C. and lithium aluminium hydride (0.489 mL, 0.489 mmol, 1M in THF) was slowly added then the mixture was stirred at −78° C. for 2.5 hrs. The mixture was diluted with diethylether (10 mL), two spatula of sodium sulfate decahydrate were added and the mixture was vigorously stirred while the temperature raised to r.t. (ca 3 h). The precipitate was filtered off through a separatory tube washing with Et₂O and the mixture was evaporated under reduced pressure to give a crude that was purified by silica column chromatography (Biotage SP1, 12+M column) eluting with cyclohexane/EtOAc 100:0 to 70:30 to afford the title compound as a white solid (22 mg, 12%).

1H NMR (400 MHz, CDCl₃) δ 1.75-2.05 (m, 6H) 2.08-2.21 (m, 2H) 2.43-2.53 (m, 1H) 3.94 (s, 2H) 6.63-6.70 (m, 1H) 7.67-7.92 (m, 1H) 8.11 (m, 1H) 9.74 (s, 1H); UPLC-MS: 0.68 min, 279 [M+H]+.

The alcohol (trans)-3-(6-fluoro-2-pyridinyl)-8-(hydroxymethyl)-1-oxa-3-azaspiro[4.5]decan-2-one (74 mg, 40%) was also recovered as a white solid. UPLC-MS: 0.61 min, 281 [M+H]+.

### Intermediate 65

### (Trans)-8-(1H-1,2,3-benzotriazol-1-yl{[4-(2-pyridinyl)-1,3-thiazol-2-yl]amino}methyl)-3-(2-Pyridinyl)-1-oxa-3-azaspiro[4.5]decan-2-one

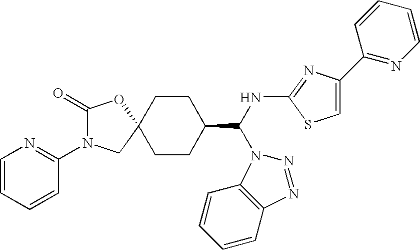

To a stirred solution of 4-(2-pyridinyl)-1,3-thiazol-2-amine (12.26 mg, 0.069 mmol) and 1H-1,2,3-benzotriazole (8.24 mg, 0.069 mmol) in dry toluene (2 ml) under nitrogen was added dropwise a solution of (trans)-2-oxo-3-(2-pyridinyl)-1-oxa-3-azaspiro[4.5]decane-8-carbaldehyde (this may be prepared as described for intermediate 26, 20 mg, 0.077 mmol) in toluene (2 ml). The mixture was stirred overnight. The mixture was heated to 100° C. and stirred 3 hours under a nitrogen atmosphere. The mixture was cooled to room temperature and cyclohexane (5 ml) was added. The mixture was stirred 10 minutes then filtered, washing with cyclohexane. The solid filter-cake was dried under vacuum. The filter was washed with ethyl acetate—the washings were combined and concentrated under vacuum to give the title compound (36 mg).

1H NMR (400 MHz, CDCl₃): δ 8.58 (1H, d), 8.35 (1H, d), 8.26 (1H, d), 8.08 (1H, d), 7.82-7.99 (2H, m), 7.70-7.78 (2H, m), 7.54 (1H, t), 7.45-7.52 (1H, m), 7.36-7.43 (1H, m), 7.16-7.24 (1H, m), 7.06 (1H, dd), 6.55 (1H, d), 4.10 (1H, d), 4.06 (1H, d), 2.62-2.75 (1H, m), 2.46 (1H, brd), 1.55-2.20 (6H, m), 1.21-1.40 (2H, m).

### Intermediate 66

### 5-fluoro-4-(2-pyridinyl)-1,3-thiazol-2-amine

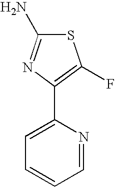

To a solution of 4-(2-pyridinyl)-1,3-thiazol-2-amine (1 g, 5.64 mmol) in DMF (20 ml) was added Selectfluor (2.231 g, 6.30 mmol) at 0° C. The resulting mixture was stirred at r.t. for 2 h. Then, it was rinsed with DCM and then washed with water twice. The organic phases were collected and DCM evaporated. The crude obtained was purified by KP—NH cartridge Biotage 40M eluting with a mixture of cyclohexane/EtOAc to give the title compound (34.5 mg).

¹H NMR (400 MHZ, CDCl₃): δ 8.69-8.75 (1H, d), 7.71-7.84 (2H, m), 7.18-7.25 (1H, m), 4.63-4.90 (2H, brs). UPLC-MS: 0.37 min, 196 [M+H]+

Compound having lower chemically purity was recovered and further purified with a silica gel column 40 M Biotage eluting with a gradient DCM/MeOH to give a further quantity of the title product (100 mg).

### Intermediate 67

### 4-Bromo-1-(tetrahydro-2H-pyran-2-yl)-1H-pyrazole

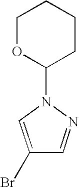

TFA (2.62 μl, 0.034 mmol) was added to a mixture of 4-bromo-1H-pyrazole (100 mg, 0.680 mmol) and 3,4-dihydro-2H-pyran (86 mg, 1.021 mmol) in a glass vial. The resulting mixture was shaken and heated to 80° C. for 16 hours. Cool reaction mixture to room temperature then partition between dichloromethane (5 ml) and dilute aqueous sodium hydroxide solution (1M, 2 ml). Filter through a hydrophobic frit (Phase Separator cartridge) washing with more dichloromethane and evaporate the combined organics under reduced pressure. The residue was purified via Biotage (10%-30% EtOAc/cyclohexane; 25M SiO2 column) to give the title compound (148 mg) as a colourless oil.

1H-NMR (400 MHz, CDCl₃): δ 7.66 (1H, s), 7.52 (1H, s), 5.33-5.42 (1H, m), 4.06 (1H, dd), 3.66-3.77 (1H, m), 1.97-2.15 (3H, m), 1.61-1.79 (3H, m); HPLC-MS: 2.00 min, 147 and 149 [M-C5H8O+H]+

### Intermediate 68

### 3-bromo-1-(tetrahydro-2H-pyran-2-yl)-1H-pyrazole

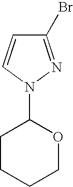

The title compound was made in a similar fashion to the preparation of Intermediate 67 using 3-bromo-1H-pyrazole (100 mg, 0.680 mmol) to give the title compound (121 mg). 1H-NMR (400 MHz, CDCl₃): δ 7.53 (1H, d), 6.34 (1H, d), 5.35 (1H, dd), 4.07 (1H, dd), 3.63-3.79 (1H, m), 1.97-2.23 (3H, m), 1.60-1.81 (3H, m); HPLC-MS: 1.93 min, 147 and 149 [M-C5H8O+H]+

### Intermediate 69

### (Trans)-8-({[1-(2-fluorophenyl)-1H-pyrazol-3-yl]amino}methyl)-3-[1-(tetrahydro-2H-pyran-2-yl)-1H-pyrazol-3-yl]-1-oxa-3-azaspiro[4.5]decan-2-one

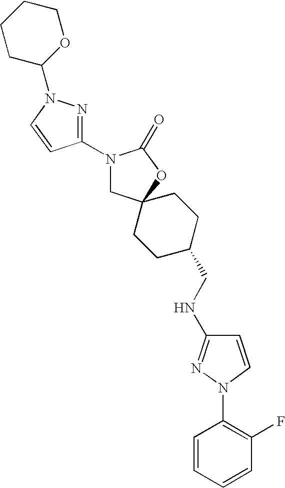

A mixture of 3-bromo-1-(tetrahydro-2H-pyran-2-yl)-1H-pyrazole (Intermediate 68, 53.7 mg, 0.232 mmol), (trans)-8-({[1-(2-fluorophenyl)-1H-pyrazol-3-yl]amino}methyl)-1-oxa-3-aza-spiro[4.5]decan-2-one (prepared in a similar fashion to Intermediate 19, 40 mg, 0.116 mmol), copper (I) iodide (22.12 mg, 0.116 mmol), (+/−)-(trans)-1,2-diaminocyclohexane (26.5 mg, 0.232 mmol) and potassium phosphate tribasic (123 mg, 0.581 mmol) in dioxane (3 ml) was sealed in a glass tube and shaken at 120° C. for 20 hours. Cool to room temperature and evaporate the solvent. Add dichloromethane (5 ml) to the residue and filter washing with more dichloromethane (2×1 ml). Wash the combined organic phases with pH3 citrate buffer solution (5 ml) then filter through a hydrophobic frit (Phase Separator cartridge) and evaporate. The residue was purified via Biotage (30%-100% EtOAc/cyclohexane; 12M NH column) to give the title compound (45 mg) as a colourless oil.

1H-NMR (400 MHz, CDCl₃): δ 7.82-7.93 (2H, m), 7.55 (1H, d), 7.11-7.26 (3H, m), 6.77 (1H, d), 5.83 (1H, d), 5.26 (1H, dd), 4.11 (1H, dd), 3.92 (2H, s), 3.72 (1H, td), 3.17 (2H, d), 1.92-2.22 (7H, m), 1.79-1.92 (2H, m), 1.56-1.79 (4H, m), 1.12-1.31 (2H, m); m/z 495 [M+H]+, 411 [M-C5H8O+H]+

### Intermediate 70

### (Trans)-8-({[1-(2-fluorophenyl)-1H-pyrazol-3-yl]amino}methyl)-3-[1-(tetrahydro-2H-pyran-2-yl)-1H-pyrazol-4-yl]-1-oxa-3-azaspiro[4.5]decan-2-one

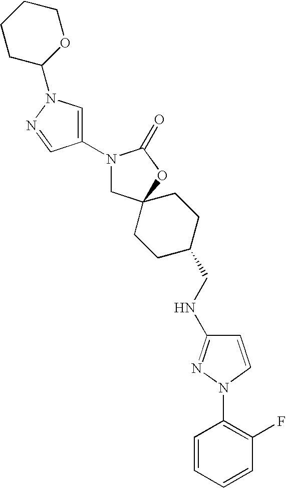

The title compound was made in a similar fashion to the preparation of Intermediate 69 replacing 3-bromo-1-(tetrahydro-2H-pyran-2-yl)-1H-pyrazole with 4-bromo-1-(tetrahydro-2H-pyran-2-yl)-1H-pyrazole (Intermediate 67, 40.3 mg, 0.174 mmol) to give the title compound (48 mg).

1H-NMR (400 MHz, CDCl₃): δ 7.94 (1H, s), 7.82-7.90 (2H, m), 7.55 (1H, s), 7.11-7.26 (3H, m), 5.83 (1H, d), 5.36 (1H, dd), 4.02-4.10 (1H, m), 3.64-3.75 (1H, m), 3.68 (2H, s), 3.19 (2H, d), 1.95-2.21 (7H, m), 1.87 (2H, td), 1.55-1.81 (4H, m), 1.12-1.26 (2H, m); HPLC-MS: 2.56 min, 411 [M-C5H8O+H]+

### Intermediate 71

### Ethyl 8-fluoro-2-oxo-3-(2-pyridinyl)-1-oxa-3-azaspiro[4.5]decane-8-carboxylate

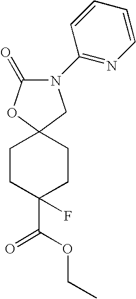

A THF (45 ml) solution of ethyl (trans)-2-oxo-3-(2-pyridinyl)-1-oxa-3-azaspiro[4.5]decane-8-carboxylate (this may be prepared as described for Intermediate 54, 500 mg, 1.643 mmol) and LDA (1.8 M, 2.74 ml, 4.93 mmol) was cooled to −78° C. and stirred for 0.5 hour at that temperature. N-Fluorobenzenesulfonimide (1036 mg, 3.29 mmol) was added and the reaction was stirred for an additional 3 hours at −78° C. The reaction mixture was poured into water, extracted twice with ether and the combined organic layers dried over Na₂SO₄. Evaporation of the solvent gave a crude which was purified on silica gel column eluting with cyclohexane/ethyl acetate (from 10 to 100) to afford the title compound (249.5 mg, 42% yield) as a mixture of two isomers in a ratio ˜60:40.

1H NMR (400 MHz, CDCl₃) δ 1.30-1.37 (m, 3H) 1.92-2.14 (m, 4H) 2.15-2.27 (m, 2H) 2.28-2.38 (m, 1H) 2.38-2.48 (m, 1H) 4.02 (s, 1H) 4.11 (s, 1H) 4.23-4.32 (m, 2H) 7.02-7.09 (m, 1H) 7.69-7.76 (m, 1H) 8.15-8.36 (m, 2H).

### Intermediates 72 and 73

### 8-Fluoro-8-(hydroxymethyl)-3-(2-pyridinyl)-1-oxa-3-azaspiro[4.5]decan-2-one (Intermediate 72) and 8-fluoro-8-(hydroxymethyl)-3-(2-pyridinyl)-1-oxa-3-azaspiro[4.5]decan-2-one (Intermediate 73)

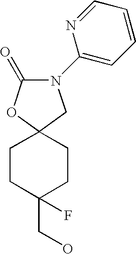

The title compounds were made in a similar fashion to the preparation of Intermediate 23 using ethyl 8-fluoro-2-oxo-3-(2-pyridinyl)-1-oxa-3-azaspiro[4.5]decane-8-carboxylate (prepared in a similar fashion to Intermediate 71, 315 mg, 0.977 mmol) (to give (136.3 mg, 47%) of the major isomer Intermediate 72 and (84.9 mg, 30%) of the minor isomer Intermediate 73.

### Intermediate 72

1H NMR (500 MHz, CDCl₃): δ 8.32 (1H, d), 8.24 (1H, d), 7.73 (1H, t), 7.09-7.01 (1H, m), 4.01 (2H, s), 3.64 (2H, dd), 2.15-1.79 (8H, m), 1.76 (1H, t); UPLC-MS: 0.65 min, 281 [M+H]+

### Intermediate 73

1H NMR (500 MHz, CDCl₃): δ 8.32 (1H, d), 8.27 (1H, d), 7.73 (1H, t), 7.10-7.02 (1H, m), 4.06 (2H, s), 3.64 (2H, dd), 2.30-2.10 (4H, m), 1.95-1.77 (2H, m), 1.77-1.53 (3H, m); UPLC-MS: 0.63 min, 281 [M+H]+

### Intermediate 74

### 8-Fluoro-2-oxo-3-(2-pyridinyl)-1-oxa-3-azaspiro[4.5]decane-8-carbaldehyde

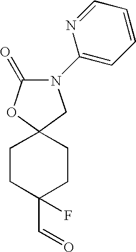

The title compound was made in a similar fashion to the preparation of Intermediate 23 using 8-fluoro-8-(hydroxymethyl)-3-(2-pyridinyl)-1-oxa-3-azaspiro[4.5]decan-2-one (Intermediate 72, 135 mg, 0.482 mmol) to give the title compound (116 mg, 69.2%).

1H NMR (500 MHz, CDCl₃): δ 9.75 (1H, d), 8.32 (1H, d), 8.24 (1H, d), 7.79-7.66 (1H, m), 7.10-7.01 (1H, m), 4.03 (2H, s), 2.40-1.69 (8H, m); UPLC-MS: 0.59 min, 279 [M+H]+ and 297 [M+H2O+H]+

### Intermediate 75

### 8-Fluoro-2-oxo-3-(2-pyridinyl)-1-oxa-3-azaspiro[4.5]decane-8-carbaldehyde

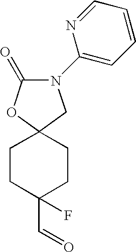

The title compound was made in a similar fashion to the preparation of Intermediate 23 using 8-fluoro-8-(hydroxymethyl)-3-(2-pyridinyl)-1-oxa-3-azaspiro[4.5]decan-2-one (Intermediate 73, 83 mg, 0.296 mmol) to give the title compound (50 mg, 57.6%).

1H NMR (500 MHz, CDCl₃): δ 9.82 (1H, d), 8.34 (1H, d), 8.26 (1H, d), 7.74 (1H, t), 7.11-7.03 (1H, m), 4.09 (2H, s), 2.32-1.76 (8H, m); UPLC-MS: 0.58 min, 279 [M+H]+ and 297 [M+H2O+H]+

### Intermediate 76

### 1-(2-Fluorophenyl)-4-iodo-1H-pyrazol-3-amine

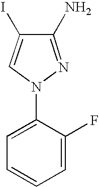

1-(2-fluorophenyl)-1H-pyrazol-3-amine (1 g, 5.64 mmol) (N2911-53-1) was dissolved in N,N-Dimethylformamide (20 mL). N-Iodosucciniimide (1.333 g, 5.93 mmol) was added and the mixture was stirred at r.t. for 3 h. DMF was removed under reduced pressure and the crude compound was dissolved in 100 mL of AcOEt. The organic phase was washed with 50 mL of a 10% Na2S2O3 in water solution and 50 mL of brine, dried over Na₂SO₄, filtered and evaporated to dryness. The resulting crude compound was then purified by flash chromatography (BIOTAGE, redistep 40 g silica gel column) with the following gradient: A: cyclohexane/B: AcOEt: 0% B for 3 min, 0% to 15% B in 20 min, 15% B for 5 min to give the title compound as a brown solid (1.4156 g, 81%). Rf=0.17 (cyclohexane 9/AcOEt 1); 1H-NMR (400 MHz, CDCl₃): δ 7.88 (d, 1H), 7.78-7.85 (m, 1H), 7.12-7.26 (m, 3H), 3.98 (s, 2H); HPLC-MS: 1.72 min, 303.9 [M+H]+;

### Intermediate 77

### (Trans)-8-({[1-(2-fluorophenyl)-4-iodo-1H-pyrazol-3-yl]amino}methyl)-3-(2-pyridinyl)-1-oxa-3-azaspiro[4.5]decan-2-one

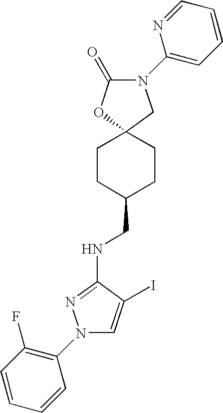

(Trans)-2-oxo-3-(2-pyridinyl)-1-oxa-3-azaspiro[4.5]decane-8-carbaldehyde (this may be prepared as described for intermediate 26, 337 mg, 1.295 mmol) and 1-(2-fluorophenyl)-4-iodo-1H-pyrazol-3-amine (Intermediate 76, 392 mg, 1.295 mmol) were dissolved in 1,2-dichloroethane (4 mL). Then titanium(IV) isopropoxide (0.759 mL, 2.59 mmol) was added and the mixture was stirred at 60° C. for 6 h 35 min. The solution was cooled to r.t. and methanol (2.56 mL) was added followed by sodium borohydride (147 mg, 3.88 mmol). The mixture was stirred at r.t. for 14 h 50 min. 10 mL of a saturated solution of K₂CO₃ were added. The mixture was stirred at r.t. for 5 min, filtered and the cake was washed with 75 mL of AcOEt. The biphasic solution was transferred to a separatory funnel, the organic phase was kept and the aqueous phase was extracted with 25 mL of AcOEt. The combined organic layers were washed once with 25 mL of brine, dried over Na₂SO4, filtered and evaporated to dryness. The resulting residue was then purified by flash chromatography (ISCO COMPANION, 120 g silica gel column) with the following gradient: A: Cyclohexane/B: AcOEt: 0% B for 3.5 min, 0% to 10% B in 10.5 min, 10% B for 9.3 min, 10% to 25% B in 8.2 min, 25% B for 10.5 min to give the title compound as a brown oil (482 mg, 65%). Rf=0.04 (cyclohexane 9/AcOEt 1);

1H-NMR (400 MHz, CDCl₃): δ 8.33-8.36 (m, 1H), 8.26-8.31 (m, 1H), 7.84-7.92 (m, 2H), 7.66-7.77 (m, 1H), 7.13-7.27 (m, 3H), 7.02-7.08 (m, 1H), 4.07 (s, 2H), 3.78 (t, 1H), 3.29 (t, 2H), 1.98-2.10 (m, 3H), 1.62-1.96 (m, 4H), 1.13-1.35 (m, 2H); HPLC-MS: 2.97 min, 548.1 [M+H]+.

### Intermediate 78

### 4-fluoro-1-(2-fluorophenyl)-1H-pyrazol-3-amine

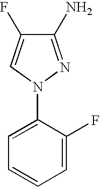

1-(2-fluorophenyl)-1H-pyrazol-3-amine (this may be prepared according to the procedure described in J. Org. Chem. 2005, 70, 922, 300 mg, 1.693 mmol) was dissolved in tetrahydrofuran (6 mL). Then N-fluoro-N-(phenylsulfonyl)benzenesulfonamide (561 mg, 1.778 mmol) was added and the mixture was stirred at 60° C. for 23 h. The mixture was allowed to reach r.t. and 5 mL of MeOH were added. The solution was passed through a 20 g SCX cartridge. The cartridge was washed twice with 50 mL of MeOH and the compound was released with 75 mL of a 2M solution of NH3 in MeOH. Solvents were removed under reduced pressure and the crude compound was purified by flash chromatography (ISCO COMPANION, 12 g silica gel column): A: Cyclohexane/B: AcOEt: 0% B for 1.8 min, 0% to 25% B in 17.9 min, 25% B for 3.6 min to give the title compound as a brown solid (22.3 mg, 6%).

1H-NMR (400 MHz, CDCl3): δ 7.73-7.85 (m, 2H), 7.08-7.25 (m, 3H), 3.85 (brs, 2H); HPLC-MS: 1.94 min, 196.1 [M+H]+;

### Intermediate 79

### (Trans)-8-({[4-fluoro-1-(2-fluorophenyl)-1H-pyrazol-3-yl]amino}methyl)-1-oxa-3-azaspiro[4.5]decan-2-one

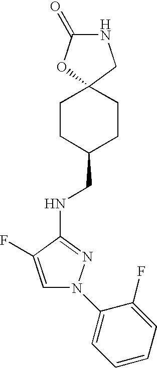

The title compound was made in a similar fashion to the preparation of Intermediate 77 using (trans)-2-oxo-1-oxa-3-azaspiro[4.5]decane-8-carbaldehyde (this may be prepared as described for Intermediate 18, 141 mg, 0.769 mmol) and 4-fluoro-1-(2-fluorophenyl)-1H-pyrazol-3-amine (prepared in a similar fashion to Intermediate 78, 150 mg, 0.769 mmol) to afford the title compound as a white solid (144 mg, 51%).

1H-NMR (400 MHz, CDCl3): δ 7.76-7.87 (m, 2H), 7.09-7.24 (m, 3H), 5.13 (s, 1H), 3.69 (t, 1H), 3.37-3.44 (m, 2H), 3.26 (t, 2H), 1.93-2.06 (m, 4H), 1.71-1.89 (m, 3H), 1.06-1.22 (m, 2H); R_{f}=0.53 (AcOEt); UPLC-MS: 0.69 min, 363.03 [M+H]+, 725.09 [2M+H]+;

### Intermediate 80

### Ethyl (trans)-2-oxo-3-(3-pyridazinyl)-1-oxa-3-azaspiro[4.5]decane-8-carboxylate

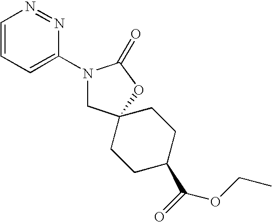

Ethyl (cis)-2-oxo-1-oxa-3-azaspiro[4.5]decane-8-carboxylate (prepared in a similar fashion to Intermediate 16, 10 g, 44.0 mmol), K3PO4 (28.0 g, 132 mmol), copper(I) iodide (0.838 g, 4.40 mmol) and 3-chloropyridazine (6.05 g, 52.8 mmol) were collected into a 250 ml reaction flask, deareated, and then suspended in 1,4-dioxane (150 ml) under nitrogen. Trans-1,2-diaminocyclohexane (1.058 ml, 8.80 mmol) was added to the resulting brown mixture. The reaction was then heated to reflux (ext temp. 130° C., int temp 105° C.). The reaction mixture was stirred at that temperature for ˜24 h, then quenched.

The reaction mixture was taken up with DCM (1000 ml) and poured into water (300 ml) containing 10 ml of ammonium hydroxide and left to stir for 10 min. Then, the resulting organic phase was washed with water (2×100 ml) and brine (2×100 ml), dried over Na2SO4, filtered and then concentrated. The resulting crude was then purified twice with Biotage SP1, with a 65i Silica column, using cyclohexane/EtOAc as eluent to afford the title compound (1.6 g). 1H NMR (400 MHz, CDCl3) δ 8.96 (dd, 1H), 8.56 (dd, 1H), 7.50 (dd, 1H), 4.20 (s, 2H), 4.18 (q, 2H), 2.51 (sept, 1H), 2.07-2.18 (m, 2H), 1.97-2.06 (m, 2H), 1.87-1.96 (m, 2H), 1.75-1.86 (m, 2H), 1.29 (t, 3H); UPLC-MS: 0.60 min, 306 [M+H]+.

The epimer ethyl (cis)-2-oxo-3-(3-pyridazinyl)-1-oxa-3-azaspiro[4.5]decane-8-carboxylate was also isolated (5.0 g). 1H NMR (400 MHz, CDCl3) δ 8.96 (dd, 1H), 8.57 (dd, 1H), 7.50 (dd, 1H), 4.18 (q, 2H), 4.14 (s, 2H), 2.41 (sept, 1H), 2.12-2.21 (m, 2H), 1.95-2.10 (m, 4H), 1.65-1.76 (m, 2H), 1.29 (t, 3H).

### Intermediate 81

### (Trans)-8-(hydroxymethyl)-3-(3-pyridazinyl)-1-oxa-3-azaspiro[4.5]decan-2-one

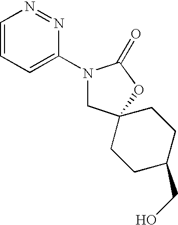

The title compound was made in a similar fashion to the preparation of Intermediate 22 using ethyl (trans)-2-oxo-3-(3-pyridazinyl)-1-oxa-3-azaspiro[4.5]decane-8-carboxylate (Intermediate 80, 1.6 g, 5.24 mmol) to give the title compound (1.1 g).

1H NMR (400 MHz, CDCl3) δ 8.97 (dd, 1H), 8.56 (dd, 1H), 7.50 (dd, 1H), 4.21 (s, 2H), 3.55 (d, 2H), 1.83-2.08 (m, 6H), 1.57-1.72 (m, 1H), 1.15-1.29 (m, 2H); UPLC-MS: 0.44 min, 264 [M+H]+.

### Intermediate 82

### (Trans)-2-oxo-3-(3-pyridazinyl)-1-oxa-3-azaspiro[4.5]decane-8-carbaldehyde

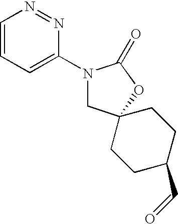

The title compound was made in a similar fashion to the preparation of Intermediate 23 using (trans)-8-(hydroxymethyl)-3-(3-pyridazinyl)-1-oxa-3-azaspiro[4.5]decan-2-one Intermediate 81, 500 mg, 1.90 mmol) to give the title compound (490 mg).

1H NMR (400 MHz, CDCl3) δ 9.73 (s, 1H), 8.95 (dd, 1H), 8.54 (dd, 1H), 7.49 (dd, 1H), 4.13 (s, 2H), 2.44-2.53 (m, 1H), 2.10-2.20 (m, 2H), 1.77-2.02 (m, 6H); UPLC-MS: 0.49 min, 262 [M+H]+.

### Intermediate 83

### (3E)-4-amino-4-(ethyloxy)-1,1,1-trifluoro-3-buten-2-one

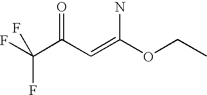

A 2.0M solution of ammonia in methanol (1.178 ml, 2.357 mmol) was added to a solution of 4,4-bis(ethyloxy)-1,1,1-trifluoro-3-buten-2-one (prepared as described in Synthesis 1986, 1013-1014, 500 mg, 2.357 mmol) in dry acetonitrile (9.5 ml) at room temperature and the mixture was stirred for 2 h under nitrogen atmosphere. Solvents were evaporated in vacuo and the residue was dissolved in DCM, washed with water which was then back-extracted with DCM. The combined organic extracts were dried (Na₂SO₄), filtered and concentrated in vacuo to give the title compound (406 mg, 94%) which was used without further purification.

1H NMR (400 MHz, CDCl3) δ 9.52-10.05 (m, 1H), 5.45-5.87 (m, 1H), 5.14 (s, 1H), 4.16 (q, 2H), 1.42 (t, 3H); UPLC-MS: 0.59 min, 184 [M+H]+.

### Intermediate 84

### 1-(2-fluorophenyl)-5-(trifluoromethyl)-1H-pyrazol-3-amine

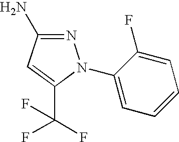

(2-fluorophenyl)hydrazine hydrochloride (397 mg, 2.439 mmol) and triethylamine (0.340 ml, 2.439 mmol) were added to a stirred solution of (3E)-4-amino-4-(ethyloxy)-1,1,1-trifluoro-3-buten-2-one (Intermediate 83, 406 mg, 2.217 mmol) in ethanol (15 ml) at room temperature. The mixture was stirred at 96° C. under a nitrogen atmosphere for 9 h then left at room temperature overnight. Additional (2-fluorophenyl)hydrazine hydrochloride (357 mg, 2.2 mmol) was treated with triethylamine (0.340 ml, 2.44 mmol) in ethanol (1 ml) for 10 min (until complete dissolution) and the resulting solution was added to the reaction mixture which was refluxed for 1 h (external temperature 100-110° C.). Solvent was removed in vacuo and the residue was dissolved in DCM, washed with water which was then back-extracted with DCM (2×10 ml). The combined organic extracts were dried (Na₂SO₄), filtered and concentrated in vacuo to give a residue which was purified by silica gel chromatography (biotage 25M+ column) with DCM 100% eluent affording the title compound as pale orange solid (97.6 mg, 16%).

1H NMR (400 MHz, CDCl3) δ 3.79-3.96 (brs, 2H) 5.92 (s, 1H) 7.26-7.31 (m, 1H) 7.31-7.37 (m, 1H) 7.45-7.54 (m, 1H) 7.53-7.62 (m, 1H); UPLC-MS: 0.64 min, 246 [M+H]+.

### EXAMPLES

### Example 1

### Preparation of Compounds of Formula (IIa)

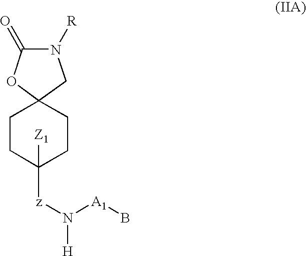

### Example 1-1

### (Cis)3-Phenyl-8-({[4-(2-pyridinyl)-1,3-thiazol-2-yl]amino}methyl)-1-oxa-3-azaspiro[4.5]-decan-2-one

A mixture of 2-oxo-3-phenyl-1-oxa-3-azaspiro[4.5]decane-8-carbaldehyde (Intermediate 5, 0.277 mmol) and 4-(2-pyridinyl)-1,3-thiazol-2-amine (9.9 mg, 0.056 mmol, Fluorochem) in anhydrous DCM (1.5 ml) was stirred for 10 min, then acetic acid (15.86 μl, 0.28 mmol) and polystyrene-supported cyanoborohydride (125 mg, 2.5-4.5 mml/g loading, ˜2.25 eq.) were added. The reaction was heated under microwave irradiation to 110° C. for two 7 minutes cycles. The resin was filtered off washing with DCM. The filtrate was partitioned between saturated aqueous sodium hydrogencarbonate solution and DCM; the organic layer was filtered through a hydrophobic membrane and concentrated under vacuum. The crude (80 mg) was purified by MDAP to give the title compound (11.9 mg, 11%, mixture 85:15 of two isomers, cis:trans).

### Example 1-2

### (Trans)-3-Phenyl-8-({[4-(2-pyridinyl)-1,3-thiazol-2-yl]amino}methyl)-1-oxa-3-azaspiro-[4.5]decan-2-one

A solution of chlorotriisopropoxytitanium (130 mg, 0.50 mmol) in dichloromethane (1 ml) was added to a stirred mixture of (trans)-2-oxo-3-phenyl-1-oxa-3-azaspiro[4.5]decane-8-carbaldehyde (intermediate 7, 59 mg, 0.23 mmol) and 4-(2-pyridinyl)-1,3-thiazol-2-amine (44 mg, 0.25 mmol) in dichloromethane (3 ml) at 0° C. The resulting solution was allowed to warm to room temperature and was stirred for 18 hours, then sodium triacetoxyborohydride (244 mg, 1.15 mmol) and 2 drops of glacial acetic acid were added. The mixture was stirred for 2 hours at room temperature then quenched with a saturated aqueous solution of sodium hydrogencarbonate (4 ml) and diluted with dichloromethane (10 ml). Sufficient aqueous sodium hydroxide solution was added to avoid emulsioning and the mixture was filtered through a hydrophobic frit (PhaseSep cartridge) washing twice with more dichloromethane. The combined organic phases were concentrated under reduced pressure and the residue was triturated with dichloromethane to leave a white solid. The supernatant liquid was loaded onto a NH column (12M, Biotage) and eluted with a gradient of 25-80% EtOAc/cyclohexane. The fractions from this chromatography that were enriched in the title compound were combined with the solid isolated by trituration and the mixture concentrated under reduced pressure. The residue was chromatographed on SiO₂ eluting with a gradient of 5-10% MeOH/dichloromethane to give a mixture of the title compound and (trans)-8-(hydroxymethyl)-3-phenyl-1-oxa-3-azaspiro[4.5]decan-2-one. This mixture was purified on an SCX cartridge eluting first with MeOH/dichloromethane, to elute (trans)-8-(hydroxymethyl)-3-phenyl-1-oxa-3-azaspiro[4.5]decan-2-one, and then with 2M NH₃ in MeOH/dichloromethane to give the title compound as a white solid (63 mg).

### Example 1-3

### (Trans)-8-({[4-(6-Methyl-2-pyridinyl)-1,3-thiazol-2-yl]amino}methyl)-3-phenyl-1-oxa-3-azaspiro[4.5]decan-2-one hydrochloride

(Trans)-8-({[4-(6-Methyl-2-pyridinyl)-1,3-thiazol-2-yl]amino}methyl)-3-phenyl-1-oxa-3-aza-spiro[4.5]decan-2-one (Example 1-4, free base, 0.038 g, 0.087 mmol) was suspended in dry diethyl ether (1.5 ml) and HCl 1M in diethyl ether (0.105 mL, 0.105 mmol) was added. A precipitate was formed that was triturated with diethyl ether then dried under vacuum to give the title compound as yellow solid (37.9 mg, 92.7%).

### Example 1-4

### (Trans)-8-({[4-(6-Methyl-2-pyridinyl)-1,3-thiazol-2-yl]amino}methyl)-3-phenyl-1-oxa-3-azaspiro[4.5]decan-2-one

(Trans)-2-Oxo-3-phenyl-1-oxa-3-azaspiro[4.5]decane-8-carbaldehyde (prepared in a similar fashion to Intermediate 7, 0.03 g, 0.116 mmol) and 4-(6-methyl-2-pyridinyl)-1,3-thiazol-2-amine (for a preparation see Journal of Medicinal & Pharmaceutical Chemistry, 1961, 3, 561-6; 0.024 g, 0.127 mmol) in dry DCM (2 ml) were stirred at room temperature for 0.5 hour. The mixture was cooled to 0° C. and a solution of titanium chloride triisopropoxide (0.055 mL, 0.232 mmol) in DCM (0.5 mL) was added. The mixture was allowed to slowly warm to room temperature and stirred overnight. 3 drops of glacial acetic acid and sodium triacetoxyborohydride (0.123 g, 0.58 mmol) were added and the mixture was stirred at room temperature for 2 hours. The mixture was diluted with DCM. NaOH 30% aqueous solution was added and the mixture was extracted with DCM (3×15 mL); each extract was passed through a phase-separation syringe filter. The organics were combined and concentrated under vacuum to give a residue. The residue was purified by MDAP. Product fractions were combined, concentrated under vacuum, basified with saturated aqueous NaHCO3 solution, extracted with DCM, which was then passed through a phase-separation syringe filter. The organics were combined and concentrated under vacuum to give the title compound as yellow foam (39.2 mg, 78%).

### Example 1-5

### (Trans)-8-({[4-(3-Methyl-2-pyridinyl)-1,3-thiazol-2-yl]amino}methyl)-3-phenyl-1-oxa-3-azaspiro[4.5]decan-2-one hydrochloride

The title compound was made in a similar fashion to the preparation of Example 1-3 using (trans)-8-({[4-(3-methyl-2-pyridinyl)-1,3-thiazol-2-yl]amino}methyl)-3-phenyl-1-oxa-3-aza-spiro[4.5]decan-2-one (Example 1-6, free base) to give the title compound as yellowish solid (10.6 mg, 90%).

### Example 1-6

### (Trans)-8-({[4-(3-Methyl-2-pyridinyl)-1,3-thiazol-2-yl]amino}methyl)-3-phenyl-1-oxa-3-azaspiro[4.5]decan-2-one

The title compound was made in a similar fashion to the preparation of Example 1-4 replacing 4-(6-methyl-2-pyridinyl)-1,3-thiazol-2-amine with 4-(3-methyl-2-pyridinyl)-1,3-thiazol-2-amine (Intermediate 13) to give the title compound as a brownish foam (10.9 mg, 21.6%).

### Example 1-7

### (Trans)-3-(2-pyridinyl)-8-({[4-(2-pyridinyl)-1,3-thiazol-2-yl]amino}methyl)-1-oxa-3-azaspiro[4.5]decan-2-one dihydrochloride

Chlorotitanium triisopropoxide (0.27 ml, 1.13 mmol) in 5 ml of DCM was added to a stirred mixture of 4-(2-pyridinyl)-1,3-thiazol-2-amine (100 mg, 0.565 mmol) and (trans)-2-oxo-3-(2-pyridinyl)-1-oxa-3-azaspiro[4.5]decane-8-carbaldehyde (Intermediate 26, 133.7 mg, 0.514 mmol) in 15 ml of DCM. The mixture became yellow and was left stirring under N₂ at rt for 48 h. Then sodium triacetoxyborohydride (544 mg, 2.57 mmol) and (0.029 ml, 0.514 mmol) of acetic acid were added. The crude was poured into a saturated solution of NaHCO₃ (20 ml) and extracted with DCM (50 ml), it gave an emulsion so it was added NaOH 2M (3 ml) and the solution was filtered using a phase separator tube and the organic phase was concentrated under vacuo. The crude was purified using a Biotage 25M NH column eluting in gradient with DCM:Et₂O from 100:0 to 70:30 to give (120 mg, 55% yield) of trans-3-(2-pyridinyl)-8-({[4-(2-pyridinyl)-1,3-thiazol-2-yl]amino}methyl)-1-oxa-3-azaspiro[4.5]decan-2-one.

^{1H} NMR (400 MHz, CDCl₃): δ 8.62-8.53 (1H, m), 8.33-8.29 (1H, m), 8.24 (1H, dt), 7.91 (1H, d), 7.73-7.66 (2H, m), 7.19-7.15 (1H, m), 7.04-7.00 (1H, m), 5.83-5.75 (1H, m), 5.30-5.27 (1H, m), 3.98-4.03 (2H, m), 3.22 (2H, t), 2.01-1.90 (4H, m), 1.84-1.67 (3H, m), 1.28-1.12 (2H, m).

To a solution of trans-3-(2-pyridinyl)-8-({[4-(2-pyridinyl)-1,3-thiazol-2-yl]amino}methyl)-1-oxa-3-azaspiro[4.5]decan-2-one (120 mg, 0.285 mmol) in DCM (3 ml) was added drop by drop under stirring a solution 1M in Et₂O of HCl (0.626 ml, 0.626 mmol). The solution was left at rt under stirring for 30 minutes and then the precipitate was separated, triturated with Et₂O, concentrated under a flow of nitrogen and dried for 18 h under high vacuum at 40° C. to afford the title compound (132 mg, 89% yield).

### Example 1-8

### (Trans)-3-(4-fluorophenyl)-8-({[4-(2-pyridinyl)-1,3-thiazol-2-yl]amino}methyl)-1-oxa-3-azaspiro[4.5]decan-2-one

The title compound was made in a similar fashion to the preparation of Example 1-7 replacing (trans)-2-oxo-3-(2-pyridinyl)-1-oxa-3-azaspiro[4.5]decane-8-carbaldehyde with trans-3-(4-fluorophenyl)-2-oxo-1-oxa-3-azaspiro[4.5]decane-8-carbaldehyde (Intermediate 29, 35.8 mg, 0.129 mmol) to give the title compound as a brownish foam (10.9 mg, 21.6%).

### Example 1-9

### (Trans)-3-(2-fluorophenyl)-8-({[4-(2-pyridinyl)-1,3-thiazol-2-yl]amino}methyl)-1-oxa-3-azaspiro[4.5]decan-2-one hydrochloride

The title compound was made in a similar fashion to the preparation of Example 1-7 replacing (trans)-2-oxo-3-(2-pyridinyl)-1-oxa-3-azaspiro[4.5]decane-8-carbaldehyde with trans-3-(2-fluorophenyl)-2-oxo-1-oxa-3-azaspiro[4.5]decane-8-carbaldehyde (Intermediate 32, 70 mg, 0.252 mmol) and purifying by MDAP to give the title compound (15 mg, 12%).

### Example 1-10

### (Cis)-3-(3-pyridinyl)-8-({[4-(2-pyridinyl)-1,3-thiazol-2-yl]amino}methyl)-1-oxa-3-azaspiro-[4.5]decan-2-one hydrochloride

(Cis)-2-oxo-3-(3-pyridinyl)-1-oxa-3-azaspiro[4.5]decane-8-carbaldehyde (Intermediate 41, 55 mg, 0.211 mmol) and 4-(2-pyridinyl)-1,3-thiazol-2-amine (37.4 mg, 0.211 mmol) were dissolved in dichloromethane (4 mL) and cooled to 0° C. Then chlorotitanium triisopropoxide (0.101 mL, 0.423 mmol) was added and the mixture was allowed to warm up and stirred at r.t. overnight. Then sodium triacetoxyborohydride (224 mg, 1.057 mmol) and acetic acid (0.121 mL, 2.113 mmol) were added and the mixture was stirred at r.t for 4 h. The mixture was then taken up with DCM (20 ml) and extracted with NaHCO₃. The crude was then purified with Biotage SP1, on a 12M NH, using a mixture of cyclohexane/EtOAc as eluent. (Cis)-3-(3-pyridinyl)-8-({[4-(2-pyridinyl)-1,3-thiazol-2-yl]amino}methyl)-1-oxa-3-azaspiro[4.5]decan-2-one (57 mg), was eluted with ca 65% EtOAc.

¹H NMR (400 MHz, CDCl₃): 8.61 (dq, 1H), 8.57 (d, 1H), 8.41 (d, 1H), 8.26 (dq, 1H), 7.94 (dt, 1H), 7.74 (dt, 1H), 7.37-7.32 (m, 1H), 7.32 (s, 1H), 7.22-7.18 (m, 1H), 5.31 (br s, 1H), 3.78 (s, 2H), 3.33 (t, 2H), 2.23-1.25 (m, 9H). UPLC-MS: 0.48 min, 422 [M+H]+.

This was dissolved in DCM and treated with 1.1 eq of 1M HCl in diethylether to give the title compound (67 mg).

### Example 1-11

### (Trans)-3-(3-pyridinyl)-8-({[4-(2-pyridinyl)-1,3-thiazol-2-yl]amino}methyl)-1-oxa-3-azaspiro[4.5]decan-2-one dihydrochloride

The title compound was made in a similar fashion to the preparation of Example 1-10 replacing (cis)-2-oxo-3-(3-pyridinyl)-1-oxa-3-azaspiro[4.5]decane-8-carbaldehyde with (trans)-2-oxo-3-(3-pyridinyl)-1-oxa-3-azaspiro[4.5]decane-8-carbaldehyde (Intermediate 44, 50 mg, 0.192 mmol) to give the title compound (40 mg) as a yellow solid.

### Example 1-12

### (Trans)-8-({[4-(3-fluoro-2-pyridinyl)-1,3-thiazol-2-yl]amino}methyl)-3-(3-pyridinyl)-1-oxa-3-azaspiro[4.5]decan-2-one dihydrochloride

The title compound was made in a similar fashion to the preparation of Example 1-11 replacing 4-(2-pyridinyl)-1,3-thiazol-2-amine with 4-(3-fluoro-2-pyridinyl)-1,3-thiazol-2-amine (Intermediate 47, 52.5 mg, 0.269 mmol) to give the title compound (35 mg).

### Example 1-13

### (Trans)-3-(2-methyl-3-pyridinyl)-8-({[4-(2-pyridinyl)-1,3-thiazol-2-yl]amino}methyl)-1-oxa-3-azaspiro[4.5]decan-2-one dihydrochloride

(Trans)-3-(2-methyl-3-pyridinyl)-2-oxo-1-oxa-3-azaspiro[4.5]decane-8-carbaldehyde (Intermediate 50, 50 mg, 0.182 mmol) 4-(2-pyridinyl)-1,3-thiazol-2-amine (32.3 mg, 0.182 mmol) and chlorotitanium triisopropoxide (0.087 ml, 0.365 mmol) were collected in dichloromethane (2 ml) and stirred at rt overnight. Then, sodium triacetoxyborohydride (193 mg, 0.911 mmol) and acetic acid (0.104 ml, 1.823 mmol) were added and the resulting mixture was stirred at rt for further 4 h. Then, the reaction mixture was taken up with DCM (20 ml) and treated with sat NaHCO₃ (2 ml). It was then filtered over a separation tube and concentrated to afford a crude oil (100 mg). This was purified with Biotage SP1, over a 12+M KP—NH cartridge, using cyclohexane and ethyl acetate as eluent to give (trans)-3-(2-methyl-3-pyridinyl)-8-({[4-(2-pyridinyl)-1,3-thiazol-2-yl]amino}methyl)-1-oxa-3-azaspiro[4.5]decan-2-one as colourless solid (60 mg).

¹H NMR (400 MHz, CDCl₃): 8.60 (dq, 1H), 8.50 (dd, 1H), 7.91 (dt, 1H), 7.73 (dt, 1H), 7.60 (dd, 1H), 7.30 (s, 1H), 7.25-7.17 (m, 2H), 3.73 (s, 2H), 3.30 (m, 2H), 2.57 (s, 3H), 2.16-1.13 (m, 9H).

This was dissolved in DCM (2 ml) and reacted with 2.1 equiv of 1M HCl in Et₂O to afford the title compound as yellow solid (60 mg).

### Example 1-14

### (Trans)-8-({[4-(2-pyridinyl)-1,3-thiazol-2-yl]amino}methyl)-3-(5-pyrimidinyl)-1-oxa-3-azaspiro[4.5]decan-2-one dihydrochloride

The title compound was made in a similar fashion to the preparation of Example 1-13 replacing (trans)-3-(2-methyl-3-pyridinyl)-2-oxo-1-oxa-3-azaspiro[4.5]decane-8-carbaldehyde with (trans)-2-oxo-3-(5-pyrimidinyl)-1-oxa-3-azaspiro[4.5]decane-8-carbaldehyde (Intermediate 53, 20 mg, 0.077 mmol) to give the title compound as a yellow solid (13 mg).

### Example 1-15

### (Cis)-8-methyl-3-(2-pyridinyl)-8-({[4-(2-pyridinyl)-1,3-thiazol-2-yl]amino}methyl)-1-oxa-3-azaspiro[4.5]decan-2-one dihydrochloride

Chlorotitanium triisopropoxide (157 mg, 0.60 mmol) in 1 ml of dichloromethane was added to a stirred mixture of 4-(2-pyridinyl)-1,3-thiazol-2-amine (35.5 mg, 0.20 mmol) and (cis)-8-methyl-2-oxo-3-(2-pyridinyl)-1-oxa-3-azaspiro[4.5]decane-8-carbaldehyde (Intermediate 58, 55 mg, 0.20 mmol) in 3 ml of dichloromethane. The mixture became yellow and was left stirring under N2 at rt for 48 hours. Sodium triacetoxyborohydride (212 mg, 1.002 mmol) and acetic acid (0.011 mL, 0.200 mmol) were added and the reaction mixture was allowed to stir for 8 hours. Add another quantity of acetic acid (0.011 mL, 0.200 mmol) and stir for 18 hours. Add a third quantity of acetic acid (0.011 mL, 0.200 mmol) and stir for another 8 hours. Add more sodium triacetoxyborohydride (106 mg, 0.501 mmol) and more acetic acid (0.011 mL, 0.200 mmol) and allow to stir for 18 hours. Quench with saturated potassium carbonate solution (10 ml), dilute with dichloromethane (20 ml) and filter through a hydrophobic frit (Phase Separator cartridge) washing with dichloromethane (2×10 ml). Evaporate under reduced pressure and chromatograph the residue by Biotage (0-25% diethyl ether/dichloromethane; 25M column) to give (cis)-8-methyl-3-(2-pyridinyl)-8-({[4-(2-pyridinyl)-1,3-thiazol-2-yl]amino}methyl)-1-oxa-3-azaspiro[4.5]decan-2-one (45 mg) as a colourless glass.

1H-NMR (400 MHz, acetone-d6): δ 8.53 (1H, d), 8.33 (1H, d), 8.19 (1H, d), 8.00 (1H, d), 7.77-7.83 (2H, m), 7.29 (1H, s), 7.22 (1H, dd), 7.08 (1H, dd), 6.90 (1H, t), 4.00 (2H, s), 3.48 (2H, d), 2.00-2.09 (2H, m), 1.94 (2H, td), 1.84 (2H, td), 1.51 (2H, dt), 1.13 (3H, s); UPLC-MS: 0.62 min, 436 [M+H]+, 218 [M+2H]2+

1.0M HCl in ether (0.296 ml, 0.296 mmol) was added to a stirred solution of (cis)-8-methyl-3-(2-pyridinyl)-8-({[4-(2-pyridinyl)-1,3-thiazol-2-yl]amino}methyl)-1-oxa-3-azaspiro[4.5]decan-2-one (43 mg, 0.099 mmol) in dichloromethane. A white precipitate forms immediately. Add sufficient methanol to make a homogeneous solution. Stir for 30 minutes during which a white solid precipitates from the yellow solution. Filter the mixture washing the filter cake with diethyl ether (2×5 ml) then dry under vacuum at 60° C. for 4 hours to give 50 mg of a yellow solid. Dissolve in MeOH (1 ml) and evaporate three times then dry under vacuum at 60° C. for 6 hours to give the title compound (49 mg) as a yellow solid.

### Example 1-16

### (Trans)-8-methyl-3-(2-pyridinyl)-8-({[4-(2-pyridinyl)-1,3-thiazol-2-yl]amino}methyl)-1-oxa-3-azaspiro[4.5]decan-2-one dihydrochloride

Chlorotitanium triisopropoxide (0.084 mL, 0.350 mmol) in dichloromethane (0.5 ml) was added to a stirred mixture of (trans)-8-methyl-2-oxo-3-(2-pyridinyl)-1-oxa-3-azaspiro[4.5]decane-8-carbaldehyde (Intermediate 59, 32 mg, 0.117 mmol) and 4-(2-pyridinyl)-1,3-thiazol-2-amine (20.67 mg, 0.117 mmol) in dichloromethane (2 ml) at room temperature in a glass vial. The resulting yellow mixture was allowed to stir overnight (˜18 hours). Add more chlorotitanium tri-isopropoxide (0.041 mL, 0.175 mmol) and allow to stir for another 24 hours. Add sodium triacetoxyborohydride (124 mg, 0.583 mmol) and acetic acid (0.020 mL, 0.350 mmol) and allow to stir for 6 hours. Add further acetic acid (0.020 mL, 0.350 mmol) and stir overnight (˜18 hours). Dilute with dichloromethane (5 ml) then quench with saturated K₂CO₃ solution (3 ml). Add sufficient water such that the aqueous phase moves above the organic phase then filter through a hydrophobic frit (Phase Separator cartridge), washing with further dichloromethane (3×5 ml). Evaporate the organic phase and chromatograph the residue twice via Biotage (first purification 100% CH2Cl2; 12M NH column; second purification 20-50% EtOAc/cyclohexane; 12M NH2 column) to give (trans)-8-methyl-3-(2-pyridinyl)-8-({[4-(2-pyridinyl)-1,3-thiazol-2-yl]amino}methyl)-1-oxa-3-azaspiro[4.5]decan-2-one (24 mg) as a white foam.

1H-NMR (400 MHz, CDCl₃): δ 8.61 (1H, d), 8.33-8.36 (1H, m), 8.27 (1H, d), 7.92 (1H, d), 7.69-7.78 (2H, m), 7.30 (1H, s), 7.17-7.22 (1H, m), 7.05 (1H, dd), 5.24 (1H, brt), 4.04 (2H, s), 3.30 (2H, d), 1.97-2.08 (2H, m), 1.83-1.94 (2H, m), 1.65-1.80 (2H, m), 1.52-1.64 (2H, m), 1.12 (3H, s); UPLC-MS: 0.63 min, 436 [M+H]+, 218 [M+2H]2+

1.0M HCl in diethyl ether (0.152 ml, 0.152 mmol) was added to a stirred solution of (trans)-8-methyl-3-(2-pyridinyl)-8-({[4-(2-pyridinyl)-1,3-thiazol-2-yl]amino}methyl)-1-oxa-3-azaspiro[4.5]decan-2-one (22 mg, 0.051 mmol) in dichloromethane (2 ml) and methanol (0.1 ml) at room temperature in a glass vial. Stir for 1 hour then evaporate volatiles under reduced pressure. Triturate the residue with diethyl ether (4 ml), filter washing the filter cake with more ether (2×2 ml). The yellow solid was collected and dried at 60° C. under reduced pressure for 3 hours to give the title compound (21 mg) as a yellow solid.

### Example 1-17

### (Trans)-3-(6-methyl-2-pyridinyl)-8-({[4-(2-pyridinyl)-1,3-thiazol-2-yl]amino}methyl)-1-oxa-3-azaspiro[4.5]decan-2-one hydrochloride

The title compound was made in a similar fashion to the preparation of Example 1-13 replacing (trans)-3-(2-methyl-3-pyridinyl)-2-oxo-1-oxa-3-azaspiro[4.5]decane-8-carbaldehyde with (trans)-3-(6-methyl-2-pyridinyl)-2-oxo-1-oxa-3-azaspiro[4.5]decane-8-carbaldehyde (Intermediate 62, 24 mg, 0.087 mmol) to give the title compound as a yellow solid (6 mg).

### Example 1-18

### (Trans)-3-(6-fluoro-2-pyridinyl)-8-({[4-(2-pyridinyl)-1,3-thiazol-2-yl]amino}methyl)-1-oxa-3-azaspiro[4.5]decan-2-one

The title compound was made in a similar fashion to the preparation of Example 1-13 replacing (trans)-3-(2-methyl-3-pyridinyl)-2-oxo-1-oxa-3-azaspiro[4.5]decane-8-carbaldehyde with (trans)-3-(6-fluoro-2-pyridinyl)-2-oxo-1-oxa-3-azaspiro[4.5]decane-8-carbaldehyde (Intermediate 64, 21 mg, 0.075 mmol) to give the title compound (15.4 mg; 29%).

### Example 1-19

### (Trans)-3-(2-pyridinyl)-8-(1-{[4-(2-pyridinyl)-1,3-thiazol-2-yl]amino}ethyl)-1-oxa-3-azaspiro[4.5]decan-2-one dihydrochloride

To stirred methylmagnesium bromide (3M in ether) (0.309 ml, 0.928 mmol) in THF (5 ml) at −78° C. under nitrogen was added dropwise a solution of (trans)-8-(1H-1,2,3-benzotriazol-1-yl{[4-(2-pyridinyl)-1,3-thiazol-2-yl]amino}methyl)-3-(2-pyridinyl)-1-oxa-3-azaspiro[4.5]decan-2-one (Intermediate 65, 200 mg, 0.371 mmol) in tetrahydrofuran (10 ml). The mixture was stirred 30 minutes. The mixture was warmed to room temperature over 45 minutes and stirred 2 hours. The mixture was poured into water (20 ml) and extracted twice with ethyl acetate (15 ml). The combined organic extracts were washed with water, filtered through a hydrophobic frit (Phase-Sep membrane) and concentrated under vacuum. The crude was purified first on a NH column eluting with dichloromethane/ether (1:0 to 10:1 gradient) and then on a silica column eluting with dichloromethane/methanol/triethylamine (1:0:0 to 95:5+1 drop/50 ml triethylamine). The combined product-containing fractions were converted to the HCl salt. This solid was dissolved in methanol and purified by SCX ion-exchange chromatography eluting with i) methanol, ii) 2M ammonia methanol. The basic fractions were concentrated under vacuum to give (trans)-3-(2-pyridinyl)-8-(1-{[4-(2-pyridinyl)-1,3-thiazol-2-yl]amino}ethyl)-1-oxa-3-azaspiro[4.5]decan-2-one (19.6 mg) as a clear viscous oil.

1H-NMR (400 MHz, CDCl₃): δ 8.60 (1H, d), 8.34 (1H, d), 8.27 (1H, d), 7.92 (1H, d), 7.68-7.76 (2H, m), 7.29 (1H, d), 7.19 (1H, dd), 7.04 (1H, dd), 5.14 (1H, d), 4.05 (2H, s), 3.50-3.60 (1H, m), 1.75-2.10 (6H, m), 1.55-1.67 (1H, m), 1.23-1.42 (2H, m), 1.29 (3H, s).

This was dissolved in dichloromethane (1 ml) and HCl (1M in ether) (0.113 ml, 0.113 mmol) was added. The mixture was left to stand for 10 minutes then concentrated under a flow of nitrogen at 40° C. The residue was dried under vacuum at 40° C. to give the title compound (20.0 mg) as a yellow powder.

### Example 1-20

### (Trans)-8-({[5-fluoro-4-(2-pyridinyl)-1,3-thiazol-2-yl]amino}methyl)-3-(2-pyridinyl)-1-oxa-3-azaspiro[4.5]decan-2-one

The title compound was made in a similar fashion to the preparation of Example 1-13 replacing (trans)-3-(2-methyl-3-pyridinyl)-2-oxo-1-oxa-3-azaspiro[4.5]decane-8-carbaldehyde with (trans)-2-oxo-3-(2-pyridinyl)-1-oxa-3-azaspiro[4.5]decane-8-carbaldehyde (this may be prepared as described for Intermediate 26, 133 mg, 0.512 mmol) and 4-(2-pyridinyl)-1,3-thiazol-2-amine with 5-fluoro-4-(2-pyridinyl)-1,3-thiazol-2-amine (Intermediate 66, 100 mg, 0.512 mmol) to afford the title compound (31 mg), as slightly yellow solid.

All the analytical data are set forth in the following Table 1-1 and in which R, Z, Z₁, A₁ and B are:

| | |
|---|---|
| | (IIA) |
| 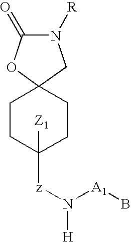 | |

| Cpd. | | | | | |
|---|---|---|---|---|---|
| No. | R | Z | Z₁ | A1—B | Analytical Data |
| 1-1 | Ph | CH₂ | H | 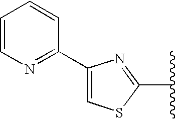 | ¹H NMR (500 MHz, CDCl₃): δ 8.59(1 H, d), 7.94-7.89 (1 H, m), 7.78-7.701 H, m), 7.58-7.53 (2 H, m), 7.42-7.36(3 H, m), 7.29 (1 H, s), 7.20-7.12 (2 H,m), 5.30 (1 H, s), 3.82-3.73 (2 H, m),3.53-3.44 (1 H, m), 3.32-3.24 (2 H, m),2.23-2.00 (3 H, m), 1.92-1.76 (4 H, m);MS, m/z 421 [M + H]⁺ |
| 1-2 | Ph | CH₂ | H | 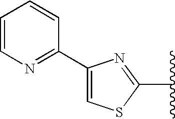 | ¹H NMR (400 MHz, CDCl₃): δ 8.60(1 H, d), 7.92 (1 H, d), 7.74 (1 H, dt),7.58 (2 H, d), 7.40 (2 H, t), 7.31 (1 H,s), 7.20 (1 H, ddd), 7.15 (1 H, t), 5.23(1 H, t), 3.82 (2 H, s), 3.31 (2 H, t), 2.04(4 H, m), 1.90 (3 H, m), 1.25 (2 H, m);m/z (ES): 421 [M + H]⁺. |
| 1-3 | Ph | CH₂ | H | 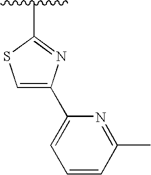 | 1H NMR (500 MHz, DMSO-d₆): δ8.20 (2 H, br s), 8.00 (1 H, br s), 7.78(1 H, br s), 7.58 (2 H, d), 7.51 (1 H, brs), 7.38 (2 H, dd), 7.10 (1 H, dd), 3.83-3.98(2 H, m), 3.17-3.32 (2 H, m), 2.64(3 H, s), 1.90-2.02 (2 H, m), 1.80-1.90(2 H, m), 1.59-1.77 (3 H, m), 1.17-1.31(H, m);HPLC-MS 2: 2.08 min, m/z 435[M + H]+ |
| 1-4 | Ph | CH₂ | H | 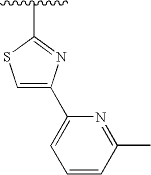 | ¹H NMR (400 MHz, CDCl₃):δ 7.55-7.73 (4 H, m), 7.36-7.44 (2 H,m), 7.30-7.33 (1 H, m), 7.05-7.19 (2 H,m), 3.83 (2 H, s), 3.27-3.34 (2 H, t),2.62 (s, 3 H), 1.98-2.12 (4 H, m),1.79-1.97 (3 H, m), 1.21-1.34 (3 H, m);HPLC-MS 1: 1.84 min, m/z 435[M + H]+ |
| 1-5 | Ph | CH₂ | H | 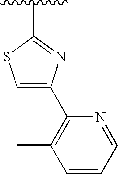 | ^{1H NMR} (500 MHz, DMSO-d₆): δ 8.61-8.70(1 H, m), 8.25-8.44 (2 H, br. s),7.68-7.81 (1 H, br s), 7.52-7.65 (3 H,m), 7.32-7.43 (2 H, m), 7.06-7.18(1 H, m), 3.91 (2 H, br s), 3.32-3.40(2 H, m), 2.64 (3 H, s), 1.58-2.04 (7 H,m), 1.39-1.60 (2 H, m);HPLC-MS 2: 2.09 min, m/z 435,[M + H]+ |
| 1-6 | Ph | CH₂ | H | 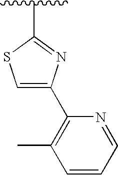 | ¹H NMR (400 MHz, CDCl₃): δ 8.48-8.56(1 H, m), 7.52-7.65 (3 H, m), 7.34-7.46(2 H, m), 7.11-7.23 (2 H, m), 6.91(1 H, s), 3.83 (2 H, s), 3.27-3.35 (2 H,t), 2.57 (3 H, s), 1.78-2.11 (4 H, m),1.20-1.40 (4 H, m), 0.75-0.95 (2 H, m);HPLC-MS 1: 1.75 min, m/z 435[M + H]+ |
| 1-7 | 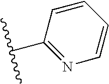 | CH₂ | H | 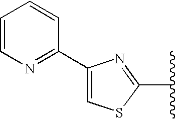 | 1H NMR (400 MHz, DMSO-d6): δ 8.61(1 H, d), 8.37 (1 H, d), 8.19-7.97(4 H, m), 7.87-7.80 (1 H, td), 7.58 (1 H,br.s.), 7.50 (1 H, br.s.), 7.15 (1 H, dd),4.02 (2 H, s), 3.51-3.27 (3 H, m),1.99 (2 H, d), 1.85 (4 H, dd), 1.78-1.62(3 H, m), 1.35-1.21 (2 H, m);HPLC-MS: 0.62 min, 422 [M + H]⁺ |
| 1-8 | 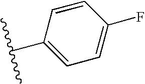 | CH₂ | H | 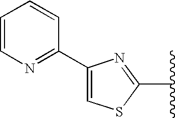 | 1H NMR (500 MHz, CDCl₃) δ 1.18-1.31(m, 2 H) 1.79-1.89 (m, 1 H) 1.85-1.94(td, 2 H) 1.99-2.09 (m, 4 H) 3.30(t, 2 H) 3.79 (s, 2 H) 5.23 (s, 1 H)7.08 (t, 2 H) 7.18-7.21 (m, 1 H) 7.30(s, 1 H) 7.53 (dd, 2 H) 7.73 (t, 1 H)7.90 (d, 1 H) 8.59 (d, 1 H);UPLC-MS: 0.65 min, 439 [M + H]+ |
| 1-9 | 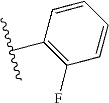 | CH₂ | H | 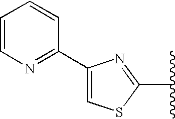 | 1H NMR (400 MHz, MeOH-d4) δ1.23-1.35 (m, 2 H) 1.80-1.94 (m, 3 H)1.96-2.06 (m, 2H) 2.09-2.18 (m, 2 H)3.32 (s, 4 H) 3.36 (s, 2 H) 3.44 (d, 2 H)3.92 (s, 2 H) 7.18-7.29 (m, 2 H)7.31-7.40 (m, 1 H) 7.52 (t, 1 H) 7.80-7.92(m, 2 H) 8.38-8.54 (m, 2 H)8.67 (d, 1 H)UPLC-MS: 0.62 min, 439 [M + H]+ |
| 1-10 | 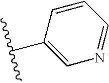 | CH₂ | H | 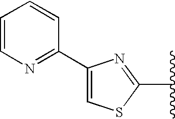 | 1H NMR (400 MHz, DMSO-d6):δ 8.70-8.66 (d, 1H), 8.54-8.50 (d,1 H), 8.40-8.30 (m, 4 H), 7.92-7.67 (m,3 H), 3.90 (s, 2H), 3.37 (br s, 1 H),2.21-1.20 (m, 9 H);HPLC-MS: 1.33 min, 422 [M + H]+ |
| 1-11 | 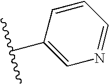 | CH₂ | H | 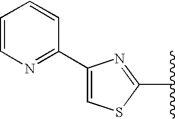 | ¹H NMR (400 MHz, DMSO-d6):δ 8.95 (d, 1H), 8.67 (d, 1H), 8.50 (dd,1 H), 8.40-5.23 (m, 3 H), 7.85-7.62 (m,3 H), 4.03 (s, 2 H), 3.34 (br s, 2 H),2.07-0.82 (m, 9 H);UPLC-MS: 0.47 min, 422 [M + H]+ |
| 1-12 | 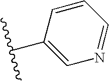 | CH₂ | H | 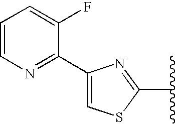 | ¹H NMR (400 MHz, DMSO-d6):δ 8.94 (d, 1 H), 8.55-8.48 (m, 2 H),8.35 (m, 1 H), 7.95-7.89 (m, 1 H), 7.79-7.74(m, 1 H), 7.58-7.52 (m, 1 H), 7.43(s, 1 H), 4.02 (s, 2 H), 3.31 (m, 2 H),2.06-1.67 (m, 7 H), 1.34-1.17 (m, 2 H);UPLC-MS: 0.54 min, 440 [M + H]+ |
| 1-13 | 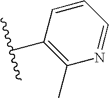 | CH₂ | H | 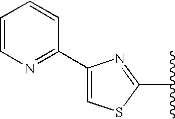 | ¹H NMR (400 MHz, DMSO-d6):δ 8.69-8.58 (m, 2 H), 8.36-8.22 (m,3 H), 7.87-7.81 (m, 1 H), 7.74-7.64 (m,2 H), 3.92 (s, 2 H), 3.32 (br s, 2 H),2.14-1.68 (m, 7 H), 1.27-1.13 (m, 2 H);UPLC-MS: 0.47 min, 436 [M + H]+ |
| 1-14 | 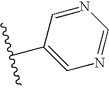 | CH₂ | H | 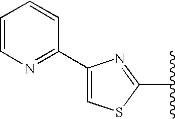 | ¹H NMR (400 MHz, DMSO-d6): δ9.04 (s, 2 H), 8.94 (s, 1 H), 8.69-8.65(m, 1 H), 8.35-8.24 (m, 2 H), 7.82 (m,1 H), 7.67 (m, 1 H), 4.00 (s, 2 H), 3.33(br s, 2 H), 2.08-1.65 (m, 7 H),1.32-1.19 (m, 2 H);UPLC-MS: 0.50 min, 423 [M + H]+ |
| 1-15 | 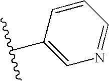 | CH₂ | Me | 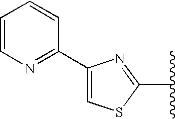 | 1H-NMR (400 MHz, DMSO-d6): δ8.71 (1 H, d), 8.31-8.56 (4 H, m), 8.09(1 H, d), 8.05 (1 H, s), 7.73-7.89 (2 H,m), 7.14 (1 H, dd), 3.95 (2 H, s), 3.43(2 H, s), 1.77-1.97 (4 H, m), 1.58-1.72(2 H, m), 1.33-1.47 (2 H, m), 1.04 (3 H,s);UPLC-MS: 0.62 min, 436 [M + H]+,218 [M + 2 H]2+ |
| 1-16 | 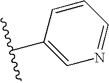 | CH₂ | Me | 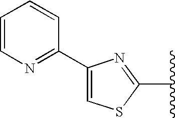 | 1H-NMR (400 MHz, CDCl₃): δ 8.68(1 H, d), 8.38 (1 H, d), 8.33 (1 H, brs),8.22 (1 H, brs), 8.1 (2 H, d), 7.80-7.94(2 H, m), 7.71 (1 H, brs), 7.16 (1 H, dd),3.98 (2 H, s), 3.47 (2 H, s), 1.75-2.01(4 H, m), 1.43-1.61 (4 H, m), 1.03 (3 H,s);UPLC-MS: 0.63 min, 436 [M + H]+,218 [M + 2 H]2+ |
| 1-17 | 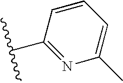 | CH₂ | H | 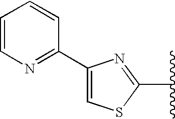 | 1H-NMR (400 MHz, DMSO-d6): δ1.20-1.33 (m, 2 H) 1.63-1.91 (m, 5 H)1.95-2.04 (m, 2 H) 2.44 (s, 3 H) 3.33-3.40(m, 2 H) 4.00 (s, 2 H) 7.01 (d,1 H) 7.64-7.93 (m, 4 H) 8.25-8.37 (m,2 H) 8.67 (d, 1 H);UPLC-MS: 0.66 min, 436 [M + H]+ |
| 1-18 | 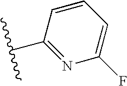 | CH₂ | H | 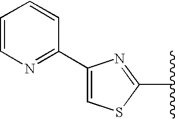 | 1H-NMR (400 MHz, DMSO-d6): δ1.18-1.37 (m, 2 H) 1.62-1.78 (m, 3 H)1.80-1.92 (m, 2 H) 1.95-2.07 (m, 2 H)3.31-3.37 (m, 2 H) 3.98 (s, 2 H) 6.85-6.97(m, 1 H) 7.51-7.80 (m, 2 H) 7.96-8.06(m, 2 H) 8.08-8.33 (m, 2 H)8.60-8.68 (m, 1 H);HPLC-MS: 2.018 min, 440 [M + H]+ |
| 1-19 | 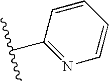 | CHMe | H | 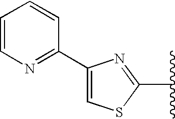 | 1H-NMR (400 MHz, DMSO-d6): δ8.70 (1 H, d), 8.22-8.44 (3 H, m), 8.11(1 H, d), 7.96 (1 H, brs), 7.81-7.88 (1 H,m), 7.76 (1 H, brs), 7.15 (1 H, dd), 4.02(2 H, s), 3.95-4.07 (1 H, m), 1.93-2.07(2 H, m), 1.80-1.92 (2 H, m), 1.53-1.77(3 H, m), 1.15-1.39 (2 H, m), 1.23 (3 H,s);UPLC-MS: 0.62 min, 436 [M + H]+,218 [M + 2 H]2+ |
| 1-20 | 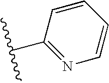 | CH₂ | H | 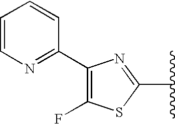 | ¹H NMR (400 MHz, CDCl₃): δ8.67-8.72 (1 H, m),8.31-8.35 (1 H, m), 8.23-8.30(1 H, m), 7.74-7.83 (1 H, m), 7.68-7.75(2 H, m), 7.16-7.22 (1 H, m), 7.01-7.07(1 H, m), 5.03-5.17 (1 H, br. s.),4.05 (2 H, s), 3.14-3.22 (2 H, d), 2.06-1.94(3 H, m), 1.94-1.79 (2 H, m),1.79-1.63 (4 H, m);HPLC-MS: 4.39 min, 440 [M + H]+ |

### Example 2

### Preparation of Compounds of Formula (IIb)

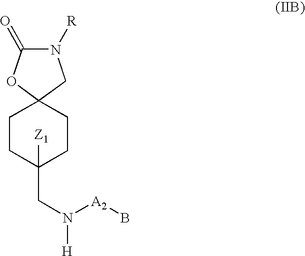

### Example 2-1

### (Trans)-8-({[1-(2-fluorophenyl)-1H-pyrazol-3-yl]amino}methyl)-3-(2-fluoro-3-pyridinyl)-1-oxa-3-azaspiro[4.5]decan-2-one bis hydrochloride

To (trans)-8-({[1-(2-fluorophenyl)-1H-pyrazol-3-yl]amino}methyl)-1-oxa-3-azaspiro[4.5]-decan-2-one (Intermediate 19, 50 mg, 0.145 mmol) and 3-bromo-2-fluoropyridine (Intermediate 14, 30.7 mg, 0.174 mmol) in 1,4-dioxane (2 ml) were added copper(I) iodide (27.7 mg, 0.145 mmol), trans 1,2-cyclohexanediamine (0.035 ml, 0.290 mmol) and potassium phosphate (154 mg, 0.726 mmol). The mixture obtained was stirred at 120° C. for 3 h. 1,4-dioxane was evaporated using a V10 Biotage and the crude obtained was dissolved in dichloromethane (8 ml) and filtered. The solution obtained was evaporated and the crude purified on KP—NH cartridge eluting with a mixture of cyclohexane/EtOAc. The desired compound (trans)-8-({[1-(2-fluorophenyl)-1H-pyrazol-3-yl]amino}methyl)-3-(2-fluoro-3-pyridinyl)-1-oxa-3-azaspiro[4.5]decan-2-one was eluted with ca 15% EtOAc (40 mg).

1H-NMR (400 MHz, CDCl₃): δ 8.13-8.19 (1H, m), 8.06-8.10 (1H, m), 7.83-7.89 (2H, m), 7.10-7.29 (4H, m), 5.82 (1H, d), 3.90 (2H, d), 3.15-3.21 (2H, m), 2.01-2.10 (4H, m), 1.83-1.94 (2H, m), 1.71-1.82 (1H, m), 1.12-1.25 (2H, m); UPLC-MS: 0.76 min, 440 [M+H]+.

This was dissolved in dichloromethane and 2.1 eq of a solution 1M HCl in diethylether was added to give the title compound (40 mg).

### Example 2-2

### (Trans)-8-({[1-(2-fluorophenyl)-1H-pyrazol-3-yl]amino}methyl)-3-(3-pyridazinyl)-1-oxa-3-azaspiro[4.5]decan-2-one hydrochloride

(Trans)-8-({[1-(2-fluorophenyl)-1H-pyrazol-3-yl]amino}methyl)-1-oxa-3-azaspiro[4.5]decan-2-one (prepared in a similar way to Intermediate 19, 50 mg, 0.145 mmol), 3-chloropyridazine (commercially available) (33.3 mg, 0.290 mmol), copper(I) iodide (27.7 mg, 0.145 mmol), potassium phosphate (154 mg, 0.726 mmol) and trans-1,2-diaminocyclohexane (0.017 ml, 0.145 mmol) were collected and shaken at 120° C. for 13 h. Solvent was removed, the crude was rinsed with dichloromethane and filtered, the resulting crude was immediately purified with Biotage SP1, over a KP—NH 25M column with a gradient of cyclohexane and ethyl acetate. The required compound (trans)-8-({[1-(2-fluorophenyl)-1H-pyrazol-3-yl]amino}methyl)-3-(3-pyridazinyl)-1-oxa-3-azaspiro[4.5]decan-2-one was eluted with ca 40% EtOAc and recovered as a colourless oil (50 mg).

1H-NMR (400 MHz, CDCl₃): δ 8.96 (1H, dd), 8.56 (1H, dd), 7.83-7.91 (2H, m), 7.49 (1H, dd), 7.10-7.24 (3H, m), 5.82 (1H, d), 4.22 (2H, s), 3.95 (1H, brs), 3.18 (2H, brm), 2.00-2.09 (2H, m), 1.89 (2H, td), 1.71-1.83 (1H, m), 1.18-1.31 (2H, m);

This was then reacted with 1.0 equiv. of 1.0 M HCl in Et₂O to afford the title compound (49 mg) as a colourless solid.

### Example 2-3

### (Trans)-8-({[1-(2-fluorophenyl)-1H-pyrazol-3-yl]amino}methyl)-3-(1-methyl-1H-pyrazol-3-yl)-1-oxa-3-azaspiro[4.5]decan-2-one dihydrochloride

1-(2-Fluorophenyl)-1H-pyrazol-3-amine (27 mg, 0.152 mmol) and trans 3-(1-methyl-1H-pyrazol-3-yl)-2-oxo-1-oxa-3-azaspiro[4.5]decane-8-carbaldehyde (Intermediate 23, 40.1 mg, 0.152 mmol) were mixed in dichloromethane (2 ml) at rt under nitrogen atmosphere. Titanium(IV) tetraisopropoxide (0.089 ml, 0.305 mmol) was added and the mixture was stirred for 18 hours. Sodium borohydride (17.30 mg, 0.457 mmol) was added and the reaction mixture was diluted with ethanol (2 ml). After stirring for 24 hours, the mixture was quenched with saturated aqueous sodium hydrogencarbonate (1 ml) and diluted with dichloromethane (40 ml). The organic phase was washed with saturated aqueous sodium hydrogencarbonate (10 ml) and brine (10 ml), then passed through a hydrophobic PTFE frit and evaporated. The crude was purified on NH-modified silica (Biotage) eluting with cyclohexane/ethyl acetate: 9/1 to 3/7. The desired product eluted at cyclohexane/ethyl acetate: 1/1. 49.0 mg of the target product (trans)-8-({[1-(2-fluorophenyl)-1H-pyrazol-3-yl]amino}methyl)-3-(1-methyl-1H-pyrazol-3-yl)-1-oxa-3-azaspiro[4.5]decan-2-one were isolated.

1H-NMR (400 MHz, CDCl₃): δ 7.88 (1H, dd), 7.85 (1H, t), 7.29 (1H, d), 7.08-7.25 (3H, m), 6.65 (1H, d), 5.82 (1H, d), 3.88 (2H, s), 3.84 (3H, s), 3.17 (2H, t), 1.96-2.06 (4H, m), 1.80-1.91 (2H, m), 1.68-1.80 (1H, m), 1.15-1.25 (2H, m); UPLC-MS: 0.75 min, 425 [M+H]+.

This was dissolved in dichloromethane and treated with 1M hydrogen chloride in diethyl ether. The solvents were stripped off and the resulting solid dried at 45° C. under high vacuum overnight to give the title compound (49.5 mg).

### Example 2-4

### (Trans)-8-({[1-(2-fluorophenyl)-1H-pyrazol-3-yl]amino}methyl)-3-[5-(trifluoromethyl)-3-Pyridinyl]-1-oxa-3-azaspiro[4.5]decan-2-one dihydrochloride

The title compound was made in a similar fashion to the preparation of Example 2-3 replacing (trans)-3-(1-methyl-1H-pyrazol-3-yl)-2-oxo-1-oxa-3-azaspiro[4.5]decane-8-carbaldehyde with (trans)-2-oxo-3-[5-(trifluoromethyl)-3-pyridinyl]-1-oxa-3-azaspiro-[4.5]decane-8-carbaldehyde (Intermediate 35, 40.8 mg, 0.124 mmol) to give the title compound (47 mg).

### Example 2-5

### (Trans)-8-({[1-(2-fluorophenyl)-1H-pyrazol-3-yl]amino}methyl)-3-(2-pyrazinyl)-1-oxa-3-azaspiro[4.5]decan-2-one hydrochloride

The title compound was made in a similar fashion to the preparation of Example 2-3 replacing (trans)-3-(1-methyl-1H-pyrazol-3-yl)-2-oxo-1-oxa-3-azaspiro[4.5]decane-8-carbaldehyde with (trans)-2-oxo-3-(2-pyrazinyl)-1-oxa-3-azaspiro[4.5]decane-8-carbaldehyde (Intermediate 38, 40.6 mg, 0.155 mmol) to give the title compound (37 mg).

### Example 2-6

### (Trans)-3-(2,1,3-benzothiadiazol-5-yl)-8-({[1-(2-fluorophenyl)-1H-pyrazol-3-yl]amino}methyl)-1-oxa-3-azaspiro[4.5]decan-2-one hydrochloride

The title compound was made in a similar fashion to the preparation of Example 2-2 replacing 3-chloropyridazine with 5-bromo-2,1,3-benzothiadiazole (18.74 mg, 0.087 mmol) to give the title compound (32.5 mg).

### Example 2-7

### (Trans)-3-(1,3-benzodioxol-5-yl)-8-({[1-(2-fluorophenyl)-1H-pyrazol-3-yl]amino}methyl)-1-oxa-3-azaspiro[4.5]decan-2-one hydrochloride

The title compound was made in a similar fashion to the preparation of Example 2-2 replacing 3-chloropyridazine with 5-bromo-1,3-benzodioxole (10.49 μl, 0.087 mmol) to give the title compound (32 mg).

### Example 2-8

### (Trans)-8-({[1-(2-fluorophenyl)-1H-pyrazol-3-yl]amino}methyl)-3-[2-(methyloxy)-5-pyrimidinyl]-1-oxa-3-azaspiro[4.5]decan-2-one hydrochloride

The title compound was made in a similar fashion to the preparation of Example 2-2 replacing 3-chloropyridazine with 5-bromo-2-(methyloxy)pyrimidine (16.47 mg, 0.087 mmol) to give the title compound (33 mg).

### Example 2-9

### (Trans)-8-({[1-(2-fluorophenyl)-1H-pyrazol-3-yl]amino}methyl)-3-(1-oxido-3-pyridinyl)-1-oxa-3-azaspiro[4.5]decan-2-one

The title compound was made in a similar fashion to the preparation of Example 2-2 replacing 3-chloropyridazine with 3-bromopyridine 1-oxide (15.16 mg, 0.087 mmol) to give the title compound (27.2 mg).

### Example 2-10

### (Trans)-8-({[1-(2-fluorophenyl)-1H-pyrazol-3-yl]amino}methyl)-3-(2-methyl-3-pyridinyl)-1-oxa-3-azaspiro[4.5]decan-2-one hydrochloride

The title compound was made in a similar fashion to the preparation of Example 2-3 replacing (trans)-3-(1-methyl-1H-pyrazol-3-yl)-2-oxo-1-oxa-3-azaspiro[4.5]decane-8-carbaldehyde with (trans)-3-(2-methyl-3-pyridinyl)-2-oxo-1-oxa-3-azaspiro[4.5]decane-8-carbaldehyde (Intermediate 50, 50 mg, 0.182 mmol) to afford the title compound as a colourless solid (66 mg).

### Example 2-11

### (Trans)-8-({[1-(2-fluorophenyl)-1H-pyrazol-3-yl]amino}methyl)-3-(5-pyrimidinyl)-1-oxa-3-azaspiro[4.5]decan-2-one dihydrochloride

The title compound was made in a similar fashion to the preparation of Example 2-3 replacing (trans)-3-(1-methyl-1H-pyrazol-3-yl)-2-oxo-1-oxa-3-azaspiro[4.5]decane-8-carbaldehyde with (trans)-2-oxo-3-(5-pyrimidinyl)-1-oxa-3-azaspiro[4.5]decane-8-carbaldehyde (Intermediate 53, 20 mg, 0.077 mmol) to give the title compound as a colourless solid (21 mg).

### Example 2-12

### (Trans)-8-({[1-(2-fluorophenyl)-1H-pyrazol-3-yl]amino}methyl)-3-(5-methyl-2-pyridinyl)-1-oxa-3-azaspiro[4.5]decan-2-one dihydrochloride

The title compound was made in a similar fashion to the preparation of Example 2-2 replacing 3-chloropyridazine with 2-bromo-4-methylpyridine (14.99 mg, 0.087 mmol) to afford the title compound as a colourless solid (43 mg).

### Example 2-13

### (Trans)-8-({[1-(2-fluorophenyl)-1H-pyrazol-3-yl]amino}methyl)-3-(6-methyl-3-pyridinyl)-1-oxa-3-azaspiro[4.5]decan-2-one dihydrochloride

The title compound was made in a similar fashion to the preparation of Example 2-2 replacing 3-chloropyridazine with 5-bromo-2-methylpyridine (14.99 mg, 0.087 mmol) to afford the title compound as a colourless solid (40 mg).

### Example 2-14

### (Trans)-8-({[1-(2-fluorophenyl)-1H-pyrazol-3-yl]amino}methyl)-3-(2-methyl-4-pyridinyl)-1-oxa-3-azaspiro[4.5]decan-2-one hydrochloride

The title compound was made in a similar fashion to the preparation of Example 2-2 replacing 3-chloropyridazine with 4-bromo-2-methylpyridine (15.1 mg, 0.088 mmol) to afford the title compound (15 mg).

### Example 2-15

### (Trans)-8-({[1-(2-fluorophenyl)-1H-pyrazol-3-yl]amino}methyl)-3-[6-(methyloxy)-3-pyridinyl]-1-oxa-3-azaspiro[4.5]decan-2-one hydrochloride

The title compound was made in a similar fashion to the preparation of Example 2-2 replacing 3-chloropyridazine with 5-bromo-2-(methyloxy)pyridine (16.5 mg, 0.088 mmol) to afford the title compound (14 mg).

### Example 2-16

### (Trans)-8-({[1-(2-fluorophenyl)-1H-pyrazol-3-yl]amino}methyl)-3-(6-fluoro-3-pyridinyl)-1-oxa-3-azaspiro[4.5]decan-2-one hydrochloride

The title compound was made in a similar fashion to the preparation of Example 2-2 replacing 3-chloropyridazine with 5-bromo-2-fluoropyridine (12.3 mg, 0.070 mmol) to afford the title compound as a colourless solid (18 mg).

### Example 2-17

### (Trans)-8-({[1-(2-fluorophenyl)-1H-pyrazol-3-yl]amino}methyl)-3-imidazo[1,2-a]pyridin-6-yl-1-oxa-3-azaspiro[4.5]decan-2-one hydrochloride

The title compound was made in a similar fashion to the preparation of Example 2-2 replacing 3-chloropyridazine with 6-bromoimidazo[1,2-a]pyridine (17.3 mg, 0.088 mmol) to afford the title compound (21.6 mg).

### Example 2-18

### (Trans)-3-(3-fluoro-6-methyl-2-pyridinyl)-8-({[1-(2-fluorophenyl)-1H-pyrazol-3-yl]amino}methyl)-1-oxa-3-azaspiro[4.5]decan-2-one hydrochloride

The title compound was made in a similar fashion to the preparation of Example 2-2 replacing 3-chloropyridazine with 2-bromo-3-fluoro-6-methylpyridine (17.3 mg, 0.088 mmol) to afford the title compound (25 mg).

### Example 2-19

### (Trans)-8-({[1-(2-fluorophenyl)-1H-pyrazol-3-yl]amino}methyl)-3-(1,3-thiazol-2-yl)-1-oxa-3-azaspiro[4.5]decan-2-one hydrochloride

The title compound was made in a similar fashion to the preparation of Example 2-2 replacing 3-chloropyridazine with 2-bromo-1,3-thiazole (14.4 mg, 0.088 mmol) to afford the title compound (16 mg).

### Example 2-20

### 4-[(Trans)-8-({[1-(2-fluorophenyl)-1H-pyrazol-3-yl]amino}methyl)-2-oxo-1-oxa-3-azaspiro[4.5]dec-3-yl]benzonitrile hydrochloride

The title compound was made in a similar fashion to the preparation of Example 2-2 replacing 3-chloropyridazine with 4-bromobenzonitrile (16.0 mg, 0.088 mmol) to afford the title compound (20 mg).

### Example 2-21

### 3-[(Trans)-8-({[1-(2-fluorophenyl)-1H-pyrazol-3-yl]amino}methyl)-2-oxo-1-oxa-3-azaspiro[4.5]dec-3-yl]benzonitrile hydrochloride

The title compound was made in a similar fashion to the preparation of Example 2-2 replacing 3-chloropyridazine with 3-bromobenzonitrile (16.0 mg, 0.088 mmol) to afford the title compound (15.5 mg).

### Example 2-22

### 3-[(Trans)-8-({[1-(2-fluorophenyl)-1H-pyrazol-3-yl]amino}methyl)-2-oxo-1-oxa-3-azaspiro[4.5]dec-3-yl]benzonitrile hydrochloride

The title compound was made in a similar fashion to the preparation of Example 2-2 replacing 3-chloropyridazine with 2-bromo-1,3-benzothiazole (18.8 mg, 0.088 mmol) to afford the title compound (17.6 mg).

### Example 2-23

### (Trans)-8-({[1-(2-fluorophenyl)-1H-pyrazol-3-yl]amino}methyl)-3-(5-fluoro-3-pyridinyl)-1-oxa-3-azaspiro[4.5]decan-2-one dihydrochloride

The title compound was made in a similar fashion to the preparation of Example 2-2 replacing 3-chloropyridazine with 3-bromo-5-fluoropyridine (15.33 mg, 0.087 mmol) to afford the title compound (23 mg).

### Example 2-24

### (Trans)-8-({[1-(2-fluorophenyl)-1H-pyrazol-3-yl]amino}methyl)-3-(1-methyl-1H-imidazol-5-yl)-1-oxa-3-azaspiro[4.5]decan-2-one hydrochloride

The title compound was made in a similar fashion to the preparation of Example 2-2 replacing 3-chloropyridazine with 5-bromo-1-methyl-1H-imidazole (17.0 mg, 0.088 mmol) to afford the title compound (14.3 mg).

### Example 2-25

### (Trans)-8-({[1-(2-fluorophenyl)-1H-pyrazol-3-yl]amino}methyl)-3-imidazo[1,2-a]pyrazin-3-yl-1-oxa-3-azaspiro[4.5]decan-2-one hydrochloride

The title compound was made in a similar fashion to the preparation of Example 2-2 replacing 3-chloropyridazine with 3-bromoimidazo[1,2-a]pyrazine (20.9 mg, 0.088 mmol) to afford the title compound (17.5 mg).

### Example 2-26

### (Trans)-8-({[1-(2-fluorophenyl)-1H-pyrazol-3-yl]amino}methyl)-3-(1-methyl-6-oxo-1,6-dihydro-3-pyridinyl)-1-oxa-3-azaspiro[4.5]decan-2-one hydrochloride

The title compound was made in a similar fashion to the preparation of Example 2-2 replacing 3-chloropyridazine with 5-bromo-1-methyl-2(1H)-pyridinone (19.9 mg, 0.088 mmol) to afford the title compound (12.5 mg).

### Example 2-27

### (Trans)-8-({[1-(2-fluorophenyl)-1H-pyrazol-3-yl]amino}methyl)-3-imidazo[1,2-a]pyridin-7-yl-1-oxa-3-azaspiro[4.5]decan-2-one hydrochloride

The title compound was made in a similar fashion to the preparation of Example 2-2 replacing 3-chloropyridazine with 7-bromoimidazo[1,2-a]pyridine (20.8 mg, 0.088 mmol) to afford the title compound (16 mg).

### Example 2-28

### (Trans)-3-(2,1,3-benzoxadiazol-5-yl)-8-({[1-(2-fluorophenyl)-1H-pyrazol-3-yl]amino}methyl)-1-oxa-3-azaspiro[4.5]decan-2-one hydrochloride

The title compound was made in a similar fashion to the preparation of Example 2-2 replacing 3-chloropyridazine with 5-bromo-2,1,3-benzoxadiazole (21.0 mg, 0.088 mmol) to afford the title compound (13 mg).

### Example 2-29

### (Trans)-8-({[1-(2-fluorophenyl)-1H-pyrazol-3-yl]amino}methyl)-3-(3-methyl-5-isothiazolyl)-1-oxa-3-azaspiro[4.5]decan-2-one hydrochloride

The title compound was made in a similar fashion to the preparation of Example 2-2 replacing 3-chloropyridazine with 5-bromo-3-methylisothiazole (18.8 mg, 0.088 mmol) to afford the title compound (17.8 mg).

### Example 2-30

### (Trans)-8-({[1-(2-fluorophenyl)-1H-pyrazol-3-yl]amino}methyl)-3-(1-methyl-1H-imidazol-2-yl)-1-oxa-3-azaspiro[4.5]decan-2-one hydrochloride

The title compound was made in a similar fashion to the preparation of Example 2-2 replacing 3-chloropyridazine with 2-iodo-1-methyl-1H-imidazole (22.0 mg, 0.088 mmol) to afford the title compound (12.7 mg).

### Example 2-31

### (Trans)-8-({[1-(2-fluorophenyl)-1H-pyrazol-3-yl]amino}methyl)-3-(2-pyrimidinyl)-1-oxa-3-azaspiro[4.5]decan-2-one hydrochloride

The title compound was made in a similar fashion to the preparation of Example 2-2 replacing 3-chloropyridazine with 2-bromopyrimidine (16.8 mg, 0.088 mmol) to afford the title compound (5.7 mg).

### Example 2-32

### (Trans)-3-(2-fluoro-6-methyl-3-pyridinyl)-8-({[1-(2-fluorophenyl)-1H-pyrazol-3-yl]amino}methyl)-1-oxa-3-azaspiro[4.5]decan-2-one hydrochloride

The title compound was made in a similar fashion to the preparation of Example 2-2 replacing 3-chloropyridazine with 3-bromo-2-fluoro-6-methylpyridine (20.1 mg, 0.088 mmol) to afford the title compound (21.0 mg).

### Example 2-33

### (Trans)-8-({[1-(2-fluorophenyl)-1H-pyrazol-3-yl]amino}methyl)-3-(2-methyl-5-pyrimidinyl)-1-oxa-3-azaspiro[4.5]decan-2-one hydrochloride

The title compound was made in a similar fashion to the preparation of Example 2-2 replacing 3-chloropyridazine with 5-bromo-2-methylpyrimidine (18.3 mg, 0.088 mmol) to afford the title compound (18.3 mg).

### Example 2-34

### (Trans)-8-({[1-(2-fluorophenyl)-1H-pyrazol-3-yl]amino}methyl)-3-(2-methyl-1,3-thiazol-4-yl)-1-oxa-3-azaspiro[4.5]decan-2-one hydrochloride

The title compound was made in a similar fashion to the preparation of Example 2-2 replacing 3-chloropyridazine with 4-bromo-2-methyl-1,3-thiazole (18.8 mg, 0.088 mmol) to afford the title compound (21 mg).

### Example 2-35

### (Trans)-8-({[1-(2-fluorophenyl)-1H-pyrazol-3-yl]amino}methyl)-3-[2-(trifluoromethyl)-5-pyrimidinyl]-1-oxa-3-azaspiro[4.5]decan-2-one hydrochloride

The title compound was made in a similar fashion to the preparation of Example 2-2 replacing 3-chloropyridazine with 5-bromo-2-(trifluoromethyl)pyrimidine (16.48 mg, 0.073 mmol) to afford the title compound (18 mg).

### Example 2-36

### (Trans)-8-({[1-(2-fluorophenyl)-1H-pyrazol-3-yl]amino}methyl)-3-(2-fluoro-4-pyridinyl)-1-oxa-3-azaspiro[4.5]decan-2-one hydrochloride hydrochloride

The title compound was made in a similar fashion to the preparation of Example 2-2 replacing 3-chloropyridazine with 4-bromo-2-fluoropyridine (15.42 mg, 0.073 mmol) to afford the title compound (21 mg).

### Example 2-37

### (Trans)-3-(2,6-dimethyl-4-pyridinyl)-8-({[1-(2-fluorophenyl)-1H-pyrazol-3-yl]amino}methyl)-1-oxa-3-azaspiro[4.5]decan-2-one dihydrochloride

The title compound was made in a similar fashion to the preparation of Example 2-2 replacing 3-chloropyridazine with 4-bromo-2,6-dimethylpyridine (13.51 mg, 0.073 mmol) to afford the title compound (19 mg).

### Example 2-38

### (Trans)-8-({[1-(2-fluorophenyl)-1H-pyrazol-3-yl]amino}methyl)-3-(4-pyridazinyl)-1-oxa-3-azaspiro[4.5]decan-2-one hydrochloride

The title compound was made in a similar fashion to the preparation of Example 2-2 replacing 3-chloropyridazine with 4-bromopyridazine (13.85 mg, 0.087 mmol) to afford the title compound (10 mg).

### Example 2-39

### (Trans)-8-({[1-(2-fluorophenyl)-1H-pyrazol-3-yl]amino}methyl)-3-(5-methyl-1,3,4-thiadiazol-2-yl)-1-oxa-3-azaspiro[4.5]decan-2-one hydrochloride

The title compound was made in a similar fashion to the preparation of Example 2-2 replacing 3-chloropyridazine with 2-bromo-5-methyl-1,3,4-thiadiazole (15.60 mg, 0.087 mmol) to afford the title compound (15 mg).

### Example 2-40

### (Trans)-8-({[1-(2-fluorophenyl)-1H-pyrazol-3-yl]amino}methyl)-3-(3-pyridinyl)-1-oxa-3-azaspiro[4.5]decan-2-one hydrochloride

The title compound was made in a similar fashion to the preparation of Example 2-3 replacing (trans)-3-(1-methyl-1H-pyrazol-3-yl)-2-oxo-1-oxa-3-azaspiro[4.5]decane-8-carbaldehyde with (trans)-2-oxo-3-(3-pyridinyl)-1-oxa-3-azaspiro[4.5]decane-8-carbaldehyde (prepared in a similar fashion to Intermediate 44, 40 mg, 0.154 mmol) to afford the title compound (34 mg).

### Example 2-41

### (Trans)-8-({[1-(2-fluorophenyl)-1H-pyrazol-3-yl]amino}methyl)-3-(1H-pyrazol-3-yl)-1-oxa-3-azaspiro[4.5]decan-2-one hydrochloride

1.0 M HCl in diethyl ether (2 ml, 2.0 mmol) was added to a solution of (trans)-8-({[1-(2-fluorophenyl)-1H-pyrazol-3-yl]amino}methyl)-3-[1-(tetrahydro-2H-pyran-2-yl)-1H-pyrazol-3-yl]-1-oxa-3-azaspiro[4.5]decan-2-one (Intermediate 69, 45 mg, 0.091 mmol) in ethanol (2 ml) at room temperature under nitrogen. The resulting solution was left to stand for 1 hour then heated to 45° C. for 1 hour. Evaporate volatiles under reduced pressure. Dissolve residue in MeOH (1 ml) and load onto a 2 g SCX cartridge. Elute with MeOH and then a 2M solution of NH3 in MeOH. Combine the basic fractions and evaporate under reduced pressure. The residue was purified via Biotage (5%-20% MeOH/CH2Cl2; 12M NH column) to give (trans)-8-({[1-(2-fluorophenyl)-1H-pyrazol-3-yl]amino}methyl)-3-(1H-pyrazol-3-yl)-1-oxa-3-azaspiro[4.5]decan-2-one (N1015-52-1) (33 mg) as a colourless oil. 1H-NMR (400 MHz, CDCl₃): δ 10.10 (1H, brs), 7.76-7.93 (2H, m), 7.50 (1H, d), 7.08-7.25 (3H, m), 6.73 (1H, brs), 5.81 (1H, d), 4.15-4.29 (1H, m), 3.88 (2H, s), 3.15 (2H, t), 1.95-2.14 (4H, m), 1.67-1.94 (3H, m), 1.08-1.32 (2H, m); m/z 411 [M+H]+, 206 [M+2H]2+

This was dissolved in dichloromethane (2 ml) and MeOH (0.1 ml) and then treated with a 1.0M solution of HCl in diethyl ether (2.5 eg, 0.20 ml, 0.20 mmol). The resulting solution was left to stand for 30 minutes then evaporated under reduced pressure. The residue was triturated with diethyl ether (2 ml) and the solid collected by filtration. Dry under vacuum at 60° C. for 18 hours to give the title compound (14 mg) as a white solid.

### Example 2-42

### (Trans)-8-({[1-(2-fluorophenyl)-1H-pyrazol-3-yl]amino}methyl)-3-(1H-pyrazol-4-yl)-1-oxa-3-azaspiro[4.5]decan-2-one hydrochloride

The title compound was made in a similar fashion to the preparation of Example 2-41 using (trans)-8-({[1-(2-fluorophenyl)-1H-pyrazol-3-yl]amino}methyl)-3-[1-(tetrahydro-2H-pyran-2-yl)-1H-pyrazol-4-yl]-1-oxa-3-azaspiro[4.5]decan-2-one (Intermediate 70, 45 mg, 0.091 mmol) to give the title compound (34.3 mg).

### Example 2-43

### (Trans)-8-({[1-(2-fluorophenyl)-1H-pyrazol-3-yl]amino}methyl)-3-(2-pyridinyl)-1-oxa-3-azaspiro[4.5]decan-2-one hydrochloride

The title compound was made in a similar fashion to the preparation of Example 2-3 replacing (trans)-3-(1-methyl-1H-pyrazol-3-yl)-2-oxo-1-oxa-3-azaspiro[4.5]decane-8-carbaldehyde with (trans)-2-oxo-3-(2-pyridinyl)-1-oxa-3-azaspiro[4.5]decane-8-carbaldehyde (this may be prepared as described for intermediate 26, 150 mg, 0.576 mmol) to give the title compound (153 mg, 52%).

### Example 2-44

### 8-fluoro-8-({[1-(2-fluorophenyl)-1H-pyrazol-3-yl]amino}methyl)-3-(2-pyridinyl)-1-oxa-3-azaspiro[4.5]decan-2-one dihydrochloride

The title compound was made in a similar fashion to the preparation of Example 2-3 replacing (trans)-3-(1-methyl-1H-pyrazol-3-yl)-2-oxo-1-oxa-3-azaspiro[4.5]decane-8-carbaldehyde with 8-fluoro-2-oxo-3-(2-pyridinyl)-1-oxa-3-azaspiro[4.5]decane-8-carbaldehyde (Intermediate 75, 24 mg, 0.086 mmol) to afford the title compound (9 mg).

### Example 2-45

### 8-fluoro-8-({[1-(2-fluorophenyl)-1H-pyrazol-3-yl]amino}methyl)-3-(2-pyridinyl)-1-oxa-3-azaspiro[4.5]decan-2-one

The title compound was made in a similar fashion to the preparation of Example 2-3 replacing (trans)-3-(1-methyl-1H-pyrazol-3-yl)-2-oxo-1-oxa-3-azaspiro[4.5]decane-8-carbaldehyde with 8-fluoro-2-oxo-3-(2-pyridinyl)-1-oxa-3-azaspiro[4.5]decane-8-carbaldehyde (Intermediate 74, 55 mg, 0.198 mmol) without conversion of the free base to the hydrochloride salt to afford the title compound (31 mg).

### Example 2-46

### (Trans)-8-({[1-(2-fluorophenyl)-1H-pyrazol-3-yl]amino}methyl)-3-[1,2,4]triazolo[1,5-a]pyridin-6-yl-1-oxa-3-azaspiro[4.5]decan-2-one dihydrochloride

The title compound was made in a similar fashion to the preparation of Example 2-2 replacing 3-chloropyridazine with 6-bromo[1,2,4]triazolo[1,5-a]pyridine (28.7 mg, 0.145 mmol) to give the title compound (42.4 mg, 55%).

### Example 2-47

### (Trans)-8-({[1-(2-fluorophenyl)-1H-pyrazol-3-yl]amino}methyl)-3-[1,2,4]triazolo[4,3-a]pyridin-6-yl-1-oxa-3-azaspiro[4.5]decan-2-one dihydrochloride

The title compound was made in a similar fashion to the preparation of Example 2-2 replacing 3-chloropyridazine with 6-bromo[1,2,4]triazolo[4,3-a]pyridine (28.7 mg, 0.145 mmol) to give the title compound (28.4 mg, 36%).

### Example 2-48

### (Trans)-8-({[1-(2-fluorophenyl)-1H-pyrazol-3-yl]amino}methyl)-3-(1-methyl-1H-pyrazol-4-yl)-1-oxa-3-azaspiro[4.5]decan-2-one dihydrochloride

The title compound was made in a similar fashion to the preparation of Example 2-2 replacing 3-chloropyridazine with 4-iodo-1-methyl-1H-pyrazole (30.8 mg, 0.148 mmol) to give the title compound (39.7 mg, 54%).

### Example 2-49

### (Trans)-8-{[(5-phenyl-1H-pyrazol-3-yl)amino]methyl}-3-(3-pyridinyl)-1-oxa-3-azaspiro[4.5]decan-2-one hydrochloride

The title compound was made in a similar fashion to the preparation of Example 2-3 replacing (trans)-3-(1-methyl-1H-pyrazol-3-yl)-2-oxo-1-oxa-3-azaspiro[4.5]decane-8-carbaldehyde with (trans)-2-oxo-3-(3-pyridinyl)-1-oxa-3-azaspiro[4.5]decane-8-carbaldehyde (prepared in a similar fashion to Intermediate 44, 40 mg, 0.154 mmol) to give the title compound (40 mg).

### Example 2-50

### (Cis)-8-({[1-(2-fluorophenyl)-1H-pyrazol-3-yl]amino}methyl)-3-(3-pyridinyl)-1-oxa-3-azaspiro[4.5]decan-2-one hydrochloride

The title compound was made in a similar fashion to the preparation of Example 2-3 replacing (trans)-3-(1-methyl-1H-pyrazol-3-yl)-2-oxo-1-oxa-3-azaspiro[4.5]decane-8-carbaldehyde with (cis)-2-oxo-3-(3-pyridinyl)-1-oxa-3-azaspiro[4.5]decane-8-carbaldehyde (Intermediate 41, 55 mg, 0.211 mmol) to afford the title compound (56 mg).

### Example 2-51

### 1-(2-fluorophenyl)-3-({[(trans)-2-oxo-3-(2-pyridinyl)-1-oxa-3-azaspiro[4.5]dec-8-yl]methyl}-amino)-1H-pyrazole-4-carbonitrile

(Trans)-8-({[1-(2-fluorophenyl)-4-iodo-1H-pyrazol-3-yl]amino}methyl)-3-(2-pyridinyl)-1-oxa-3-azaspiro[4.5]decan-2-one (Intermediate 77, 56 mg, 0.102 mmol), copper(I) iodide (1.948 mg, 10.23 μmol) and KCN (7.99 mg, 0.123 mmol) were placed in a round bottomed flask. Then the flask was flushed with nitrogen three times and toluene (1 mL) was added followed by N,N′-dimethyl-1,2-ethanediamine (10.89 μL, 0.102 mmol). The solution was then heated at 110° C. for 30 h 20 min. 7.99 mg of KCN, 1.94 mg of CuI and 10.9 μL of N,N′-dimethyl-1,2-ethanediamine were added and the mixture was heated at 110° C. for an additional 18 h. 10 mL of a saturated solution of K₂CO₃ were added and the aqueous phase was extracted 3 times with 10 mL of AcOEt. The combined organic layers were washed with 10 mL of brine, dried over Na₂SO₄, filtered and evaporated to dryness. The resulting crude compound was then purified by flash chromatography (ISCO COMPANION, 12 g silica gel column) with the following gradient: A: Cyclohexane/B: AcOEt: 0% B for 1.4 min, 0% to 25% B in 14.3 min, 25% B for 2.9 min to give 15.8 mg of a colourless wax which was purified by MDAP to give the title compound (4.9 mg, 11%).

### Example 2-52

### (Trans)-8-({[1-(2-fluorophenyl)-4-(trifluoromethyl)-1H-pyrazol-3-yl]amino}methyl)-3-(2-Pyridinyl)-1-oxa-3-azaspiro[4.5]decan-2-one

Potassium fluoride (23.35 mg, 0.402 mmol) and copper(I) iodide (77 mg, 0.402 mmol) were placed in a flask under nitrogen. The solids were heated with a heating gun under high vacuum until a greenish color appeared. Then the mixture was allowed to cool to r.t. and a solution of (trans)-8-({[1-(2-fluorophenyl)-4-iodo-1H-pyrazol-3-yl]amino}methyl)-3-(2-pyridinyl)-1-oxa-3-azaspiro[4.5]decan-2-one (Intermediate 77, 200 mg, 0.365 mmol) in N,N-dimethylformamide (0.365 mL) and N-methyl-2-pyrrolidone (0.365 mL) was added followed by trifluoromethyltrimethylsilane (0.054 mL, 0.365 mmol) and the mixture was stirred at r.t. for 18 h 30 min. The mixture was cooled to r.t., diluted with 15 mL of concentrated NH4OH and extracted 4 times with 10 mL of AcOEt. The combined organic layers were washed with 15 mL of brine, dried over Na2SO4, filtered and evaporated to dryness. The residue was then purified by flash chromatography (ISCO COMPANION, 12 g silica gel column) with the following gradient: A: cyclohexane/B: AcOEt: 0% B for 2.1 min, 0% B to 25% B in 13.9 min, 25% B for 5.4 min, 25% B to 50% B in 5.4 min, 50% B for 3.2 min. and then by chiral preparative HPLC to afford the title compound as a white solid (3.7 mg, 2%).

### Example 2-53

### (Trans)-8-({[4-fluoro-1-(2-fluorophenyl)-1H-pyrazol-3-yl]amino}methyl)-3-(2-pyridinyl)-1-oxa-3-azaspiro[4.5]decan-2-one

(Trans)-2-oxo-3-(2-pyridinyl)-1-oxa-3-azaspiro[4.5]decane-8-carbaldehyde (this may be prepared as described for intermediate 26, 29.3 mg, 0.113 mmol) and 4-fluoro-1-(2-fluorophenyl)-1H-pyrazol-3-amine (Intermediate 78, 22 mg, 0.113 mmol) were dissolved in 1,2-dichloroethane (350 μL). Then titanium(IV) isopropoxide (66.1 μL, 0.225 mmol) was added and the mixture was stirred at 60° C. for 5 h30. The solution was cooled to r.t. and methanol (220 μL) was added followed by sodium borohydride (12.79 mg, 0.338 mmol). The mixture was stirred at r.t. for 16 h 10 min. 2 mL of a saturated solution of K2CO3 were added. The mixture was stirred at r.t. for 5 min, filtered and the cake was washed with 10 mL of AcOEt. The biphasic solution was transferred to a separatory funnel, the organic phase was kept and the aqueous phase was extracted with 5 mL of AcOEt. The combined organic layers were washed once with 5 mL of brine, dried over Na2SO4, filtered and evaporated to dryness. The resulting residue was then purified by flash chromatography (ISCO COMPANION, 12 g silica gel column) with the following gradient: A: Cyclohexane/B: AcOEt: 0% B for 1.8 min, 0% to 25% B in 17.9 min, 25% B for 3.6 min. Only the fractions corresponding to the desired compound were collected. Solvents were removed under reduced pressure and the compound was dissolved in 10 mL of DCM. The solution was passed over a 1 g SCX cartridge. The cartridge was then washed with 15 mL of DCM, 15 mL of MeOH and the compound was released with 10 mL of a solution of NH3 2M in MeOH. Solvents were removed under reduced pressure to give the title compound as a yellow film (20.2 mg, 40%).

### Example 2-54

### (Trans)-8-({[4-fluoro-1-(2-fluorophenyl)-1H-pyrazol-3-yl]amino}methyl)-3-(3-pyridazinyl)-1-oxa-3-azaspiro[4.5]decan-2-one

The title compound was made in a similar fashion to the preparation of Example 2-2 replacing (trans)-8-({[1-(2-fluorophenyl)-1H-pyrazol-3-yl]amino}methyl)-1-oxa-3-azaspiro[4.5]decan-2-one with (trans)-8-({[4-fluoro-1-(2-fluorophenyl)-1H-pyrazol-3-yl]amino}methyl)-1-oxa-3-azaspiro[4.5]decan-2-one (Intermediate 79, 50 mg, 0.138 mmol), to afford the title compound as a colourless film (22.1 mg, 34.5%).

### Example 2-55

### (Trans)-8-({[1-(2-fluorophenyl)-5-(trifluoromethyl)-1H-pyrazol-3-yl]amino}methyl)-3-(3-pyridazinyl)-1-oxa-3-azaspiro[4.5]decan-2-one

The title compound was made in a similar fashion to the preparation of Example 2-3 replacing (trans)-3-(1-methyl-1H-pyrazol-3-yl)-2-oxo-1-oxa-3-azaspiro[4.5]decane-8-carbaldehyde with (trans)-2-oxo-3-(3-pyridazinyl)-1-oxa-3-azaspiro[4.5]decane-8-carbaldehyde (Intermediate 82, 95 mg, 0.362 mmol) and 1-(2-fluorophenyl)-1H-pyrazol-3-amine with 1-(2-fluorophenyl)-5-(trifluoromethyl)-1H-pyrazol-3-amine (Intermediate 84, 97.6 mg, 0.398 mmol) to afford the title compound as a white solid (74.5 mg, 42%).

All the analytical data are set forth in the following Table 2-1 and in which R, Z₁, A₂ and B are:

| | |
|---|---|
| | (IIB) |
| 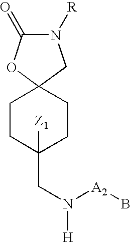 | |

| Cpd. | | | | |
|---|---|---|---|---|
| No. | R | Z₁ | A2—B | Analytical Data |
| 2-1 | 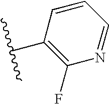 | H | 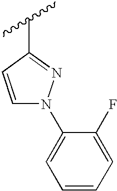 | 1H-NMR (400 MHz, DMSO-d6): δ 8.12-8.19(2 H, m), 7.88 (1 H, t), 7.77 (1 H, td),7.42-7.47 (1 H, m), 7.36 (1 H, ddd), 7.27(1 H, td), 7.16-7.24 (1 H, m), 5.84 (1 H, d),3.91 (2 H, s), 3.03 (2 H, d), 1.98-2.05 (2 H,m), 1.82-1.90 (2 H, m), 1.62-1.76 (3 H, m),1.10-1.25 (2 H, m);UPLC-MS: 0.76 min, 440 [M + H]+. |
| 2-2 | 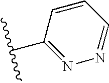 | H | 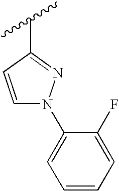 | 1H-NMR (400 MHz, DMSO-d6): δ 9.01(1 H, d), 8.37 (1 H, d), 7.89 (1 H, t), 7.71-7.80(2 H, m), 7.33-7.38 (1 H, m), 7.26(1 H, t), 7.17-7.22 (1 H, m), 5.87 (1 H, d),4.12 (2 H, s), 3.07 (2 H, d), 2.02 (2 H, d),1.87 (2 H, d), 1.64-1.74 (3 H, m), 1.20-1.30(2 H, m);UPLC-MS: 0.73 min, 423 [M + H]+ |
| 2-3 | 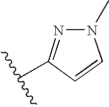 | H | 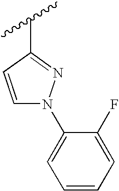 | 1H-NMR (400 MHz, DMSO-d6): δ 7.93(1 H, t), 7.78 (1 H, td), 7.63 (1 H, d), 7.38(1 H, ddd), 7.29 (1 H, td), 7.19-7.26 (1 H,m), 6.38 (1 H, d), 5.93 (1 H, d), 3.81 (2 H,s), 3.76 (3 H, s), 3.07 (2 H, d), 1.94 (2 H,d), 1.85 (2 H, dd), 1.66 (3 H, td), 1.14-1.39(3 H, m) |
| 2-4 | 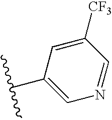 | H | 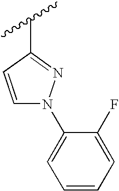 | 1H-NMR (400 MHz, DMSO-d6): δ 9.11(1 H, d), 8.72 (1 H, d), 8.40 (1 H, t), 7.93(1 H, t), 7.78 (1 H, td), 7.38 (1 H, ddd), 7.29(1 H, td), 7.19-7.26 (1 H, m), 5.91 (1 H, d),4.05 (2 H, s), 3.07 (2 H, d), 2.00 (2 H, d),1.89 (2 H, dd), 1.69 (3 H, td), 1.14-1.40(3 H, m) |
| 2-5 | 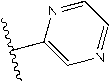 | H | 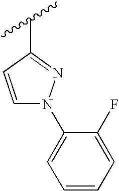 | 1H-NMR (400 MHz, DMSO-d6): δ 9.37(1 H, d), 8.44 (1 H, dd), 8.38 (1 H, dd), 7.92(1 H, t), 7.78 (1 H, td), 7.38 (1 H, ddd), 5.97(1 H, d), 3.99 (2 H, s), 3.09 (2 H, d), 2.02(2 H, d), 1.87 (2 H, dd), 1.70 (3 H, td),1.17-1.39 (3 H, m). |
| 2-6 | 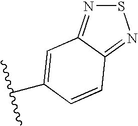 | H | 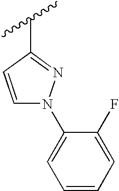 | ¹H NMR (400 MHz, DMSO-d6): δ 8.44(dd, 1 H), 8.12 (dd, 1 H), 7.95 (dd, 1 H),7.91 (t, 1 H), 7.79 (dt, 1 H), 7.40-7.18 (m,3 H), 5.87 (d, 1 H), 4.09 (s, 2 H), 3.87 (br s,1 H), 3.07 (d, 2 H), 2.08-1.60 (m, 7 H),1.33-1.19 (m, 2 H);UPLC-MS: 0.85 min, 479 [M + H]+. |
| 2-7 | 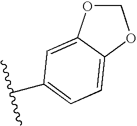 | H | 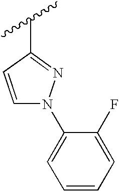 | ¹H NMR (400 MHz, DMSO-d6): δ 7.89 (t,1 H), 7.78 (dt, 1 H), 7.40-7.17 (m, 4 H),6.98-6.91 (m, 2 H), 6.02 (s, 2 H), 5.84 (d,1 H), 3.85 (s, 2 H), 3.79 (br s, 1 H), 3.02 (d,2 H), 1.98-1.58 (m, 7 H), 1.29-1.15 (m,2 H);UPLC-MS: 0.81 min, 465 [M + H]+ |
| 2-8 | 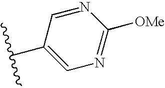 | H | 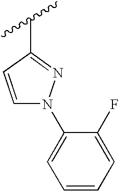 | ¹H NMR (400 MHz, DMSO-d6): δ 8.82 (s,2 H), 7.90 (t, 1 H), 7.78 (dt, 1 H), 7.40-7.18(m, 3 H), 5.85 (d, 1 H), 4.01 (br s, 1 H),3.94 (s, 2 H), 3.91 (s, 3 H), 3.05 (d, 2 H),2.04-1.61 9 m, 7 H), 1.32-1.09 (m, 2 H);UPLC-MS: 0.75 min, 453 [M + H]+ |
| 2-9 | 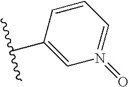 | H | 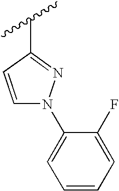 | ¹H NMR (400 MHz, CDCl₃): δ 8.44 (br s,2 H), 8.23 (m, 1 H,), 8.00 (d, 1 H), 7.87-7.77(m, 3 H), 7.32-7.10 (m, 3 H), 5.81 (d,1 H), 3.86 (m, 1 H), 3.72 (s, 2 H), 3.19 (t,2 H), 2.10-1.10 (m, 9 H);UPLC-MS: 0.65 min, 438 [M + H]+ |
| 2-10 | 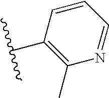 | H | 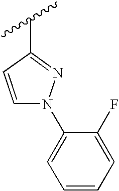 | ¹H NMR (400 MHz, DMSO-d6): δ 8.63(m, 1 H), 8.35-8.30 (m, 1 H), 7.89 (t, 1 H),7.78-7.70 (m, 2 H), 7.38-7.20 (m, 4 H),5.85 (d, 1 H), 3.91 (s, 2 H), 3.03 (d, 2 H),2.57 (s, 3 H), 2.21-1.70 (t m, 7 H), 1.12(m, 1 H);UPLC-MS: 0.67 min, 436 [M + H]+ |
| 2-11 | 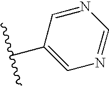 | H | 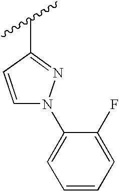 | ¹H NMR (400 MHz, DMSO-d6): δ 9.05 (s,2 H), 8.94 (s, 1 H), 7.89 (t, 1 H), 7.77 (dt,1 H), 7.40-7.19 (m, 3 H), 5.85 (d, 1 H), 3.09(s, 2 H), 3.05 (d, 2 H), 2.04-1.59 (m, 7 H),1.25-1.10 (m, 2 H);UPLC-MS: 0.71 min, 422 [M + H]+ |
| 2-12 | 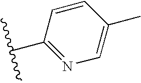 | H | 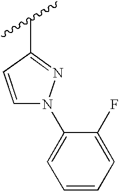 | ¹H NMR (400 MHz, DMSO-d6): δ 8.54 (d,1 H), 8.32 (s, 1 H), 7.95 (d, 1 H), 7.88-7.82(m, 2 H), 7.37-7.32 (m, 2 H), 7.30-7.24 (m,1 H), 7.27 (s, 1 H), 6.12 (br s, 1 H), 4.45 (s,2 H), 3.30 (s, 2 H), 2.44 (s, 3 H), 2.12-1.78(m, 7 H), 1.47-1.36 (m, 1 H);UPLC-MS: 0.84 min, 436 [M + H]+ |
| 2-13 | 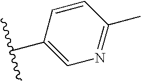 | H | 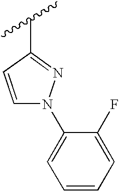 | ¹H NMR (400 MHz, DMSO-d6): δ 9.04 (d,1 H), 8.79 (s, 1 H), 7.88 (s, H), 7.81 (t, 1 H),7.61 (d, 1 H), 7.39-7.19 (m, 3 H), 6.18 (s,1 H), 4.02 (s, 2 H), 3.39 (d, 2 H), 2.92 (s,3 H), 2.17-1.98 (m, 4 H), 1.99-1.75 (m,3 H), 1.61-1.42 (m, 2 H);HPLC-MS: 1.92 min, 436 [M + H]+ |
| 2-14 | 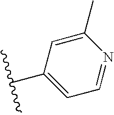 | H | 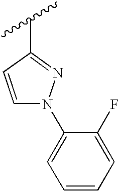 | ¹H NMR (400 MHz, DMSO-d6): δ 8.67 (d,1 H), 8.10-8.07 (m, 1 H), 7.90-7.86 (m,2 H), 7.76 (dt, 1 H), 7.40-7.18 (m, 1 H),5.86 (d, 1 H), 4.06 (s, 2 H), 3.05 (d, 2 H),2.69 (s, 3 H), 2.07-1.58 (m, 8 H), 1.24-1.08(m, 1 H);UPLC-MS: 0.63 min, 436 [M + H]+ |
| 2-15 | 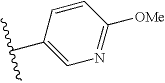 | H | 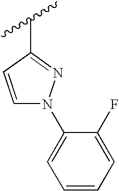 | ¹H NMR (400 MHz, DMSO-d6): δ 8.28 (d,1 H), 8.03 (dd, 1 H), 7.89 (t, 1 H), 7.77 (dt,1 H), 7.40-7.17 (m, 3 H), 6.86 (d, 1 H), 5.86(d, 1 H), 3.90 (s, 2 H), 3.84 (s, 3 H), 3.03(d, 2 H), 2.02-1.58 (m, 8 H), 1.24-1.13 (m,1 H);UPLC-MS: 0.81 min, 452 [M + H]+. |
| 2-16 | 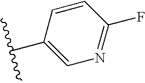 | H | 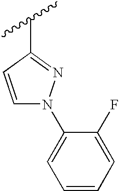 | ¹H NMR (400 MHz, DMSO-d6): δ 8.38-8.34(m, 1 H), 8.31-8.24 (m, 1 H), 7.90-7.86(t, 1 H), 7.88 (t, 1 H), 7.76 (t, 1 H),7.40-7.31 (m, 1 H), 7.30-7.16 (m, 3 H),5.84 (s, 1 H), 3.94 (s, 2 H), 3.02 (d, 2 H),1.97 (d, 2 H), 1.86 (d, 2 H), 1.72-1.58 (m,3 H), 1.24-1.11 (m, 2 H);UPLC-MS: 0.79 min, 440 [M + H]+ |
| 2-17 | 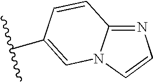 | H | 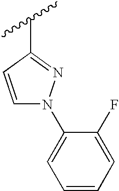 | ¹H NMR (400 MHz, DMSO-d6): δ 9.21 (brs, 1 H), 8.39-8.34 (m, 2 H), 8.21 (d, 1 H),8.02 (d, 1 H), 7.89 (t, 1 H), 7.78 (dt, 1 H),7.41-7.19 (m, 3 H), 5.89 (d, 1 H), 4.00 (s,2 H), 3.06 (d, 2 H), 2.07-1.70 (m, 7 H),1.25-1.10 (m, 2 H);UPLC-MS: 0.63 min, 461 [M + H]+ |
| 2-18 | 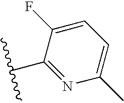 | H | 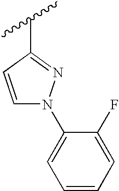 | ¹H NMR (400 MHz, DMSO-d6): δ 7.88 (t3 H), 7.79-7.68 (m, 2 H), 7.40-7.16 (m,4 H), 5.84 (d, 1 H), 4.28 (br s, 1 H), 3.97 (s,2 H), 3.02 (d, 2 H), 2.44 (s, 3 H), 2.07-1.62(m, 7 H), 1.30-1.16 (m, 2 H);UPLC-MS: 0.91 min, 461 [M + H]+ |
| 2-19 | 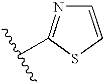 | H | 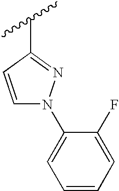 | ¹H NMR (400 MHz, DMSO-d6): δ 7.89 (t1 H), 7.78 (dt, 1 H), 7.48 (d, 1 H), 7.33 (d,1 H), 7.39-7.19 (m, 3 H), 5.86 (d, 1 H), 4.30(br s, 1 H), 4.06 (s, 2 H), 3.05 (d, 2 H),2.07-1.61 (m, 7 H), 1.29-1.15 (m, 2 H);UPLC-MS: 0.80 min, 428 [M + H]+ |
| 2-20 | 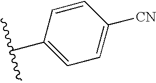 | H | 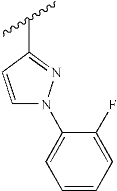 | ¹H NMR (400 MHz, DMSO-d6): δ 7.90-7.74(m, 5 H), 7.39-7.13 (m, 3 H), 5.84 (d,1 H), 4.17 (brs, 1 H), 3.97 (s, 2 H), 3.05 (d,2 H), 2.02-1.58 (m, 7 H), 1.18-1.14 (m,2 H);UPLC-MS: 0.83 min, 446 [M + H]+ |
| 2-21 | 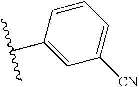 | H | 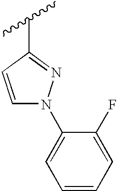 | ¹H NMR (400 MHz, DMSO-d6): δ 8.04(1 H, dt), 7.97-8.00 (1 H, m), 7.89 (1 H, t),7.78 (1 H, td), 7.56-7.64 (2 H, m), 7.36(1 H, ddd), 7.28 (1 H, td), 7.17-7.23 (1 H,m), 5.84 (1 H, d), 3.96 (2 H, s), 3.04 (2 H,d), 1.83-2.02 (4 H, m), 1.60-1.74 (3 H, m),1.13-1.28 (2 H, m);UPLC-MS: 0.83 min, 446 [M + H]+ |
| 2-22 | 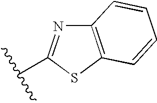 | H | 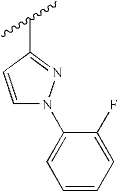 | ¹H NMR (400 MHz, DMSO-d6): δ 8.00(dq, 1 H), 7.90 (t, 1 H), 7.81-7.76 (m, 1 H),7.48-7.42 (m, 2 H), 7.40-7.18 (m, 4 H),5.87 (d, 1 H), 4.60 (br s, 1 H), 3.07 (d, 2 H),2.13-1.63 (m, 7 H), 1.35-1.22 (m, 2 H);UPLC-MS: 0.90 min, 478 [M + H]+ |
| 2-23 | 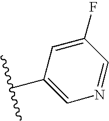 | H | 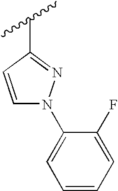 | ¹H NMR (400 MHz, DMSO-d6): δ 8.71 (brs, 1 H), 8.36 (d, 1 H), 8.02 (dt, 1 H), 7.90 (t,1 H), 7.78 (dt, 1 H), 7.40-7.20 (m, 3 H),5.86 (d, 1 H), 5.16 (br s, 1 H), 3.99 (s, 2 H),3.06 (d, 2 H), 2.05-1.61 (m, 7 H), 1.31-1.15(m, 2 H);UPLC-MS: 0.79 min, 440 [M + H]+ |
| 2-24 | 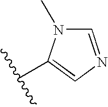 | H | 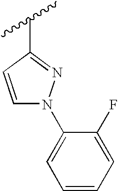 | ¹H NMR (400 MHz, DMSO-d6): δ 9.06 (s,1 H), 7.89 (t, 2 H), 7.84 (d, 1 H), 7.40-7.18(m, 3 H), 5.86 (d, 1 H), 3.97 (br s, 1 H),3.87 (s, 2 H), 3.76 (s, 3 H), 3.03 (d, 1 H),2.12-1.62 (m, 7 H), 1.18-1.03 (m, 2 H);UPLC-MS: 0.61 min, 425 [M + H]+ |
| 2-25 | 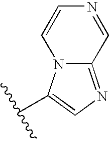 | H | 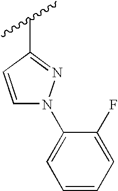 | ¹H NMR (400 MHz, DMSO-d6): δ 9.16 (d,1 H), 8.49 (dd, 1 H), 8.01 (d, 1 H), 8.00 (s,1 H), 7.90 (t, 1 H), 7.76 (dt, 1 H), 7.40-7.19(m, 3 H), 5.86 (d, 1 H), 4.78 (br s, 1 H),3.96 (s, 2 H), 3.04 (d, 2 H), 2.24-1.63 (m,7 H), 1.25-1.10 9 m, 2 H);UPLC-MS: 0.68 min, 462 [M + H]+ |
| 2-26 | 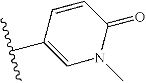 | H | 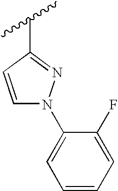 | ¹H NMR (400 MHz, DMSO-d6): δ 7.90-7.74(m, 4 H), 7.40-7.18 (m, 3 H), 6.43 (d,1 H), 5.85 (d, 1 H), 4.30 (br s, 1 H), 3.76 (s,2 H), 3.43 (s, 3 H), 3.03 (d, 1 H), 2.01-1.56(m, 7 H), 1.20-1.06 (m, 2 H). |
| 2-27 | 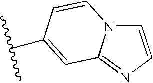 | H | 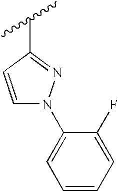 | ¹H NMR (400 MHz, DMSO-d6): δ 8.89 (d,1 H), 8.22 (br s, 1 H), 8.10 (d, 1 H), 8.02 (brs, 1 H), 7.92-7.86 (m, 2 H), 7.78 (dt, 1 H),7.43-7.18 (m, 3 H), 5.86 (d, 1 H), 4.08 (s,2 H), 3.93 (br s, 1 H), 3.07 (d, 2 H),2.08-1.59 (m, 7 H), 1.39-1.10 (m, 2 H);UPLC-MS: 0.63 min, 461 [M + H]+ |
| 2-28 | 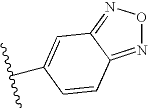 | H | 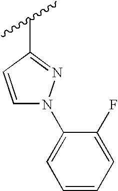 | ¹H NMR (400 MHz, DMSO-d6): δ 8.40(dd, 1 H), 8.11 (dd, 1 H), 7.89 (t, 1 H), 7.78(dt, 1 H), 7.70 (dd, 1 H), 7.39-7.18 (m, 3 H),5.85 (d, 1 H), 4.06 (s, 2 H), 4.00 (br s, 1 H),3.04 (d, 2 H), 2.06-1.61 (m, 7 H), 1.29-1.14(m, 2 H);UPLC-MS: 0.84 min, 463 [M + H]+ |
| 2-29 | 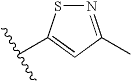 | H | 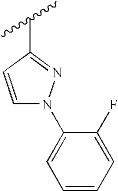 | ¹H NMR (400 MHz, DMSO-d6): δ 7.90 (t,1 H), 7.78 (dt, 1 H), 7.41-7.17 (m, 3 H),6.72 (s, 1 H), 5.86 (d, 1 H), 4.50 (br s, 1 H),3.96 (s, 2 H), 2.34 (d, 2 H), 2.06-1.65 (m,7 H), 1.26-1.12 (m, 2 H);UPLC-MS: 0.79 min, 442 [M + H]+ |
| 2-30 | 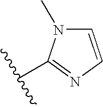 | H | 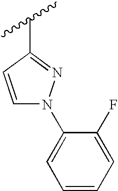 | ¹H NMR (400 MHz, DMSO-d6): δ 7.89 (t,1 H), 7.77 (dt, 1 H), 7.54 (d, 1 H), 7.46 (brs, 1 H), 7.40-7.33 (m, 1 H), 7.30-7.17 (m,2 H), 5.85 (d, 1 H), 4.04 (s, 2 H), 3.71 (s,3 H), 3.03 (d, 2 H), 2.13-1.66 (m, 7 H),1.22-1.06 (m, 2 H);UPLC-MS: 0.65 min, 425 [M + H]+ |
| 2-31 | 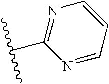 | H | 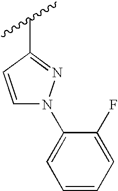 | ¹H NMR (400 MHz, DMSO-d6): δ 8.72 (d,2 H), 7.89 (t, 1 H), 7.78 (dt, 1 H), 7.40-7.16(m, 4 H), 5.86 (d, 1 H), 4.02 (s, 2 H), 4.00(br s, 1 H), 3.06 (d, 2 H), 2.03-1.60 (m,7 H), 1.32-1.14 (m, 2 H);UPLC-MS: 0.71 min, 422 [M + H]+ |
| 2-32 | 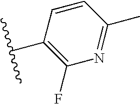 | H | 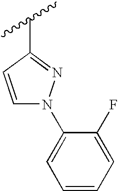 | ¹H NMR (400 MHz, DMSO-d6): δ 8.01(dd, 1 H), 7.79 (t, 1 H), 7.77 (dt, 1 H), 7.36(dd, 1 H), 7.30-7.17 (m, 3 H), 5.84 (d, 1 H),4.16 (br s, 1 H), 3.86 (s, 2 H), 3.02 (t, 2 H),2.26 (s, 3 H), 2.06-1.61 (m, 7 H), 1.20-1.10(m, 2 H);UPLC-MS: 0.79 min, 454 [M + H]+ |
| 2-33 | 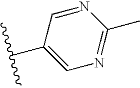 | H | 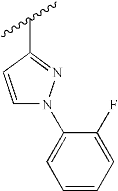 | ¹H NMR (400 MHz, DMSO-d6): δ 8.93 (s,2 H), 7.90 (t, 1 H), 7.78 (dt, 1 H), 7.40-7.18(m, 3 H), 5.86 (d, 1 H), 4.50 (br s, 1 H),3.97 (s, 2 H), 3.06 (t, 2 H), 2.60 (s, 3 H),2.04-1.61 (m, 7 H), 1.25-1.13 (m, 2 H);UPLC-MS: 0.72 min, 437 [M + H]+ |
| 2-34 | 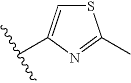 | H | 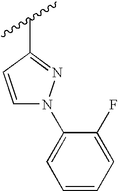 | ¹H NMR (400 MHz, DMSO-d6): δ 7.89 (t,1 H), 7.78 (dd, 1 H), 7.24 (s, 1 H), 7.40-7.18(m, 3 H), 5.85 (d, 1 H), 3.99 (s, 2 H),3.83 (br s, 1 H), 3.06 (d, 2 H), 2.64 (s, 3 H),2.05-1.61 (m, 7 H), 1.28-1.15 (m, 2 H);UPLC-MS: 0.82 min, 442 [M + H]+ |
| 2-35 | 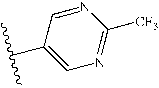 | H | 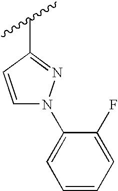 | ¹H NMR (400 MHz, DMSO-d6): δ 9.25 (s,2 H), 7.90 (br s, 1 H), 7.78 (t, 1 H), 7.41-7.16(m, 3 H), 5.85 (br s, 1 H), 4.16 (br s,1 H), 4.06 (s, 2 H), 3.04 (m, 2 H), 2.09-1.61(m, 7 H), 1.27-1.10 (m, 2 H);UPLC-MS: 0.83 min, 491 [M + H]+ |
| 2-36 | 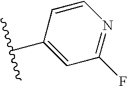 | H | 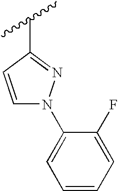 | ¹H NMR (400 MHz, DMSO-d6): δ 8.16 (d,1 H), 7.88 (t, 1 H), 7.76 (t, 1 H), 7.62 (d,1 H), 7.35 (dd, 1 H), 7.30-7.16 (m, 3 H),5.84 (d, 1 H), 3.94 (s, 2 H), 3.02 (d, 1 H),2.02-1.58 (m, 7 H), 1.24-1.13 (m, 2 H);UPLC-MS: 0.79 min, 440 [M + H]+ |
| 2-37 | 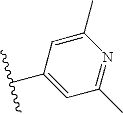 | H | 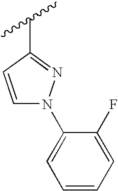 | ¹H NMR (400 MHz, DMSO-d6): δ 7.93-7.74(m, 4 H), 7.43-7.18 (m, 3 H), 5.85 (brs, 1 H), 4.04 (s, 2 H), 3.04 (d, 2 H), 2.66 (s,6 H), 2.07-1.63 (m, 7 H), 1.24-1.07 (m,2 H);UPLC-MS: 0.63 min, 450 [M + H]+ |
| 2-38 | 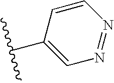 | H | 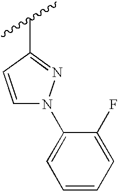 | ¹H NMR (400 MHz, DMSO-d6): δ 9.57(dd, 1 H), 9.24 (dd, 1 H), 8.00 (dd, 1 H),7.89 (t, 1 H), 7.78 (dt, 1 H), 7.37 (ddd, 1 H),7.32-7.18 (m, 2 H), 5.86 (d, 1 H), 4.05 (s,2 H), 3.06 (d, 2 H), 2.06-1.59 (m, 7 H),1.28-1.13 (m, 2 H);UPLC-MS: 0.68 min, 423 [M + H]+ |
| 2-39 | 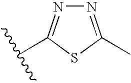 | H | 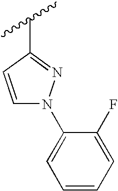 | ¹H NMR (400 MHz, DMSO-d6): δ 7.89 (t,1 H), 7.78 (dt, 1 H), 7.39-7.17 (m, 3 H),5.85 (d, 1 H), 4.06 (s, 2 H), 3.98 (br s, 1 H),3.06 (d, 2 H), 2.64 (s, 2 H), 2.08-1.57 (m,7 H), 1.30-1.17 (m, 3 H);UPLC-MS: 0.75 min, 443 [M + H]+ |
| 2-40 | 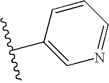 | H | 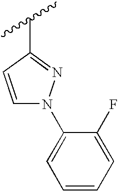 | ¹H NMR (400 MHz, DMSO-d6): δ 9.04(1 H, d), 8.53-8.63 (2 H, m), 7.94 (1 H, dd),7.91 (1 H, t), 7.78 (1 H, td), 7.38 (1 H, ddd),7.29 (1 H, td), 7.18-7.26 (1 H, m), 5.88(1 H, d), 4.04 (2 H, s), 3.06 (2 H, d), 1.85-2.05(4 H, m), 1.62-1.77 (3 H, m),1.13-1.27 (2 H, m);UPLC-MS: 0.69 min, 422 [M + H]+ and211 [M + 2 H]2+ |
| 2-41 | 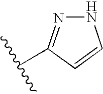 | H | 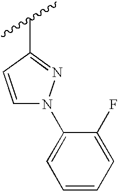 | 1H NMR (400 MHz, DMSO-d6): δ 7.90(1 H, t), 7.78 (1 H, td), 7.69 (1 H, d), 7.37(1 H, ddd), 7.25-7.32 (1 H, m), 7.16-7.25(1 H, m), 6.44 (1 H, d), 5.87 (1 H, d), 3.84(2 H, s), 3.06 (2 H, d), 1.81-2.00 (4 H, m),1.59-1.74 (3 H, m), 1.13-1.28 (2 H, m);HPLC-MS: 2.34 min, 411 [M + H]+ |
| 2-42 | 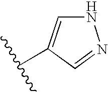 | H | 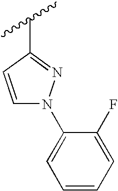 | 1H NMR (400 MHz, DMSO-d6): δ 7.90(1 H, t), 7.78 (2 H, td), 7.71 (2 H, s), 7.38(1 H, ddd), 7.28 (1 H, td), 7.17-7.25 (1 H,m), 5.86 (1 H, d), 3.75 (2 H, s), 3.04 (2 H,d), 1.82-2.00 (4 H, m), 1.59-1.73 (3 H, m),1.10-1.24 (2 H, m);HPLC-MS: 2.32 min, 411 [M + H]+, 206[M + 2 H]2+ |
| 2-43 | 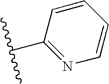 | H | 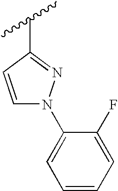 | 1H NMR (400 MHz, DMSO-d6): δ 1.13-1.26(m, 2 H) 1.58-1.70 (m, 3 H) 1.78-1.88(m, 2 H) 1.91-1.99 (m, 2 H) 3.03-3.07(d, 2 H) 3.96-4.00 (m, 2 H) 5.87-5.90(d, 1 H) 7.09-7.14 (m, 1 H) 7.16 -7.23 (m,1 H) 7.22-7.28 (dt, 1 H) 7.30- 7.37 (m,1 H) 7.71-7.78 (dt, 1 H) 7.78-7.84 (dt, 1 H)7.87-7.91 (m, 1 H) 8.04-8.09 (m, 1 H)8.31-8.35 (m, 1 H);UPLC-MS: 0.84 min, 422 [M + H]+ |
| 2-44 | 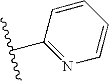 | H | 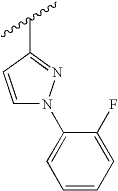 | 1H NMR (500 MHz, CDCl₃): δ 2.02 (d,8 H) 3.50 (d, 2 H) 4.00-4.03 (m, 2 H) 4.12(brs, 1 H) 5.84 (d, 1 H) 7.03-7.06 (m, 1 H)7.11-7.25 (m, 3 H) 7.68-7.75 (m, 1 H)7.84-7.91 (m, 2 H) 8.24 (d, 1 H) 8.33 (d,1 H);UPLC-MS: 0.97 min, 440 [M + H]+ |
| 2-45 | 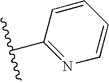 | H | 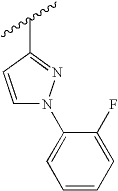 | 1H NMR (500 MHz, DMSO-d6): δ 1.72-2.03(m, 7 H) 2.61-2.64 (m, 1 H) 3.42 (d,2 H) 3.95 (br. s., 1 H) 4.05 (s, 2 H) 5.91(d, 1 H) 7.14 (dd, 1 H) 7.17-7.23 (m, 1 H)7.26 (t, 1 H) 7.35 (dd, 1 H) 7.75-7.86 (m,2 H) 7.89 (t, 1 H) 8.09 (d, 1 H) 8.35 (d,1 H). |
| 2-46 | 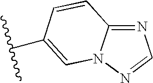 | H | 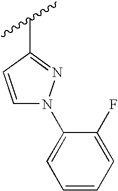 | 1H NMR (400 MHz, DMSO-d6): δ 9.10(1 H, dd), 8.51 (1 H, s), 8.21 (1 H, dd), 7.94(1 H, td), 7.58 (1 H, d), 7.91-7.89 (1 H,m), 7.82-7.76 (1 H, td), 7.41-7.34 (1 H,ddd), 7.32-7.26 (1 H, td), 7.25-7.18 (1 H,m), 5.86 (1 H, d), 4.02 (1 H, s), 3.06 (2 H,d), 2.02 (2 H, d), 1.92 (2 H, d), 1.77-1.61(3 H, m), 1.29-1.15 (2 H, m);UPLC-MS: 0.80 min, 462 [M + H]⁺ |
| 2-47 | 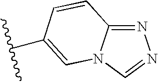 | H | 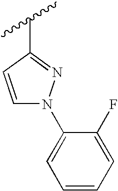 | 1H NMR (400 MHz, DMSO-d6): δ 9.35(1 H, s), 8.91-8.86 (1 H, m), 8.01-7.89 (3 H,m), 7.78 (1 H, td), 7.40-7.34 (1 H, ddd),7.29 (1 H, td), 7.25-7.19 (1 H, m), 5.86(1 H, d), 3.98 (1 H, s), 3.06 (2 H, d), 2.03(2 H, d), 1.92 (2 H, d), 1.79-1.64 (3 H, m),1.28-1.13 (2 H, m);UPLC-MS: 0.84 min, 462 [M + H]⁺ |
| 2-48 | 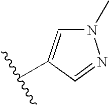 | H | 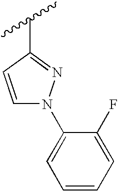 | 1H NMR (400 MHz, DMSO-d6): δ 7.92-7.88(1 H, m), 7.82 (1 H, d), 7.81-7.75(1 H, m), 7.51-7.50 (1 H, m), 7.41-7.34 (1 H,m), 7.25-7.18 (1 H, m), 5.86 (1 H, d), 3.82(3 H, s), 3.74 (2 H, s), 3.04 (2 H, d), 1.97-1.83(4 H, m), 1.72-1.62 (3 H, m),1.23-1.09 (2 H, m);UPLC-MS: 0.84 min, 425 [M + H]⁺ |
| 2-49 | 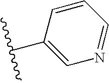 | H | 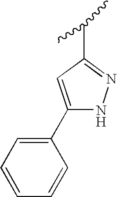 | ¹H NMR (400 MHz, DMSO-d6): δ 8.88 (d,1 H), 8.44 (dd, 1 H), 8.23 (m, 1 H), 7.83 (m,2 H), 7.65 (m, 1 H), 7.55-7.48 (m, 2 H),6.24 (s, 1 H), 4.00 (s, 2 H), 3.09 (m, 2 H),2.04-1.63 (m, 7 H), 1.24 (m, 2 H);UPLC-MS: 0.57 min, 404 [M + H]+. |
| 2-50 | 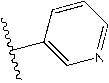 | H | 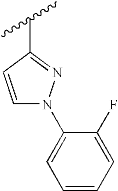 | ¹H NMR (400 MHz, DMSO-d6): δ 8.97 (d,1 H), 8.56-8.51 (m, 1 H), 8.41-8.34 (m,1 H), 7.90 (t, 1 H), 7.86-7.75 (m, 2 H), 7.40-7.18(m, 3 H), 5.87 (d, 1 H), 4.30 (br s,1 H), 3.90 (s, 2 H), 3.06 (d, 2 H), 2.10-2.00(m, 2 H), 1.83-1.60 (m, 5 H), 1.34-1.21 (m,2 H);UPLC-MS: 0.68 min, 422 [M + H]+ |
| 2-51 | 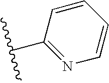 | H | 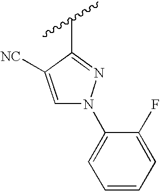 | 1H NMR (400 MHz, CDCl₃): δ 8.33-8.37(m, 1 H), 8.27-8.31 (m, 1 H), 8.19 (d,1 H), 7.86-7.93 (m, 1 H), 7.73 (ddd, 7.14,2.02 Hz, 1 H), 7.20-7.33 (m, 3 H), 7.00-7.09(m, 1 H), 4.24 (t, 1 H), 4.08 (s, 2 H),3.32 (t, 2 H), 1.99-2.09 (m, 4 H), 1.74-1.95(m, 3 H), 1.19-1.33 (m, 2 H); HPLC-MS: 2.38 min, 447.2 [M + H]+ |
| 2-52 | 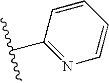 | H | 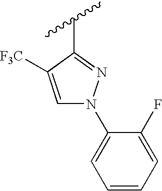 | 1H NMR (400 MHz, CDCl3): δ 1.18-1.36(m, 2 H), 1.76-1.95 (m, 3 H), 1.98-2.09(m, 4 H), 3.25-3.33 (m, 2 H), 4.01 (t, 1 H),4.08 (s, 2 H), 7.06 (ddd, 1 H), 7.19-7.31(m, 3 H), 7.73 (ddd, 1 H), 7.87-7.93 (m,1 H), 8.09-8.11 (m, 1 H), 8.30 (dt, 1 H),8.34-8.37 (m, 1 H);HPLC-MS: 2.85 min, 490.1 [M + H]+ |
| 2-53 | 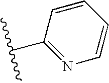 | H | 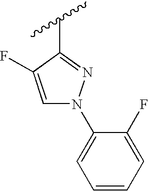 | 1H NMR (400 MHz, CDCl3): δ 8.35 (dt,1 H), 8.29 (d, 1 H), 7.79-7.90 (m, 2 H), 7.73(ddd, 1 H), 7.10-7.26 (m, 3 H), 7.01-7.08(m, 1 H), 3.73 (brs, 1 H), 4.08 (s, 2 H),3.28 (d, 2 H), 1.98-2.11 (m, 4 H), 1.72-1.95(m, 3 H), 1.19-1.35 (m, 2 H);UPLC-MS: 0.84 min, 220.57 [M + 2 H]2+,440.07 [M + H]+ |
| 2-54 | 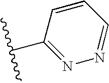 | H | 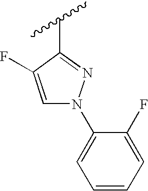 | ¹H-NMR (400 MHz, CDCl3): δ 8.96 (dd,1 H), 8.57 (dd, 1 H), 7.77-7.90 (m, 2 H),7.50 (dd, 1 H), 7.07-7.26 (m, 3 H), 4.23(s, 2 H), 3.75 (d, 1 H), 3.28 (t, 2 H), 2.00-2.12(m, 4 H), 1.75-1.97 (m, 3 H), 1.18-1.34(m, 2 H); R_{f} = 0.2 (AcOEt5/cyclohexane 5);UPLC-MS: 0.76 min, 441.01 [M + H]⁺ |
| 2-55 | 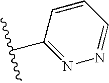 | H | 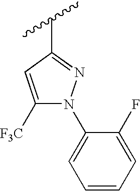 | 1H NMR (400 MHz, CDCl3): δ 1.12-1.25(m, 2 H), 1.67-1.82 (m, 1 H), 1.83-2.06(m, 6 H), 3.07 (t, 2 H,) 3.76 (t, 1 H), 4.20(s, 2 H), 5.78 (s, 1 H), 7.28-7.38 (m, 2 H),7.46-7.59 (m, 3 H), 8.57 (dd, 1 H,) 8.97(dd, 1 H);UPLC-MS: 0.74 min, 491 [M + H]+ |

### Example 3

### Preparation of Compounds of Formula (IIc)

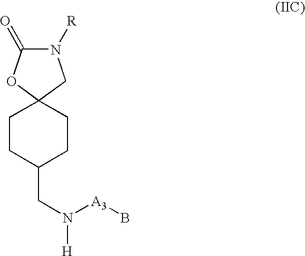

### Example 3-1

### (Trans)-8-{[(3-phenyl-5-isoxazolyl)amino]methyl}-3-(2-pyridinyl)-1-oxa-3-azaspiro[4.5]decan-2-one hydrochloride

(Trans)-2-oxo-3-(2-pyridinyl)-1-oxa-3-azaspiro[4.5]decane-8-carbaldehyde (this may be prepared as described for intermediate 26, 68 mg, 0.261 mmol), 3-phenyl-5-isoxazolamine (54.4 mg, 0.340 mmol), and chlorotitanium triisopropoxide (0.168 ml, 0.705 mmol) were mixed in dichloromethane (0.75 ml) at room temperature under nitrogen atmosphere for 14 hours. Thereafter sodium triacetoxyborohydride (277 mg, 1.306 mmol) and acetic acid (0.075 ml, 1.310 mmol) were added and the mixture stirred for 14 hours. The mixture was diluted with dichloromethane (20 ml) and saturated aqueous sodium hydrogencarbonate (7 ml) was added. The mixture was stirred for 30 minutes and then filtered. The filtrate was washed with saturated aqueous sodium hydrogencarbonate (8 ml) and brine (8 ml). The organic phase was passed through a hydrophobic PTFE frit and evaporated. The crude was columned from silica using cyclohexane/ethyl acetate 9/1 to pure ethyl acetate. The desired product was obtained as a mixture with an unidentified by-product. Thus, this mixture was re-submitted to column chromatography on silica using cyclohexane/ethyl acetate: 4/1 to 2/1. 12 mg of the desired product were collected. Further purification by means of an SCX resin eluting with dichloromethane, methanol and 2M ammonia in methanol was undertaken. Evaporation of the basic fractions yielded 10.7 mg of (trans)-8-{[(3-phenyl-5-isoxazolyl)amino]methyl}-3-(2-pyridinyl)-1-oxa-3-azaspiro[4.5]decan-2-one.

1H-NMR (400 MHz, CDCl₃): ° 8.35 (1H, ddd), 8.29 (1H, dt), 7.74-7.79 (2H, m), 7.73 (1H, ddd), 7.40-7.49 (3H, m), 7.06 (1H, ddd), 5.30 (1H, s), 4.63 (1H, t), 4.07 (2H, s), 3.18 (2H, t), 1.96-2.10 (4H, m), 1.89 (2H, td), 1.71-1.82 (1H, m), 1.17-1.33 (2H, m); UPLC-MS: 0.78 min, 405 [M+H]+.

This was dissolved in dichloromethane and was treated with 1M hydrogenchloride in diethyl ether, followed by evaporation of the solvent and drying under vacuum to give the title compound (11.8 mg).

### Example 3-2

### (Trans)-3-phenyl-8-{[(3-phenyl-5-isoxazolyl)amino]methyl}-1-oxa-3-azaspiro[4.5]decan-2-one

(Trans)-2-oxo-3-phenyl-1-oxa-3-azaspiro[4.5]decane-8-carbaldehyde (prepared in a similar fashion to Intermediate 7, 35 mg, 0.135 mmol) and 3-phenyl-5-isoxazolamine (21.62 mg, 0.135 mmol) were dissolved in dry tetrahydrofuran (1.5 ml). Titanium(IV) isopropoxide (0.079 ml, 0.270 mmol) was added and the mixture was stirred at room temperature overnight. Sodium borohydride (15.32 mg, 0.405 mmol) and EtOH (0.1 mL) were added and the mixture was stirred at r.t. for 7 hours. 1 drop of water was added and the mixture was concentrated under reduced pressure to give a residue that was partitioned between H2O/DCM. DCM extracts (3×1 mL) were combined and concentrated under reduced pressure to give a residue that was purified by silica gel chromatography (Biotage SP1, 12+M) eluting with DCM:MeOH 100:0 to 95:5. Product fractions were combined and concentrated under reduced pressure to give 13 mg of a white solid that was further purified by MDAP. Product fractions were filtered through an SCX cartridge (1 g) eluting with MeOH then 2M ammonia in MeOH. MeOH fractions were concentrated under reduced pressure to afford the title compound as a white solid (3.7 mg, 7%); All the analytical data are set forth in the following Table 3-1 and in which R, A₃ and B are:

| | |
|---|---|
| | (IIC) |
| 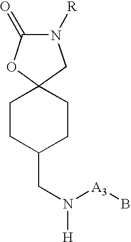 | |

| Cpd. | | | |
|---|---|---|---|
| No. | R | A3—B | Analytical Data |
| 3-1 | 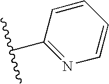 | 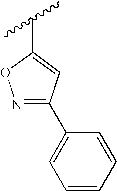 | 1H-NMR (400 MHz, DMSO-d6): δ 8.37(1 H, ddd), 8.10 (1 H, d), 7.84 (1 H, ddd),7.71-7.78 (2 H, m), 7.41-7.50 (3 H, m),7.15 (1 H, ddd), 5.57 (1 H, s), 4.01 (2 H, s),3.01-3.13 (2 H, m), 1.92-2.03 (2 H, m),1.77-1.87 (2 H, m), 1.59-1.74 (3 H, m),1.19-1.32 (2 H, m), 0.82-0.91 (2 H, m). |
| 3-2 | 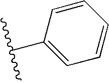 | 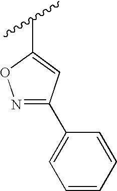 | 1H-NMR (400 MHz, CDCl₃): δ 1.13-1.32(m, 2 H) 1.70-1.96 (m, 3 H) 2.02 (m, 4 H)3.21 (t, 2 H) 3.82 (s, 2 H) 4.61-4.68 (m,1 H) 5.31 (s, 1 H) 7.12-7.19 (m, 1 H) 7.35-7.48(m, 5 H) 7.55-7.62 (m, 2 H)7.72-7.79 (m, 2 H)UPLC-MS 0.80 min, 404 [M + H]+ |

### Example 4

### In Vitro Profile

The in vitro assessment of the NPY Y5 antagonist compounds used different assay systems to determine the potency and affinities against the NPY Y5 receptor.

The affinities of the compounds of the invention for the NPY Y5 receptor may be determined by the binding assays described below. Such affinity is typically calculated from the IC₅₀ obtained in competition experiments as the concentration of a compound necessary to displace 50% of the radiolabeled ligand from the receptor, and is reported as a “Kᵢ” value calculated by the following equation:

$${K}_{i} = \frac{{IC}_{50}}{1 + L / {K}_{D}}$$

where L=radioligand and K_{D}=affinity of radioligand for receptor (Cheng and Prusoff, Biochem. Pharmacol. 22: 3099, 1973). In the context of the present invention pKi values (corresponding to the antilogarithm of Ki) are used instead of Ki; pKi results are only estimated to be accurate to about 0.3-0.5.

The functional activity of the compounds of the invention for the NPY Y5 receptor may be determined by the FLIPR/Ca²⁺ assay as described below. Such potency is typically calculated from the IC₅₀ obtained in FLIPR experiments as the concentration of a compound necessary to decrease 50% of the calcium release following cells exposure to a concentration of PYY eliciting 80% response (i.e. EC80), and is reported as a “fKᵢ” value calculated by the following equation:

$${fK}_{i} = \frac{{IC}_{50}}{1 + EC 80 / EC 50}$$

where EC80 and EC50 corresponding to the agonist (PYY) concentrations that eliciting 80% and 50% response, respectively (corresponding to the Cheng and Prusoff equation). In the context of the present invention pfKi values (corresponding to the antilogarithm of fKi) are used instead of fKi; pfKi results are only estimated to be accurate to about 0.3-0.5.

### Functional Activity at Recombinant Human NPY Y5 Receptor

The functional activity at the human NPY Y5 receptor stably expressed in HEK293 cells was assessed using FLIPR/Ca²⁺ methodology (cell line name: HEK 293 signal-hNPY Y5/G16z49). The assay is configured to re-direct receptor-mediated signalling to the calcium release from intracellular stores via the promiscuous Gα16z49 protein.

PYY (peptide YY) is an endogenous agonist and can activate the receptor, thereupon causing an increase in the level of calcium in the cells sensed by Fluo4-AM and measured by FLIPR. Antagonist effects are monitored by the blockade or decrease in calcium release once cells co-expressing hNPY Y5 receptor and Gα16z49 are exposed to a concentration of PYY eliciting 80% response (i.e. EC80). A non-linear, 4 parameter logistic curve-fit of the data generated pIC₅₀ value. Applying the Cheng-Prusoff equation to antagonist concentration-response for inhibition of fixed PYY concentration yielded the fpKi values.

Cells are cultured in DMEM/F12 supplemented with 10% FBS, 2 mM Glutamine, 200 μg/mL hygromycin B and 500 μg/mL G418. The day before a FLIPR experiment, cells are plated out into 384-well Poly-D-Lysine coated FLIPR plates at a density of 200,000 cells/mL corrects to give 10,000 cells per 50 μL per well using medium without antibiotics.

On the day of experiment, cells are washed with an assay buffer containing 20 mM HEPES/NaOH, 145 mM NaCl, 5 mM KCl, 1 mM MgCl₂, 2 mM CaCl₂, 1 g/L D-glucose and 2.5 mM probenecid, pH 7.3 and loaded with 2 μM Fluo-4 AM for 60 min at 37° C. and 5% CO₂. The excess of dye solution is removed by washing cells with buffer. Compound solutions, prepared by serially diluting compounds in neat DMSO and then a final 1:50 dilution step in assay buffer added with 0.05% pluronic acid, are added and incubated with the loaded cells for 30 min at 37° C. and 5% CO₂.

Cells are then put in the FLIPR for the stimulus addition corresponding to a concentration of PYY eliciting 80% of the response. The response of cells to the agonist is fast and measured for 2 min after PYY addition.

### Binding Affinities at Human and Rat NPY Y5 Receptors

The assays used to measure compound affinity to human and rat NPY Y5 receptors were binding assays using Scintillation Proximity Assay (SPA) technology. The SPA involves the coupling of cell membrane fragments, via their glycosylated residues, to the wheat germ agglutinin (WGA) present on the surface of SPA beads. This coupling mechanism immobilises receptors in close proximity to the scintillant within the SPA beads and binding to the receptors of a radiolabelled ligand can thus be measured directly without the need to separate bound from free ligand.

Binding experiments are carried out in 384-well plates. The assay buffer contains 50 mM HEPES/NaOH pH 7.4, 1 mM MgCl2, 2.5 mM CaCl2 and 0.05% pluronic acid. Specific binding is defined as the portion of [125I]-porcinePYY that is displaceable by 1 μM human PYY. A non-linear, 4 parameter logistic curve-fit of the data generated pIC₅₀ and pKi values.

### 125I—PYY Binding on Human NPY Y5 BacMam Membranes

Competition experiments are carried out in 384-well white with clear bottom plates in a final volume of 50 μL. PVT-WGA beads and membranes (prepared from HEK293F G0 cells) are diluted in assay buffer to have 2.5 mg/mL and 50 μg/mL, respectively and precoupled at 4° C. for 60 min. [125I]-PYY is added to the membrane-beads mix to achieve a concentration of 20 pM. 50 μL of the SPA mix is added to each well containing 0.5 μL compound solution. Compound solutions are prepared by serially diluting compounds in neat DMSO. The incubation lasted 3 hours at room temperature under gentle shaking. Then plates are left overnight at room temperature to allow the beads to settle and bound radioactivity is measured using Trilux MicroBeta.

### 125I—PYY Binding on Rat NPY Y5 BacMam Membranes

Competition experiments are carried out in 384-well white plates in a final volume of 30 μL. WGA-Polystyrene LEADseeker imaging beads and membranes (prepared from HEK293F G0 cells), are diluted in assay buffer to have 2.5 mg/mL and 30 μg/mL, respectively and precoupled at 4° C. for 60 min. [125I]-PYY is added to the membrane-beads mix to achieve a concentration of 75 pM. 30 μL of the SPA mix is added to each well containing 0.3 μL compounds solution. Compound solutions are prepared by serially diluting compounds in neat DMSO. The incubation lasted 3 hours at room temperature under gentle shaking. Then plates are left overnight at room temperature and bound radioactivity is measured using ViewLux.

All the compounds of formula (I) are believed to bind the NPY Y5 receptor. Preferred compounds show pKi comprised between 6 and 10 and fpKi comprised between 6 and 11 towards NPY Y5 receptor.

1. A compound selected from a group consisting of:
(trans)-8-({[1-(2-fluorophenyl)-1H-pyrazol-3-yl]amino}methyl)-3-(2-fluoro-3-pyridinyl)-1-oxa-3-azaspiro[4.5]decan-2-one;
(trans)-8-({[1-(2-fluorophenyl)-1H-pyrazol-3-yl]amino}methyl)-3-(3-pyridazinyl)-1-oxa-3-azaspiro[4.5]decan-2-one; and
(trans)-8-({[1-(2-fluorophenyl)-1H-pyrazol-3-yl]amino}methyl)-3-(1-methyl-1H-pyrazol-3-yl)-1-oxa-3-azaspiro[4.5]decan-2-one, or a pharmaceutically acceptable salt or solvate thereof.

2. (Trans)-8-({[1-(2-fluorophenyl)-1H-pyrazol-3-yl]amino}methyl)-3-(2-fluoro-3-pyridinyl)-1-oxa-3-azaspiro[4.5]decan-2-one or a pharmaceutically acceptable salt or solvate thereof.

3. (Trans)-8-({[1-(2-fluorophenyl)-1H-pyrazol-3-yl]amino}methyl)-3-(3-pyridazinyl)-1-oxa-3-azaspiro[4.5]decan-2-one or a pharmaceutically acceptable salt or solvate thereof.

4. (Trans)-8-({[1-(2-fluorophenyl)-1H-pyrazol-3-yl]amino}methyl)-3-(1-methyl-1H-pyrazol-3-yl)-1-oxa-3-azaspiro[4.5]decan-2-one or a pharmaceutically acceptable salt or solvate thereof.

5. A method of treating a condition for which modulation of NPY Y5 receptors is beneficial, which comprises administering to a mammal in need thereof an effective amount of a compound as claimed in claim 1.

6. A method as claimed in claim 5, wherein the condition is eating disorders.

7. A method as claimed in claim 6, wherein the condition is binge eating.

8. A method as claimed in claim 6, wherein the condition is obesity.

9. A method as claimed in claim 5, wherein the condition is depression.

10. A pharmaceutical composition comprising a compound as claimed in claim 1 and a pharmaceutically acceptable carrier.
